(19) United States
(12) Patent Application Publication
(10) Pub. No.: US 2025/0271393 A1
(43) Pub. Date: Aug. 28, 2025

(54) PORTABLE BIOSENSOR SYSTEM WITH VERTICAL GRAPHENE ARRAY

(71) Applicant: Lyten, Inc., San Jose, CA (US)

(72) Inventors: Eric Danielson, San Jose, CA (US); Bradley Napier, San Francisco, CA (US); Kevin Cheung, San Jose, CA (US); Daniel Jardine, Santa Clara, CA (US)

(21) Appl. No.: 19/203,794

(22) Filed: May 9, 2025

Related U.S. Application Data
(63) Continuation-in-part of application No. 17/182,045, filed on Feb. 22, 2021.
Application No. 17/182,045 is a continuation-in-part of application No. 16/887,293, filed on May 29, 2020, now Pat. No. 11,137,368.
Application No. 16/887,293 is a continuation-in-part of application No. 16/706,542, filed on Dec. 6, 2019, now Pat. No. 10,955,378.
Application No. 16/706,542 is a continuation of application No. 16/239,423, filed on Jan. 3, 2019, now Pat. No. 10,502,705.
(60) Provisional application No. 62/815,927, filed on Mar. 8, 2019.
(60) Provisional application No. 62/613,716, filed on Jan. 4, 2018.
(60) Provisional application No. 63/649,849, filed on May 20, 2024.
(60) Provisional application No. 62/979,095, filed on Feb. 20, 2020.
(60) Provisional application No. 63/088,541, filed on Oct. 7, 2020.

(51) Int. Cl. G01N 27/414; B01J 20/28; C01B 32/182; G01N 27/12; G01N 27/404; G01N 29/036; G01N 33/00

(52) U.S. Cl. CPC G01N 27/4141; B01J 20/28066; C01B 32/182; C01B 2204/04; C01B 2204/22; C01B 2204/32; G01N 27/127; G01N 27/4045; G01N 29/036; G01N 33/0037; G01N 33/0039; G01N 33/004; G01N 33/0044; G01N 2291/014

(57) ABSTRACT

The present disclosure provides an innovative biosensor system utilizing three-dimensional vertical graphene structures for highly sensitive analyte detection in field-deployable applications. The vertical graphene structures may be formed in-situ directly on the sensor substrate, potentially enabling on-site fabrication and customization. These structures, with their increased surface area and unique tree-like morphology, may offer improved binding sites for bioreceptors compared to conventional flat graphene sensors. This biosensor design may address limitations of existing biosensors by combining enhanced surface area, controlled sample handling, and advanced measurement techniques in a single, field-portable device. The potential for in-situ graphene formation may allow for rapid sensor deployment and adaptation. Additionally, the system's portability may enable on-site analysis in remote locations, potentially reducing the need for sample transport and laboratory-based testing.

### CROSS-REFERENCE TO RELATED APPLICATIONS

The present Application is a continuation-in-part application and claims priority to U.S. patent application Ser. No. 17/182,045 entitled “SENSING DEVICE FOR DETECTING ANALYTES IN PACKAGES,” filed on Feb. 22, 2021, which in turn is a continuation-in-part application and claims priority to U.S. patent application Ser. No. 16/887,293 entitled “RESONANT GAS SENSOR” filed on May 29, 2020, which claims priority to U.S. Provisional Patent Application No. 62/815,927 entitled “RESONANT GAS SENSOR” filed on Mar. 8, 2019 and is a continuation-in-part application of U.S. patent application Ser. No. 16/706,542 entitled “RESONANT GAS SENSOR” filed on Dec. 6, 2019, which is a continuation application of U.S. patent application Ser. No. 16/239,423 entitled “RESONANT GAS SENSOR” filed on Jan. 3, 2019, which claims priority to U.S. Provisional Patent Application No. 62/613,716 entitled “VOLATILES SENSOR” filed on Jan. 4, 2018, all of which are assigned to the assignee hereof. The disclosures of all prior Applications are considered part of and are incorporated by reference in this Patent Application in their respective entireties.

The present Application also claims priority to U.S. Provisional Patent Application No. 63/649,849 entitled “BIOMARKER DETECTION USING FUNCTIONALIZED BIOFETS,” filed on May 20, 2024, which is assigned to the assignee hereof. The disclosures of all prior Applications are considered part of and are incorporated by reference in this Patent Application in their respective entireties.

U.S. patent application Ser. No. 17/182,045 also claims priority to U.S. Provisional Patent Application No. 62/979,095 entitled “MULTIVARIATE IMPEDANCE SPECTROSCOPY SENSING” filed on Feb. 20, 2020, and to U.S. Provisional Patent Application No. 63/088,541 entitled “MULTIVARIATE CHEMICALLY-FUNCTIONALIZED CARBON-BASED RESONANT IMPEDANCE SPECTROSCOPY SENSOR ARRAYS” filed on Oct. 7, 2020, all of which are assigned to the assignee hereof. The disclosures of all prior Applications are considered part of and are incorporated by reference in this Patent Application in their respective entireties.

### FIELD OF THE INVENTION

The present disclosure relates to biosensor systems, and more particularly to a portable biosensor system utilizing a vertical graphene field effect transistor array for detecting analytes.

### BACKGROUND

Biosensors play a crucial role in detecting and quantifying biological analytes for various applications in healthcare, environmental monitoring, and research. However, current biosensing technologies often require complex laboratory equipment, specialized personnel, and time-consuming procedures, limiting their use in field or point-of-care settings. This presents a significant challenge in situations where rapid, on-site analysis is needed, such as disease diagnosis, environmental testing, or food safety monitoring.

Existing portable biosensor systems face several obstacles in achieving the sensitivity, specificity, and reliability of laboratory-based methods. These challenges include miniaturization of sensing components without compromising performance, integration of sample preparation and analysis steps, and development of user-friendly interfaces for non-expert operators. Additionally, many current systems struggle with issues related to sensor stability, cross-reactivity with interfering substances, and the ability to detect multiple analytes simultaneously.

For example, in emergency medical situations, current point-of-care biosensors may lack the sensitivity to detect low concentrations of critical biomarkers, potentially leading to missed diagnoses. Similarly, in environmental monitoring applications, existing portable systems often cannot match the detection limits of laboratory equipment, making it difficult to identify trace contaminants in water or soil samples. These limitations highlight the need for improved biosensor technologies that can bridge the gap between laboratory-grade performance and field-deployable functionality.

As such, there is thus a need for addressing these and/or other issues associated with the prior art.

### SUMMARY

In some aspects, the techniques described herein relate to a biosensor including: a substrate; an array of three-dimensional vertical graphene structures disposed on the substrate; electrodes in electrical contact with the vertical graphene structures; and a functionalization layer on surfaces of the vertical graphene structures, wherein the functionalization layer includes bioreceptors configured to bind to specific target analytes.

In some aspects, the techniques described herein relate to a biosensor, wherein the vertical graphene structures have a height ranging from 300 nm to 500 nm.

In some aspects, the techniques described herein relate to a biosensor, wherein the vertical graphene structures exhibit a jagged surface morphology.

In some aspects, the techniques described herein relate to a biosensor, wherein the substrate includes silicon dioxide.

In some aspects, the techniques described herein relate to a biosensor, wherein the electrodes include a source electrode and a drain electrode positioned on opposite sides of each vertical graphene structure.

In some aspects, the techniques described herein relate to a biosensor, further including a gate electrode positioned adjacent to the vertical graphene structures.

In some aspects, the techniques described herein relate to a biosensor, wherein: the gate electrode is configured to apply a voltage ranging from −0.1V to 0.9V; and a bias voltage of 50-300 mV is applied to the source and drain electrodes during measurement.

In some aspects, the techniques described herein relate to a biosensor, wherein the bioreceptors include at least one of antibodies, nucleic acids, or proteins.

In some aspects, the techniques described herein relate to a biosensor, further including a microfluidic system configured to deliver liquid samples to the functionalized vertical graphene structures.

In some aspects, the techniques described herein relate to a biosensor, wherein the microfluidic system includes: at least one sample well for containing a liquid sample; a microfluidic channel connecting the sample well to the functionalized vertical graphene structures; and a pump for controlling fluid flow through the microfluidic channel.

In some aspects, the techniques described herein relate to a biosensor, further including a flow sensor for monitoring fluid flow rates through the microfluidic system.

In some aspects, the techniques described herein relate to a biosensor, further including a measurement circuit configured to detect changes in electrical properties of the vertical graphene structures upon binding of target analytes to the bioreceptors.

In some aspects, the techniques described herein relate to a biosensor, wherein the measurement circuit is configured to perform current drift correction on sensor responses.

In some aspects, the techniques described herein relate to a biosensor, wherein the measurement circuit is configured to sweep through a range of applied voltages when measuring sensor responses.

In some aspects, the techniques described herein relate to a biosensor, further including a controller configured to: control exposure of liquid samples to the functionalized vertical graphene structures; measure sensor responses from the vertical graphene structures; and process data from the sensor responses to determine analyte concentrations in the liquid samples.

In some aspects, the techniques described herein relate to a biosensor, wherein the controller is further configured to certify minimal sensor performance before proceeding with sample exposure.

In some aspects, the techniques described herein relate to a biosensor, wherein the functionalization layer includes amine diazonium groups covalently bonded to the surfaces of the vertical graphene structures.

In some aspects, the techniques described herein relate to a biosensor, wherein the bioreceptors are attached to the amine diazonium groups via EDC/NHS (1-Ethyl-3-(3-dimethylaminopropyl) carbodiimide/N-hydroxysuccinimide) chemistry.

In some aspects, the techniques described herein relate to a biosensor, further including: a housing enclosing the substrate, vertical graphene structures, and electrodes; a display screen on an exterior surface of the housing for providing visual information to a user; and at least one microfluidic port on an exterior surface of the housing for introducing liquid samples.

In some aspects, the techniques described herein relate to a biosensor, further including a wireless communication module configured to transmit measurement data to a remote device.

In some aspects, the techniques described herein relate to a biosensor, wherein: the vertical graphene structures have a height ranging from 300 nm to 500 nm;

the vertical graphene structures exhibit a jagged surface morphology; and the vertical graphene structures are configured to extend into the liquid sample beyond the Debye screening length, enabling more efficient interaction with target analytes.

In some aspects, the techniques described herein relate to a biosensor, wherein: the vertical graphene structures are grown in-situ directly on the substrate; the in-situ growth process results in improved adhesion and electrical contact between the vertical graphene structures and the electrodes compared to monolayer graphene; and the vertical graphene structures have a non-uniform surface morphology with varying heights to increase surface area for analyte binding.

In some aspects, the techniques described herein relate to a biosensor, further including: a microfluidic system configured to deliver liquid samples to the functionalized vertical graphene structures; a measurement circuit configured to detect changes in electrical properties of the vertical graphene structures upon binding of target analytes to the bioreceptors; and a controller configured to process data from the sensor responses to determine analyte concentrations in the liquid samples.

In some aspects, the techniques described herein relate to a biosensor, wherein: the biosensor is configured as a field-deployable device; the field-deployable device includes a display for real-time data visualization, a PCB assembly for electronic control and signal processing, and microfluidic ports for sample introduction and management; and the field-deployable device is configured for on-site sample analysis without the need for complex laboratory equipment.

### BRIEF DESCRIPTION OF THE DRAWINGS

FIG. 1 illustrates a cross-sectional view of a sensor system, according to aspects of the present disclosure.

FIG. 2 illustrates a conventional flat graphene FET sensor system, according to an embodiment.

FIG. 3 illustrates a cross-section perspective view of a vertical graphene FET sensor system, according to aspects of the present disclosure.

FIG. 4 illustrates a top view of a sensor assembly, according to an embodiment.

FIG. 5 illustrates a flowchart of a method for fabricating vertical graphene, according to aspects of the present disclosure.

FIG. 6 illustrates a cross-sectional view of a sensor system with vertical graphene, according to an embodiment.

FIG. 7 illustrates a cross-sectional view of a sensor system with vertical graphene and platinum layer, according to aspects of the present disclosure.

FIG. 8 illustrates a top-down view of vertical graphene structures on different substrates, according to an embodiment.

FIG. 9 illustrates a top-down view of vertical graphene growth on different substrates, according to aspects of the present disclosure.

FIG. 10 illustrates a top-down view of vertical graphene growth on different substrates, according to an embodiment.

FIG. 11 illustrates a top-down view of a vertical graphene structure on a silicon dioxide substrate, according to aspects of the present disclosure.

FIG. 12 illustrates a flowchart of a measurement method for vertical graphene, according to an embodiment.

FIG. 13 illustrates an isometric view of a field deployable sampling unit, according to aspects of the present disclosure.

FIG. 14 illustrates an isometric view of a field deployable vertical graphene FET device, according to an embodiment.

FIG. 15-1 shows an example sensing device configured to detect analytes, according to some implementations.

FIG. 15-2 is an illustration depicting the sensing device of FIG. 15-1 coupled to a receptor, according to some implementations.

FIG. 15-3 is an illustration depicting the sensing device of FIG. 15-1 configured to detect analytes in a battery pack, according to some implementations.

FIG. 15-4 is an illustration depicting the sensing device of FIG. 15-1 configured to detect analytes in a battery pack, according to some implementations.

FIG. 15-5 is an illustration depicting reactions between one or more analytes and the sensing device of FIG. 15-1, according to some implementations.

FIG. 15-6 is a block diagram of an analyte detection system that includes the sensing device of FIG. 15-1, according to some implementations.

FIGS. 15-7A-15-7E show sensor arrays configured to detect analytes, according to various implementations.

FIG. 15-8 shows a flow chart depicting an example operation for fabricating at least some of the sensing devices disclosed herein, according to some implementations. FIG. 15-9 shows another sensor array, according to some implementations.

FIG. 15-10A shows an example sensor configuration, according to some implementations.

FIG. 15-10B shows an example sensor configuration, according to other implementations.

FIGS. 15-11A-15-11G show illustrations of various structured carbon materials that can be used in the sensing devices disclosed herein, according to some implementations.

FIGS. 15-12A-15-12F show example frequency responses of resonance impedance sensors to various analytes, according to some implementations.

FIG. 15-13A shows the real (Z′) impedance component of an example frequency response of electrochemical impedance sensors, according to some implementations.

FIG. 15-13B shows the imaginary (Z″) impedance component of an example frequency response of electrochemical impedance sensors, according to some implementations.

FIG. 15-14A shows an example baseline frequency response and an example frequency response to hydrogen peroxide, according to some implementations.

FIG. 15-14B shows example frequency responses to acetone and water, according to some implementations.

FIG. 15-14C shows example frequency responses to ethanol and ammonia, according to some implementations.

FIG. 16 illustrates a flowchart of a method for biomarker detection using functionalized bioFETs, according to aspects of the present disclosure.

FIG. 17 illustrates a flowchart for a biomarker detection method using functionalized bioFETs, according to an embodiment.

FIG. 18 illustrates a flowchart of a method for biomarker detection using functionalized bioFETs, according to aspects of the present disclosure.

FIG. 19 illustrates a flowchart of a method for early-stage cancer biomarker detection, according to an embodiment.

FIG. 20 illustrates a flowchart of a cancer detection system, according to aspects of the present disclosure.

FIG. 21 illustrates a block diagram of a bioFET functionalization system, according to an embodiment.

FIG. 22 illustrates an orthogonal view of a biosensor system for biomarker detection, according to aspects of the present disclosure.

FIG. 23 illustrates a schematic view of an in-field rapid detection bioFET sensor system, according to an embodiment.

### DETAILED DESCRIPTION

The present disclosure relates to the field of biosensor technology, specifically focusing on portable and field-deployable systems for detecting and quantifying biological analytes in liquid samples. This area of technology has applications in healthcare diagnostics, environmental monitoring, food safety testing, scientific research, etc.

Current biosensing technologies often require complex laboratory equipment, specialized personnel, and time-consuming procedures, limiting their use in field or point-of-care settings. Existing portable biosensor systems face challenges in achieving the sensitivity, specificity, and reliability of laboratory-based methods while maintaining a compact form factor suitable for on-site analysis. Additionally, many current systems struggle with issues related to sensor stability, cross-reactivity with interfering substances, and the ability to detect multiple analytes simultaneously.

The present disclosure introduces an automated portable biosensor system that utilizes a vertical graphene field effect transistor (FET) array to overcome these limitations. By combining three-dimensional graphene-based sensing elements with integrated microfluidics and automated measurement capabilities, the system achieves high sensitivity and specificity in a compact, field-deployable format. The vertical graphene structure provides increased surface area for analyte binding, while the automated sample handling and measurement processes ensure consistent and reliable results without the need for specialized operators.

Additionally, the disclosed system incorporates advanced features such as current drift correction, programmable voltage sweeping, and sensor performance validation to enhance measurement accuracy and reliability. The modular design, consisting of a separate sensor element and electronic control module, allows for easy customization and upgrades to detect different analytes. This versatility, combined with the system's ability to perform rapid, on-site analysis of liquid samples, makes it suitable for a wide range of applications where timely and accurate biosensing is critical.

Taking a step back, the integration of vertical graphene within a graphene field-effect transistor (GFET) represents a groundbreaking advancement in biosensor technology that has not been previously realized. This novel approach leverages the unique properties of vertical graphene structures to overcome limitations inherent in traditional monolayer graphene systems. The three-dimensional nature of vertical graphene provides a significantly larger surface area for binding sites, potentially increasing the sensor's sensitivity and detection capabilities. Moreover, the vertical orientation may help mitigate charge screening effects that often plague planar graphene sensors, especially in ionic solutions. By extending into the sample solution, vertical graphene structures may allow for more efficient interaction with target analytes beyond the Debye screening length.

Further, the methods disclosed herein may allow for growing vertical graphene in-situ directly on the sensor itself, which is in stark contrast to prior art systems. This innovative approach potentially eliminates the need for complex transfer processes typically associated with graphene-based sensors. By enabling direct growth on the sensor substrate, the method may provide several advantages over conventional techniques. The in-situ growth process may result in better adhesion and electrical contact between the vertical graphene structures and the underlying electrodes, potentially improving sensor performance and reliability. Additionally, this approach may allow for precise control over the graphene morphology and distribution across the sensor surface, tailoring the sensing capabilities to specific applications. The ability to grow vertical graphene directly on the sensor may also simplify manufacturing processes, potentially reducing production costs and increasing scalability. This direct integration of vertical graphene growth with sensor fabrication represents a significant departure from prior art systems, which often rely on separate graphene synthesis and transfer steps.

As such, the innovative combination of vertical graphene with GFET architecture, as disclosed hereinbelow, may enable the development of highly sensitive, specific, and robust biosensors capable of detecting analytes at lower concentrations and with greater accuracy than previously possible with monolayer graphene systems.

As has been discussed, the present disclosure relates to the field of biosensor technology, specifically focusing on portable and field-deployable systems for detecting and quantifying biological analytes in liquid samples. This area of technology has applications in healthcare diagnostics, environmental monitoring, food safety testing, and scientific research, with a particular emphasis on early cancer detection and diagnosis.

Current biosensing technologies often require complex laboratory equipment, specialized personnel, and time-consuming procedures, limiting their use in field or point-of-care settings. Existing portable biosensor systems face challenges in achieving the sensitivity, specificity, and reliability of laboratory-based methods while maintaining a compact form factor suitable for on-site analysis. Additionally, many current systems struggle with issues related to sensor stability, cross-reactivity with interfering substances, and the ability to detect multiple analytes simultaneously.

The present disclosure also introduces an automated portable biosensor system that utilizes a vertical graphene field effect transistor (FET) array to overcome these limitations. By combining three-dimensional graphene-based sensing elements with integrated microfluidics and automated measurement capabilities, the system achieves high sensitivity and specificity in a compact, field-deployable format. The vertical graphene structure provides increased surface area for analyte binding, while the automated sample handling and measurement processes ensure consistent and reliable results without the need for specialized operators.

Additionally, the disclosed system incorporates advanced features such as current drift correction, programmable voltage sweeping, and sensor performance validation to enhance measurement accuracy and reliability. The modular design, consisting of a separate sensor element and electronic control module, allows for easy customization and upgrades to detect different analytes. This versatility, combined with the system's ability to perform rapid, on-site analysis of liquid samples, makes it suitable for a wide range of applications where timely and accurate biosensing is critical, including but not limited to cancer diagnosis and monitoring of other diseases.

### Definitions and Use of Figures

Some of the terms used in this description are defined below for easy reference. The presented terms and their respective definitions are not rigidly restricted to these definitions-a term may be further defined by the term's use within this disclosure. The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application and the appended claims, the term “or” is intended to mean an inclusive “or” rather than an exclusive “or”. That is, unless specified otherwise, or is clear from the context, “X employs A or B” is intended to mean any of the natural inclusive permutations. That is, if X employs A, X employs B, or X employs both A and B, then “X employs A or B” is satisfied under any of the foregoing instances. As used herein, at least one of A or B means at least one of A, or at least one of B, or at least one of both A and B. In other words, this phrase is disjunctive. The articles “a” and “an” as used in this application and the appended claims should generally be construed to mean “one or more” unless specified otherwise or is clear from the context to be directed to a singular form.

Various embodiments are described herein with reference to the figures. It should be noted that the figures are not necessarily drawn to scale, and that elements of similar structures or functions are sometimes represented by like reference characters throughout the figures. It should also be noted that the figures are only intended to facilitate the description of the disclosed embodiments-they are not representative of an exhaustive treatment of all possible embodiments, and they are not intended to impute any limitation as to the scope of the claims. In addition, an illustrated embodiment need not portray all aspects or advantages of usage in any particular environment.

An aspect or an advantage described in conjunction with a particular embodiment is not necessarily limited to that embodiment and can be practiced in any other embodiments even if not so illustrated. References throughout this specification to “some embodiments” or “other embodiments” refer to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments. The disclosed embodiments are not intended to be limiting of the claims.

Within the context of the present disclosure, “vertical graphene” refers to a three-dimensional graphene structure.

### DESCRIPTIONS OF EXEMPLARY EMBODIMENTS

The following description sets forth exemplary aspects of the present disclosure. It should be recognized, however, that such description is not intended as a limitation on the scope of the present disclosure. Rather, the description also encompasses combinations and modifications to those exemplary aspects described herein.

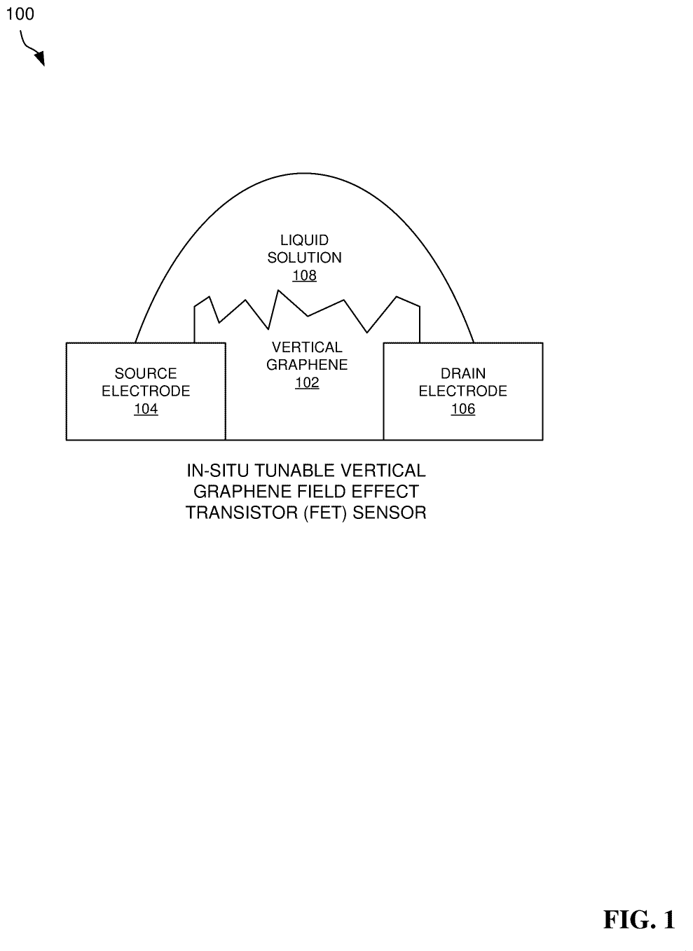

FIG. 1 illustrates a cross-sectional view of a sensor system 100, in accordance with one embodiment. As an option, the sensor system 100 may be implemented in the context of any one or more of the embodiments set forth in any previous and/or subsequent Figures and/or description thereof. Of course, however, the sensor system 100 may be implemented in the context of any desired environment. Further, the aforementioned definitions may equally apply to the description below.

The sensor system 100 includes vertical graphene 102 positioned between a source electrode 104 and a drain electrode 106. A liquid solution 108 is shown positioned above the vertical graphene 102. The vertical graphene 102 extends upward from the surface between the source electrode 104 and drain electrode 106, providing a three-dimensional sensing structure.

In some cases, the vertical graphene 102 may have a thickness ranging from 300 to 500 nanometers. This thickness range may allow for optimal interaction with analytes present in the liquid solution 108 while maintaining structural integrity and electrical properties.

The vertical graphene 102 may exhibit a distinct morphology characterized by a jagged and rough surface extending upward from the substrate between the source electrode 104 and drain electrode 106. This three-dimensional structure of the vertical graphene 102 creates a significantly larger surface area compared to conventional flat graphene layers.

The irregular surface of the vertical graphene 102 forms numerous protrusions, edges, and cavities along its surface. This complex topography may provide an abundance of potential binding sites for analytes present in the liquid solution 108. The increased number of binding sites may enhance the sensor's sensitivity and detection capabilities.

Additionally, the vertical orientation and rough surface of the graphene 102 may help mitigate charge screening effects that often limit the performance of planar graphene sensors, especially in ionic solutions. By extending into the liquid solution 108, the vertical graphene 102 may allow for more efficient interaction with target analytes beyond the Debye screening length, potentially overcoming a common limitation in conventional graphene field-effect transistors.

Further, the jagged morphology of the vertical graphene 102 may also create localized regions of high curvature and defect sites, which can exhibit enhanced electrochemical properties and reactivity. These features may further contribute to the sensor's performance by providing additional active sites for analyte interaction and charge transfer.

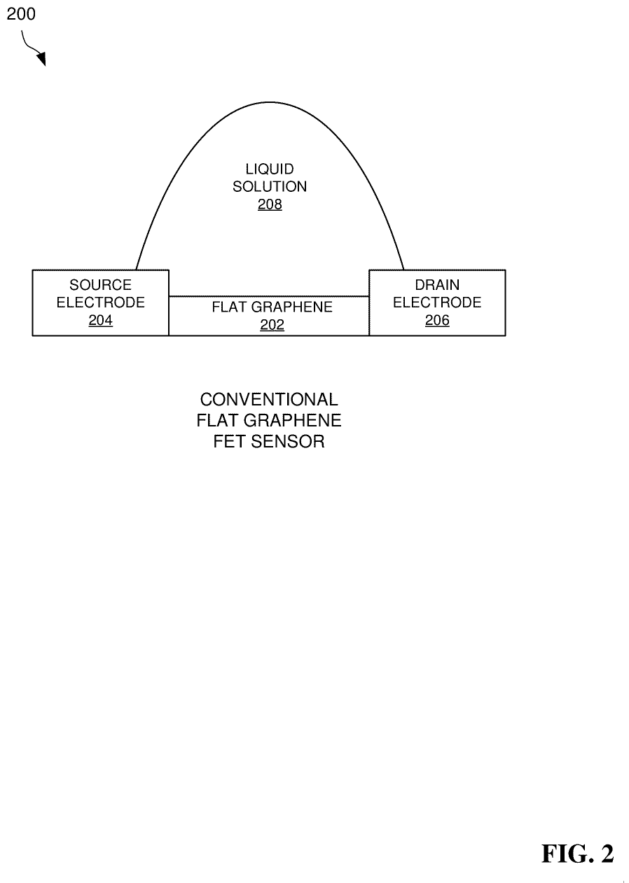

In contrast to monolayer graphene systems (such as shown in FIG. 2), the vertical graphene 102 structure may offer superior performance in terms of both increased binding capacity and reduced charge screening effects, potentially enabling more sensitive and reliable detection of analytes in liquid samples.

The source electrode 104 and drain electrode 106 are arranged on opposite sides of the vertical graphene 102 to enable electrical measurements. These electrodes may be composed of conductive materials such as metals or highly doped semiconductors, allowing for efficient charge transfer between the vertical graphene 102 and external measurement circuitry.

The liquid solution 108 interfaces with the vertical graphene 102 to enable detection of analytes present in the solution. As the liquid solution 108 comes into contact with the vertical graphene 102, analytes within the solution may interact with the graphene surface, potentially altering its electrical properties in a measurable way.

Additionally, the vertical graphene 102 may be functionalized with specific chemical groups or biomolecules to enhance selectivity towards particular analytes. This functionalization may involve covalent or non-covalent modification of the graphene surface to introduce binding sites for target molecules.

In some cases, the sensor system 100 may incorporate multiple vertical graphene structures arranged in an array format. This configuration may allow for simultaneous detection of multiple analytes or improved signal-to-noise ratio through redundant measurements.

The cross-sectional view provided by FIG. 1 demonstrates how the vertical orientation of the graphene 102 may overcome limitations associated with traditional planar graphene sensors. By extending into the liquid solution 108, the vertical graphene 102 may provide increased surface area for analyte interaction and potentially mitigate issues related to the Debye screening length in ionic solutions.

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described.

FIG. 2 illustrates a conventional flat graphene FET sensor system 200, in accordance with one embodiment. As an option, the conventional flat graphene FET sensor system 200 may be implemented in the context of any one or more of the embodiments set forth in any previous and/or subsequent Figures and/or description thereof. Of course, however, the conventional flat graphene FET sensor system 200 may be implemented in the context of any desired environment. Further, the aforementioned definitions may equally apply to the description below.

FIG. 2 illustrates a conventional flat graphene FET sensor system 200, which is presented to provide context for appreciating the novelty of the vertical graphene sensor system shown in FIG. 1. While this configuration of FIG. 2 has been widely used in biosensing applications, it faces limitations in terms of available surface area for analyte interactions and susceptibility to charge screening effects in ionic solutions, as well as other common issues. Understanding these constraints helps highlight the advantages offered by the vertical graphene structure presented in FIG. 1, which addresses many of the shortcomings inherent in conventional flat graphene FET sensors. As such, the discussion herein, as it relates to FIG. 2, is include merely as context for more fully understanding the novelty of FIG. 1.

The conventional flat graphene FET sensor system 200 includes a flat graphene layer 202 positioned between a source electrode 204 and a drain electrode 206. A liquid solution 208 is shown positioned above the flat graphene layer 202. The liquid solution 208 forms an arc or dome shape over the flat graphene 202 between the source electrode 204 and drain electrode 206.

In contrast to the vertical graphene 102 of the sensor system 100 shown in FIG. 1, the flat graphene layer 202 lies parallel to the substrate surface as a thin layer. This planar configuration may result in a smaller surface area available for interaction with the liquid solution 208 compared to the three-dimensional structure of the vertical graphene 102. The flat graphene layer 202 may have a thickness of only a few atomic layers, typically ranging from 1 to 10 layers of graphene. This ultra-thin structure may make the sensor more susceptible to environmental factors and potential damage.

In some cases, the flat graphene layer 202 may be functionalized with specific chemical groups or biomolecules to enhance selectivity towards particular analytes. However, the limited surface area of the flat graphene layer 202 may restrict the number of functional groups that can be attached compared to the vertical graphene 102. The liquid solution 208 interacts with the flat graphene layer 202 primarily at the top surface of the graphene. This limited interaction area may result in reduced sensitivity compared to the vertical graphene sensor system 100, where the liquid solution 108 can penetrate between the vertical graphene structures.

In various embodiments, the flat graphene layer 202 may be grown or transferred onto various substrate materials, such as silicon dioxide or flexible polymers.

In various embodiments, the conventional flat graphene FET sensor system 200 may be integrated into array formats, allowing for simultaneous detection of multiple analytes. However, the planar nature of the flat graphene layer 202 may limit the density of sensing elements compared to vertical graphene arrays.

Taking a step back, FIG. 1 illustrates a sensor system 100 utilizing vertical graphene 102, while FIG. 2 depicts a conventional flat graphene FET sensor system 200. The vertical graphene 102 in FIG. 1 may be grown in-situ directly on the sensor substrate, potentially resulting in a higher purity and more seamless integration with the underlying electrodes. This in-situ growth process may significantly enhance the graphene's ability to function as a sensor by minimizing contamination and preserving its intrinsic properties. In contrast, the flat graphene layer 202 in FIG. 2 is typically produced separately and transferred onto the sensor substrate, a process that may introduce impurities, defects, or residual transfer materials. The in-situ growth of vertical graphene 102 may also allow for better control over the graphene morphology and distribution, potentially leading to more consistent and reliable sensor performance compared to the transferred flat graphene 202 in conventional systems.

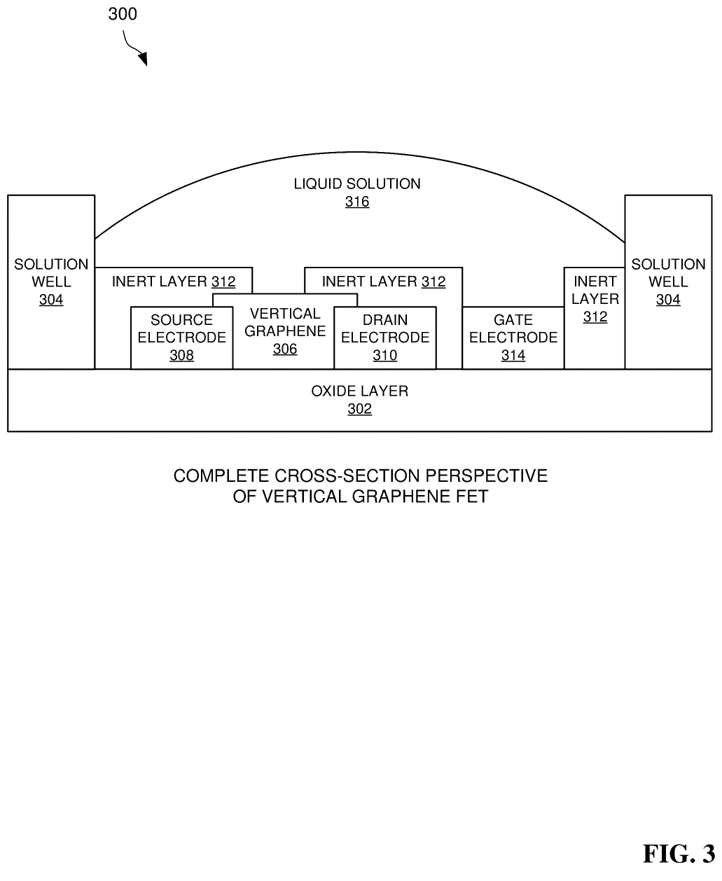

FIG. 3 illustrates a complete cross-section perspective view of a vertical graphene field effect transistor (FET) sensor system 300, in accordance with one embodiment. As an option, the vertical graphene field effect transistor (FET) sensor system 300 may be implemented in the context of any one or more of the embodiments set forth in any previous and/or subsequent Figures and/or description thereof. Of course, however, the vertical graphene field effect transistor (FET) sensor system 300 may be implemented in the context of any desired environment. Further, the aforementioned definitions may equally apply to the description below.

The sensor system 300 includes an oxide layer 302 as a base substrate. Two solution wells 304 are positioned at opposite ends of the sensor system 300. A vertical graphene structure 306 is disposed between the solution wells 304. The vertical graphene 306 is electrically connected to a source electrode 308 and a drain electrode 310. An inert layer 312 is disposed between and around the electrodes and vertical graphene structure. A gate electrode 314 is positioned adjacent to the vertical graphene 306 and drain electrode 310. The sensor system 300 is configured to contain a liquid solution 316 that can flow across the vertical graphene 306 between the solution wells 304.

The oxide layer 302 may serve as an insulating foundation for the sensor system 300. In some cases, the oxide layer 302 may be composed of silicon dioxide or another suitable dielectric material. The oxide layer 302 may provide electrical isolation between the various components of the sensor system 300 and any underlying substrate or circuitry.

The solution wells 304 may be designed to hold and control the flow of the liquid solution 316 across the vertical graphene 306. In some cases, the solution wells 304 may be fabricated using photolithography techniques to create precise dimensions and shapes. The solution wells 304 may be connected to external microfluidic systems for automated sample introduction and removal.

The vertical graphene 306 may be grown using plasma-enhanced chemical vapor deposition (PECVD) techniques. This growth method may allow for precise control over the morphology and density of the vertical graphene structures. The PECVD process may involve the use of specific gas mixtures and plasma conditions to promote vertical growth of graphene flakes from the substrate surface.

The source electrode 308 and drain electrode 310 may be composed of conductive materials such as metals or highly doped semiconductors. In some cases, these electrodes may be patterned using photolithography and deposition techniques to ensure precise placement and dimensions. The electrical connections between the vertical graphene 306 and the source electrode 308 and drain electrode 310 may be used for the operation of the field effect transistor.

The inert layer 312 may serve to protect and isolate certain regions of the sensor system 300. In some cases, the inert layer 312 may be composed of materials such as silicon nitride or aluminum oxide. The inert layer 312 may help prevent unwanted electrical interactions between components and may also provide chemical resistance to protect the underlying structures.

The gate electrode 314 may be used to modulate the electrical properties of the vertical graphene 306. In some cases, the gate electrode 314 may be positioned to maximize its influence on the vertical graphene 306 while minimizing interference with the liquid solution 316 flow. The gate electrode 314 may be connected to external control circuitry to allow for precise adjustment of the sensor system 300 sensitivity and response.

The liquid solution 316 may contain the analytes of interest for detection by the sensor system 300. In some cases, the composition of the liquid solution 316 may be carefully controlled to optimize sensor performance. The flow of the liquid solution 316 across the vertical graphene 306 may be regulated to ensure consistent and reproducible sensor readings.

In various embodiments, the solution wells 304 may provide containment and flow control of the liquid solution 316 as it interacts with the vertical graphene 306. This controlled interaction may allow for precise measurements of changes in the electrical properties of the vertical graphene 306 in response to analyte binding.

The vertical orientation of the graphene 306 in the sensor system 300 may provide several advantages over traditional planar graphene sensors. The increased surface area of the vertical graphene 306 may allow for more efficient interaction with analytes in the liquid solution 316. Additionally, the vertical structure may help overcome limitations related to the Debye screening length in ionic solutions, potentially improving sensor sensitivity and response time.

In various embodiments, the dimensions and spacing of the vertical graphene 306 structures may be optimized for specific sensing applications. For example, the height, density, and distribution of the vertical graphene 306 may be tailored to enhance sensitivity to particular analytes or to improve the signal-to-noise ratio of the sensor system 300.

In various embodiments, the sensor system 300 may incorporate multiple vertical graphene 306 structures arranged in an array format. This configuration may allow for simultaneous detection of multiple analytes or improved signal reliability through redundant measurements. The array format may also enable spatial mapping of analyte concentrations across the sensor surface.

In various embodiments, the vertical graphene 306 may be functionalized with specific chemical groups or biomolecules to enhance selectivity towards particular analytes. This functionalization may involve covalent or non-covalent modification of the graphene surface to introduce binding sites for target molecules. The three-dimensional nature of the vertical graphene 306 may allow for a higher density of functional groups compared to planar graphene sensors.

It is to be appreciated that FIG. 3 illustrates one example of a detailed configuration for a possible arrangement of the vertical graphene FET system 300. Other possible configurations of the vertical graphene FET system are envisioned.

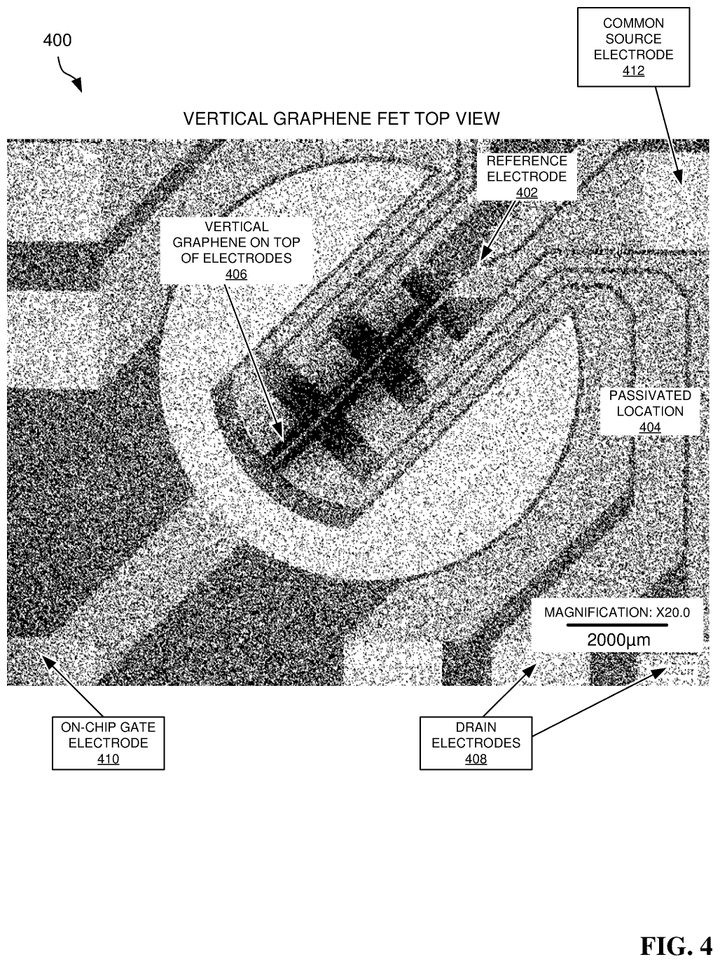

FIG. 4 illustrates a top view of a sensor assembly 400, in accordance with one embodiment. As an option, the sensor assembly 400 may be implemented in the context of any one or more of the embodiments set forth in any previous and/or subsequent Figures and/or description thereof. Of course, however, the sensor assembly 400 may be implemented in the context of any desired environment. Further, the aforementioned definitions may equally apply to the description below.

The sensor assembly 400 includes vertical graphene 406 positioned on top of electrodes. A reference electrode 402 is provided for electrical measurements. The sensor assembly 400 comprises drain electrodes 408 and a source electrode 412 positioned beneath the vertical graphene 406. A gate electrode 410 is included for controlling the electrical characteristics of the device. A passivated location 404 is shown, which helps isolate certain regions of the sensor assembly 400.

The vertical graphene 406 may be arranged to interface with the electrodes while maintaining exposure to the measurement environment. In some cases, the vertical graphene 406 may be grown directly on the electrodes (such as by using plasma-enhanced chemical vapor deposition techniques). The three-dimensional structure of the vertical graphene 406 may provide increased surface area for analyte interaction compared to planar graphene sensors.

The reference electrode 402 may be used to establish a stable reference potential for electrical measurements. In some cases, the reference electrode 402 may be composed of materials such as silver/silver chloride or platinum. The reference electrode 402 may be positioned to minimize interference with the sample flow while maintaining electrical contact with the liquid solution.

The drain electrodes 408 and source electrode 412 may be arranged to enable field effect transistor operation with the vertical graphene 406. In some cases, these electrodes may be patterned using photolithography techniques to ensure precise placement and dimensions. The arrangement of multiple drain electrodes 408 may allow for differential measurements or redundancy in sensor readings.

The gate electrode 410 may be used to modulate the electrical properties of the vertical graphene 406. In some cases, the gate electrode 410 may be positioned to maximize its influence on the vertical graphene 406 while minimizing interference with sample flow. The gate electrode 410 may be connected to external control circuitry to allow for precise adjustment of the sensor assembly 400 sensitivity and response.

The passivated location 404 may help isolate certain regions of the sensor assembly 400. In some cases, the passivated location 404 may be composed of materials such as silicon dioxide or silicon nitride. The passivation may prevent unwanted electrical interactions and protect sensitive components from the liquid environment.

The arrangement of components in the sensor assembly 400 may allow for efficient interaction between the vertical graphene 406 and the liquid sample while maintaining stable electrical connections. In various embodiments, the dimensions and spacing of the electrodes in the sensor assembly 400 may be optimized for specific sensing applications. For example, the width and gap between the drain electrodes 408 and source electrode 412 may be tailored to enhance sensitivity to particular analytes or to improve the signal-to-noise ratio of the sensor assembly 400.

In various embodiments, the sensor assembly 400 may incorporate multiple vertical graphene 406 regions arranged in an array format. This configuration may allow for simultaneous detection of multiple analytes or improved signal reliability through redundant measurements. The array format may also enable spatial mapping of analyte concentrations across the sensor surface.

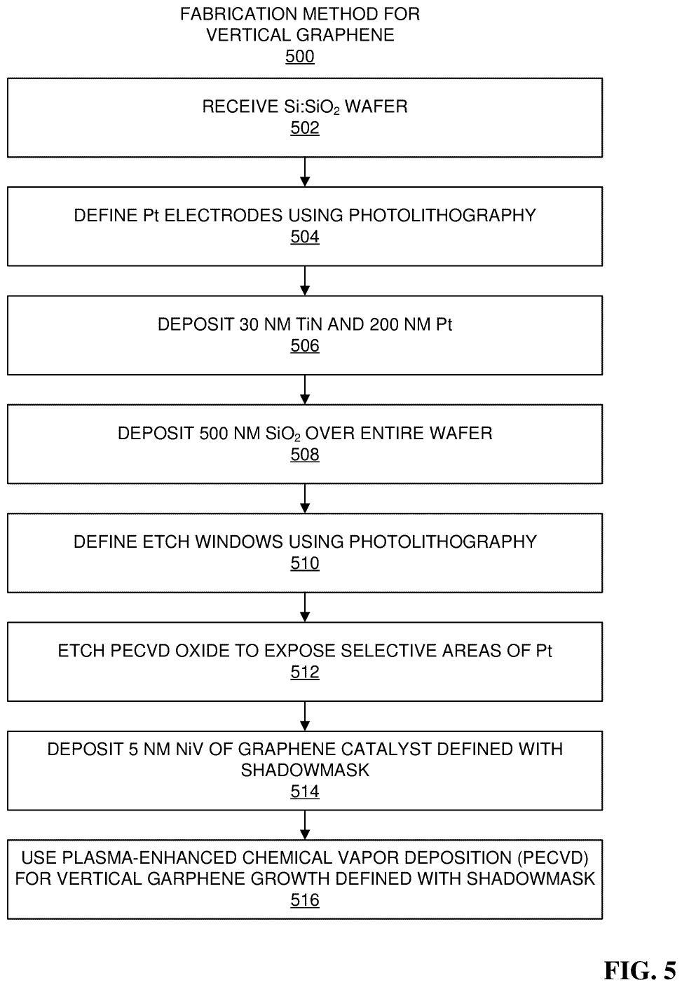

FIG. 5 illustrates a flowchart of a method 500 for fabricating vertical graphene, in accordance with one embodiment. As an option, the method 500 may be implemented in the context of any one or more of the embodiments set forth in any previous and/or subsequent Figures and/or description thereof. Of course, however, the method 500 may be implemented in the context of any desired environment. Further, the aforementioned definitions may equally apply to the description below.

The method 500 begins at a step 502 with receiving a Si:SiO2 wafer. The method 500 proceeds to a step 504, where Pt electrodes are defined using photolithography. At a step 506, the method 500 includes depositing 30 nm TIN and 200 nm Pt. The method 500 continues to a step 508, where 500 nm SiO2 is deposited over the entire wafer.

In operation 502, the Si:SiO2 wafer may serve as the substrate for the vertical graphene fabrication process. The Si:SiO2 wafer may provide a suitable surface for subsequent deposition and growth steps. In some cases, the Si:SiO2 wafer may be cleaned and prepared prior to use to ensure a contamination-free surface. The thickness and quality of the SiO2 layer on the Si wafer may be carefully controlled to optimize the subsequent fabrication steps. In some cases, the SiO2 layer may be thermally grown or deposited using techniques such as chemical vapor deposition to achieve the desired properties.

In operation 504, photolithography may be used to define the patterns for the Pt electrodes on the Si:SiO2 wafer. This process may involve applying a photoresist layer, exposing it to light through a mask with the desired electrode pattern, and developing the photoresist to create openings where the Pt electrodes will be deposited. The photolithography process may be optimized to achieve high resolution and precise alignment of the electrode patterns. In some cases, multiple photolithography steps may be employed to create complex electrode configurations or to integrate additional features into the device structure.

In operation 506, the deposition of 30 nm TIN and 200 nm Pt may be performed using techniques such as sputtering or electron beam evaporation. The TiN layer may serve as an adhesion layer to improve the bonding between the Pt electrodes and the underlying Si:SiO2 substrate. The thickness and uniformity of the TIN and Pt layers may be carefully controlled to ensure optimal electrical properties and stability of the electrodes. In some cases, post-deposition treatments such as annealing may be performed to improve the quality and performance of the deposited layers.

In operation 508, a 500 nm SiO2 layer may be deposited over the entire wafer using techniques such as plasma-enhanced chemical vapor deposition (PECVD). This SiO2 layer may serve as an insulating and protective layer for the underlying electrode structures. The deposition conditions for the SiO2 layer may be optimized to achieve the desired thickness, density, and dielectric properties. In some cases, the SiO2 layer may be planarized or subjected to additional treatments to improve its surface quality and compatibility with subsequent processing steps.

At a step 510, the method 500 includes defining etch windows using photolithography. The method 500 then moves to a step 512, where PECVD oxide is etched to expose selective areas of Pt. At a step 514, the method 500 includes depositing 5 nm NiV of graphene catalyst defined with shadowmask. The method 500 concludes at a step 516 with using plasma-enhanced chemical vapor deposition (PECVD) for vertical graphene growth defined with shadowmask.

In operation 510, photolithography may be used to define etch windows in the SiO2 layer deposited in step 508. This process may involve applying and patterning a photoresist layer to create openings where the underlying Pt electrodes will be exposed. The alignment and dimensions of the etch windows may be carefully controlled to ensure proper exposure of the desired Pt electrode areas.

In operation 512, the PECVD oxide may be etched using techniques such as reactive ion etching (RIE) or wet chemical etching to expose selective areas of the Pt electrodes. The etching process may be optimized to achieve high selectivity between the SiO2 and Pt layers, ensuring clean and well-defined openings. The etching parameters, such as gas composition, power, and duration, may be carefully controlled to achieve the desired etch profile and minimize damage to the underlying Pt electrodes. In some cases, a multi-step etching process may be employed to improve the quality and uniformity of the exposed Pt surfaces.

In operation 514, a 5 nm NiV graphene catalyst layer may be deposited using techniques such as sputtering or electron beam evaporation. The shadowmask may be used to define the areas where the catalyst will be deposited, allowing for selective growth of vertical graphene in subsequent steps. The composition and thickness of the NiV catalyst layer may be optimized to promote the growth of high-quality vertical graphene structures. In some cases, the catalyst layer may be subjected to post-deposition treatments such as annealing to improve its catalytic activity and uniformity.

In operation 516, PECVD may be used for vertical graphene growth, with the growth areas defined by the shadowmask. The PECVD process may involve the use of specific gas mixtures, plasma conditions, and temperature profiles to promote the vertical growth of graphene structures from the NiV catalyst layer. The PECVD growth parameters may be carefully optimized to control the morphology, density, and height of the vertical graphene structures. In some cases, in-situ monitoring techniques may be employed to track the growth process and ensure consistent and reproducible results.

The method 500 exemplifies a resolution to challenges in fabricating high-quality vertical graphene structures for sensing applications. By combining precise electrode patterning, controlled deposition of catalyst layers, and optimized PECVD growth conditions, the method 500 may enable the production of vertical graphene-based devices with enhanced surface area and electrical properties.

The integration of shadowmask techniques for catalyst deposition and graphene growth may allow for selective patterning of vertical graphene structures without the need for additional etching or transfer steps. This approach may help preserve the integrity of the underlying electrode structures and improve the overall device performance.

In various embodiments, the method 500 may be modified to incorporate additional processing steps or alternative techniques for specific applications. For example, the electrode materials may be varied to include other conductive materials such as gold or graphene itself, potentially altering the electrical characteristics of the final device.

In various embodiments, the catalyst composition and deposition method may be adjusted to tailor the properties of the resulting vertical graphene structures. For instance, different metal or alloy catalysts may be explored to promote specific graphene morphologies or to enhance the growth rate and quality of the vertical structures.

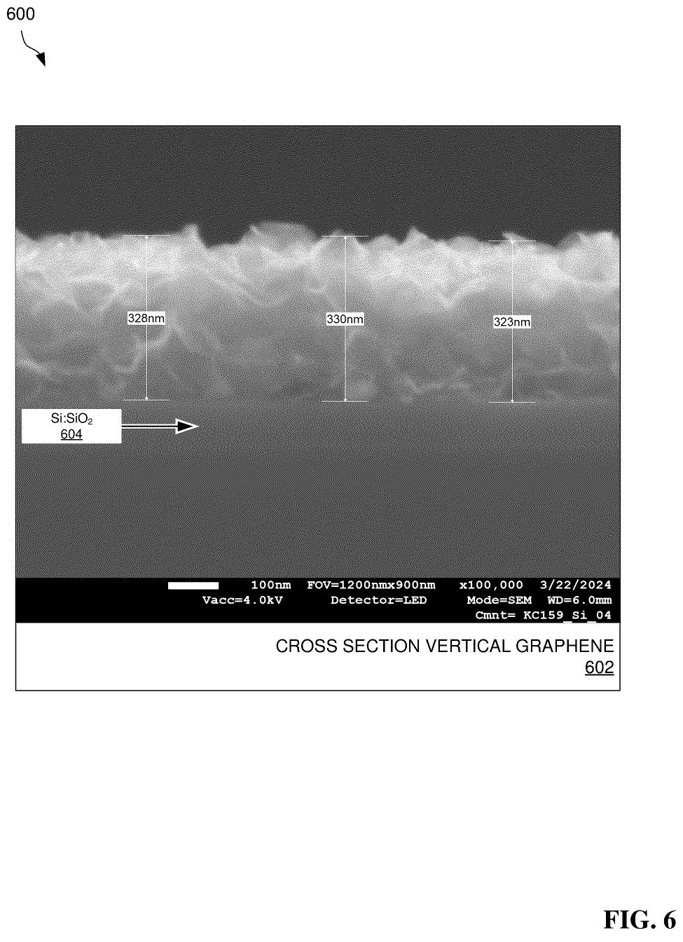

FIG. 6 illustrates a cross-sectional view of a sensor system 600, in accordance with one embodiment. As an option, the sensor system 600 may be implemented in the context of any one or more of the embodiments set forth in any previous and/or subsequent Figures and/or description thereof. Of course, however, the sensor system 600 may be implemented in the context of any desired environment. Further, the aforementioned definitions may equally apply to the description below.

The sensor system 600 includes a cross section vertical graphene 602 formed on a silicon dioxide substrate 604. The cross section vertical graphene 602 exhibits a non-uniform surface morphology with varying heights ranging from approximately 323 nm to 330 nm as measured from the silicon dioxide substrate 604.

The cross section vertical graphene 602 demonstrates a flake-like structure with interconnected graphene layers extending upward from the silicon dioxide substrate 604. In some cases, the flake-like structure may provide increased surface area for interaction with analytes in a liquid sample. The non-uniform surface morphology of the cross section vertical graphene 602 may contribute to enhanced sensing capabilities by creating a variety of binding sites for target molecules.

The varying heights of the cross section vertical graphene 602 may result from the growth process used to form the vertical graphene structures. In some cases, the height variations may be controlled by adjusting growth parameters such as temperature, gas composition, and plasma conditions during the fabrication process. The ability to control the height and morphology of the vertical graphene may allow for optimization of the sensor system 600 for specific sensing applications.

The silicon dioxide substrate 604 provides a foundation for the vertical graphene growth and electrical isolation. In some cases, the silicon dioxide substrate 604 may be selected for its compatibility with standard semiconductor fabrication processes and its ability to withstand the high temperatures often used in vertical graphene growth. The thickness and quality of the silicon dioxide substrate 604 may be carefully controlled to ensure optimal electrical and mechanical properties of the sensor system 600.

The cross-sectional view reveals the three-dimensional nature of the vertical graphene 602 structure, showing how the graphene extends vertically from the planar silicon dioxide substrate 604 surface. This vertical orientation may provide several advantages over traditional planar graphene sensors, including increased surface area for analyte interaction and potential improvements in sensitivity and response time.

The sensor system 600 exemplifies a resolution to challenges in creating high-surface-area graphene structures for sensing applications. By utilizing vertical growth techniques on a silicon dioxide substrate, the sensor system 600 may achieve a significantly larger active sensing area compared to planar graphene sensors, potentially leading to improved sensitivity and detection limits for various analytes.

The non-uniform morphology and varying heights of the cross section vertical graphene 602 may also contribute to the sensor system's ability to detect a wide range of analyte sizes and types. The complex three-dimensional structure may create a variety of binding sites and interaction possibilities, potentially enabling multi-analyte detection or improved selectivity in certain sensing applications.

In various embodiments, the growth conditions for the cross section vertical graphene 602 may be modified to achieve specific morphologies or height distributions. For example, the plasma power, gas composition, or growth duration may be adjusted to promote the formation of denser or taller vertical graphene structures, potentially altering the sensing characteristics of the sensor system 600.

In various embodiments, the sensor system 600 may incorporate additional layers or structures between the silicon dioxide substrate 604 and the cross section vertical graphene 602. These intermediate layers may serve various functions, such as improving adhesion, modifying the electrical properties of the interface, or introducing additional functionalities to the sensor system 600. For example, a thin metal layer may be deposited on the silicon dioxide substrate 604 to serve as a bottom electrode or to enhance the catalytic activity for vertical graphene growth.

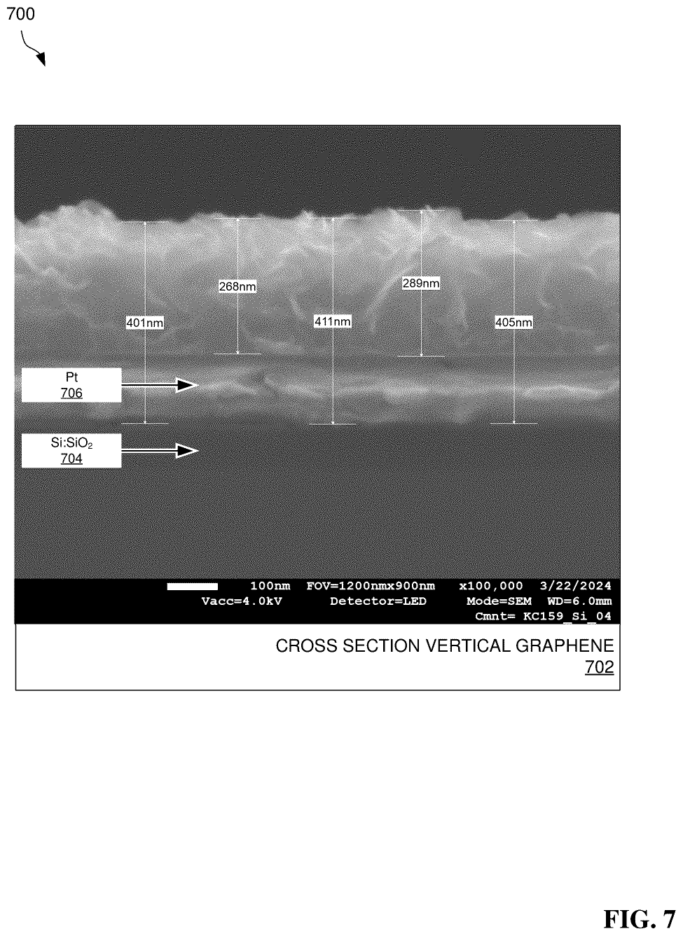

FIG. 7 illustrates a cross-sectional view of a sensor system 700, in accordance with one embodiment. As an option, the sensor system 700 may be implemented in the context of any one or more of the embodiments set forth in any previous and/or subsequent Figures and/or description thereof. Of course, however, the sensor system 700 may be implemented in the context of any desired environment. Further, the aforementioned definitions may equally apply to the description below.

The sensor system 700 includes a vertical graphene structure 702 formed on a silicon dioxide substrate 704. A platinum layer 706 is disposed on a portion of the silicon dioxide substrate 704. The vertical graphene 702 exhibits a non-uniform surface morphology with varying heights ranging from approximately 268 nm to 405 nm as measured at different points along the structure.

The vertical graphene 702 may be grown using plasma-enhanced chemical vapor deposition (PECVD) techniques. In some cases, the growth conditions may be optimized to achieve the desired height range and surface morphology. The non-uniform structure of the vertical graphene 702 may provide increased surface area for interaction with analytes in sensing applications.

The silicon dioxide substrate 704 may serve as an insulating foundation for the sensor system 700. In some cases, the thickness and quality of the silicon dioxide substrate 704 may be carefully controlled to ensure optimal electrical and mechanical properties of the overall device. The silicon dioxide substrate 704 may also provide compatibility with standard semiconductor fabrication processes.

The platinum layer 706 may function as an electrode contact for the sensor system 700. In some cases, the platinum layer 706 may be deposited using techniques such as sputtering or electron beam evaporation. The thickness and uniformity of the platinum layer 706 may be carefully controlled to ensure optimal electrical properties and stability.

The vertical graphene 702 is positioned adjacent to the platinum layer 706, with the platinum layer 706 serving as an electrode contact. This arrangement may allow for efficient electrical connection between the vertical graphene 702 and external measurement circuitry. In some cases, the interface between the vertical graphene 702 and the platinum layer 706 may be optimized to enhance charge transfer and reduce contact resistance.

The cross-sectional view reveals the interface between the vertical graphene 702 and the underlying silicon dioxide substrate 704, as well as the relative positioning of the platinum layer 706. This view may provide insights into the growth characteristics of the vertical graphene 702 and the quality of its adhesion to the substrate. In some cases, the interface between the vertical graphene 702 and the silicon dioxide substrate 704 may influence the electrical and mechanical properties of the sensor system 700.

In comparison to FIG. 6, the integration in FIG. 7 of the platinum layer as an electrode contact may provide advantages in terms of chemical stability and electrical conductivity. This configuration may enable more reliable and reproducible sensor measurements, potentially expanding the range of applications for graphene-based sensing devices.

In various embodiments, the composition of the electrode layer may be varied to include materials other than platinum. For instance, gold, palladium, or other noble metals may be explored as alternative electrode materials, each potentially offering unique advantages in terms of electrical properties, chemical stability, or compatibility with specific sensing applications.

In various embodiments, additional layers or structures may be incorporated between the silicon dioxide substrate 704 and the vertical graphene 702 or platinum layer 706. These intermediate layers may serve various functions, such as improving adhesion, modifying the electrical properties of the interfaces, or introducing additional functionalities to the sensor system 700. For example, a thin adhesion layer may be deposited beneath the platinum layer 706 to enhance its stability on the silicon dioxide substrate 704.

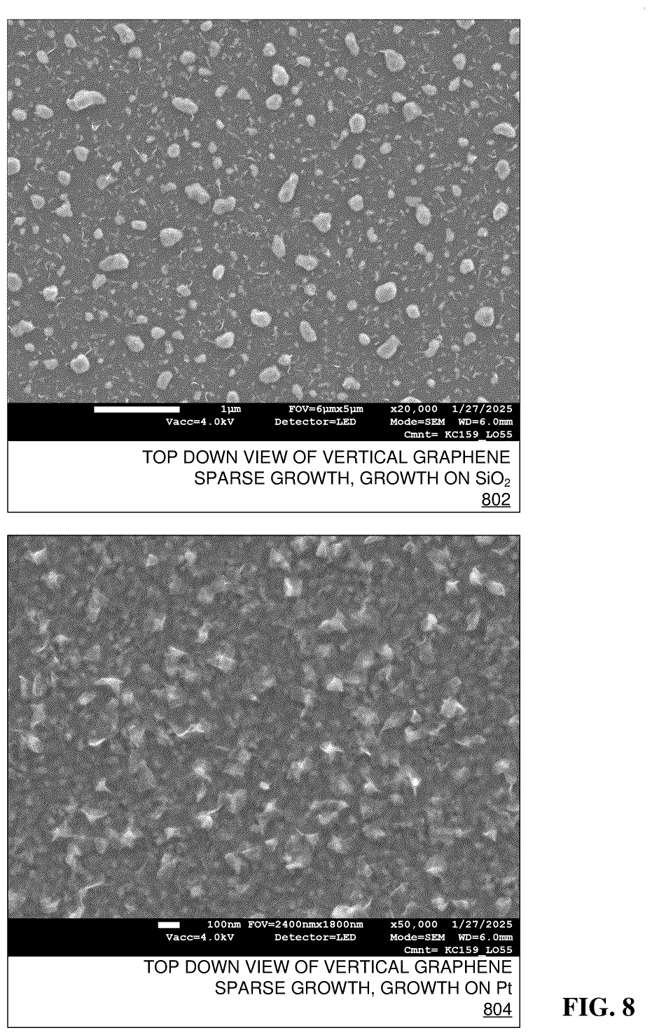

FIG. 8 illustrates a top-down view of vertical graphene structures grown on different substrates, in accordance with one embodiment. As an option, the vertical graphene structures may be implemented in the context of any one or more of the embodiments set forth in any previous and/or subsequent Figures and/or description thereof. Of course, however, the vertical graphene structures may be implemented in the context of any desired environment. Further, the aforementioned definitions may equally apply to the description below.

The figure shows two distinct vertical graphene growth patterns. A vertical graphene growth 802 shows a sparse distribution pattern of vertically-oriented graphene structures on a silicon dioxide substrate. A vertical graphene structure 804 demonstrates a similar sparse growth pattern but on a platinum substrate.

The vertical graphene growth 802 on the silicon dioxide substrate may exhibit a more uniform distribution compared to the vertical graphene structure 804 on the platinum substrate. In some cases, the silicon dioxide substrate may promote more consistent nucleation and growth of the vertical graphene structures across the surface.

The vertical graphene structure 804 on the platinum substrate may display a slightly denser growth pattern in certain areas compared to the vertical graphene growth 802 on silicon dioxide. This variation in density may be attributed to the different catalytic properties of platinum compared to silicon dioxide during the graphene growth process.

The vertical graphene formations appear as light-colored structures distributed across the darker substrate surfaces.

Both growth patterns exhibit a scattered arrangement of vertical graphene elements, though with slightly different morphologies depending on the underlying substrate material. The differences in morphology may be related to variations in surface energy, catalytic activity, and/or thermal properties between the silicon dioxide and platinum substrates.

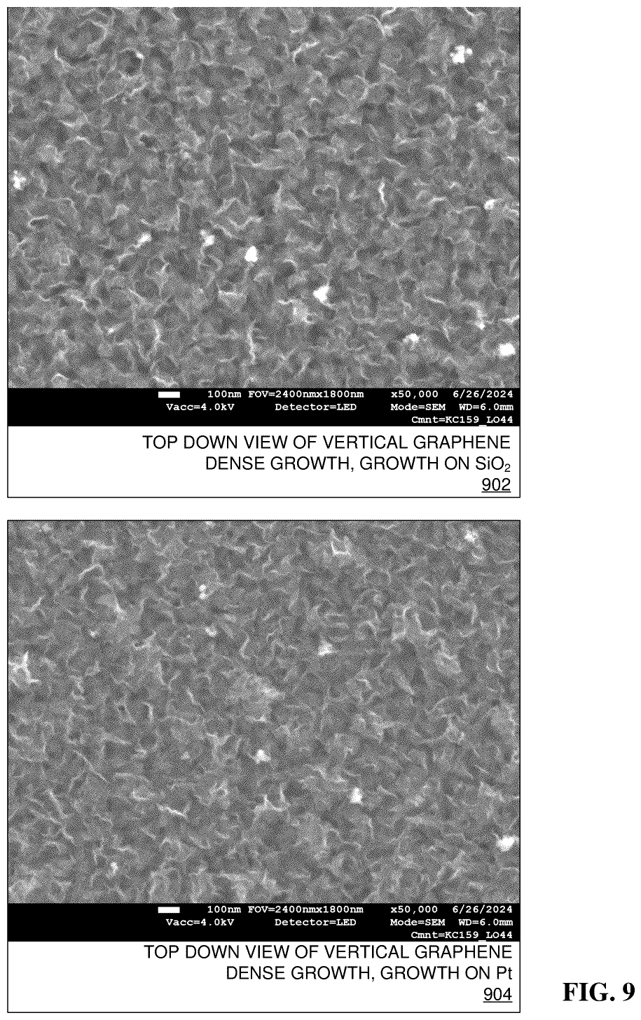

FIG. 9 illustrates a top-down view showing vertical graphene growth on different substrates, in accordance with one embodiment. As an option, the vertical graphene growth may be implemented in the context of any one or more of the embodiments set forth in any previous and/or subsequent Figures and/or description thereof. Of course, however, the vertical graphene growth may be implemented in the context of any desired environment. Further, the aforementioned definitions may equally apply to the description below.

The figure includes two comparative images showing a vertical graphene growth 902 on a silicon dioxide (SiO2) substrate and a vertical graphene growth 904 on a platinum (Pt) substrate. The vertical graphene growth 902 exhibits a dense, interconnected network structure with uniform coverage across the surface. The vertical graphene growth 904 demonstrates a similar growth pattern but with variations in the density and distribution of the graphene structures.

In some cases, the vertical graphene growth 902 on the silicon dioxide substrate may exhibit a more uniform distribution compared to the vertical graphene growth 904 on the platinum substrate. The silicon dioxide substrate may promote more consistent nucleation and growth of the vertical graphene structures across the surface due to its uniform surface properties and lack of catalytic activity. Additionally, the vertical graphene growth 904 on the platinum substrate may display regions of higher density compared to the vertical graphene growth 902 on silicon dioxide. This variation in density may be attributed to the catalytic properties of platinum during the graphene growth process, potentially leading to localized areas of enhanced growth.

Both vertical graphene growth 902 and vertical graphene growth 904 display characteristic three-dimensional morphology, though the underlying substrate influences the specific growth patterns and structural features observed in the top-down view.

In various embodiments, post-growth treatments may be applied to the vertical graphene structures to further modify their properties or enhance their sensing capabilities. These treatments may include chemical functionalization, plasma etching, or thermal annealing, potentially leading to substrate-specific optimization strategies for different sensing applications.

In various embodiments, the vertical graphene growth patterns observed on silicon dioxide and platinum substrates may be leveraged to create hybrid sensing devices. For instance, a single sensor chip may incorporate regions of vertical graphene grown on both substrate types, potentially enabling multi-modal sensing or improved selectivity through the combination of different graphene morphologies.

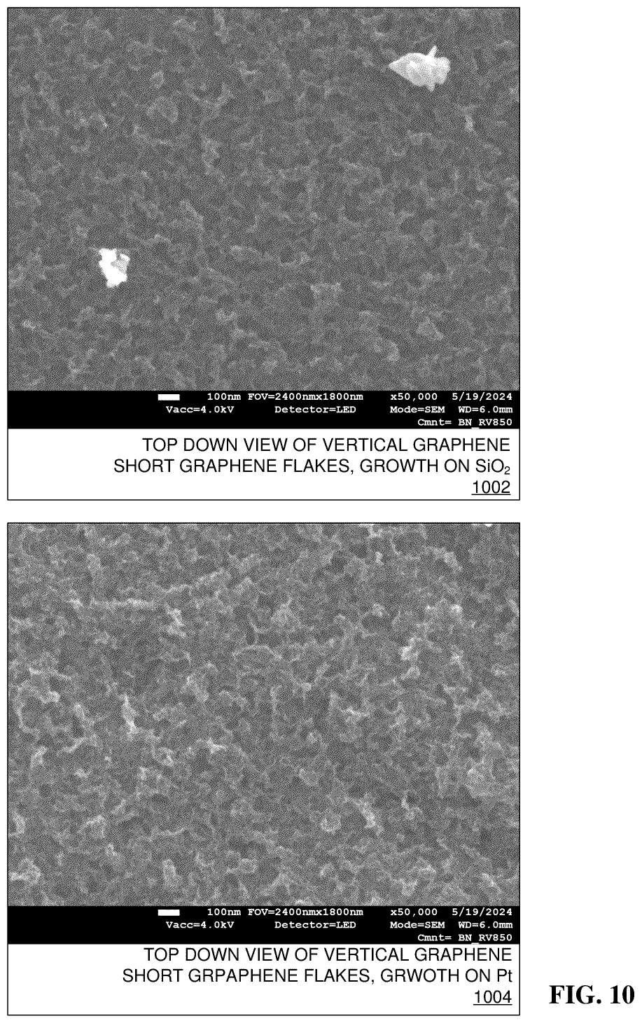

FIG. 10 illustrates a top down view showing vertical graphene growth on different substrates, in accordance with one embodiment. As an option, the vertical graphene growth may be implemented in the context of any one or more of the embodiments set forth in any previous and/or subsequent Figures and/or description thereof. Of course, however, the vertical graphene growth may be implemented in the context of any desired environment. Further, the aforementioned definitions may equally apply to the description below.

The figure includes a silicon oxide substrate 1002 and a platinum substrate 1004. The vertical graphene structures are shown growing on both substrate materials, with distinct morphological differences visible between growth on the silicon oxide substrate 1002 compared to growth on the platinum substrate 1004.

In some cases, the vertical graphene growth on the silicon oxide substrate 1002 may exhibit a more uniform distribution compared to the growth on the platinum substrate 1004. The silicon oxide substrate 1002 may promote more consistent nucleation and growth of the vertical graphene structures across the surface due to its uniform surface properties and lack of catalytic activity.

The vertical graphene growth on the platinum substrate 1004 may display regions of higher density compared to the growth on the silicon oxide substrate 1002. This variation in density may be attributed to the catalytic properties of platinum during the graphene growth process, potentially leading to localized areas of enhanced growth.

The sensor system exemplifies a resolution to challenges in controlling the growth and distribution of vertical graphene structures on different substrate materials. By demonstrating the ability to grow vertical graphene on both silicon oxide and platinum substrates with distinct morphologies, the sensor system may offer flexibility in device design and fabrication for various sensing applications.

In various embodiments, the growth conditions for the vertical graphene structures may be modified to achieve different distribution patterns or densities on each substrate type. For example, parameters such as growth temperature, gas composition, or plasma power may be adjusted to promote denser or more uniform growth of vertical graphene structures on either the silicon oxide substrate 1002 or platinum substrate 1004.

In various embodiments, the vertical graphene growth patterns observed on the silicon oxide substrate 1002 and platinum substrate 1004 may be leveraged to create hybrid sensing devices. For instance, a single sensor chip may incorporate regions of vertical graphene grown on both substrate types, potentially enabling multi-modal sensing or improved selectivity through the combination of different graphene morphologies.

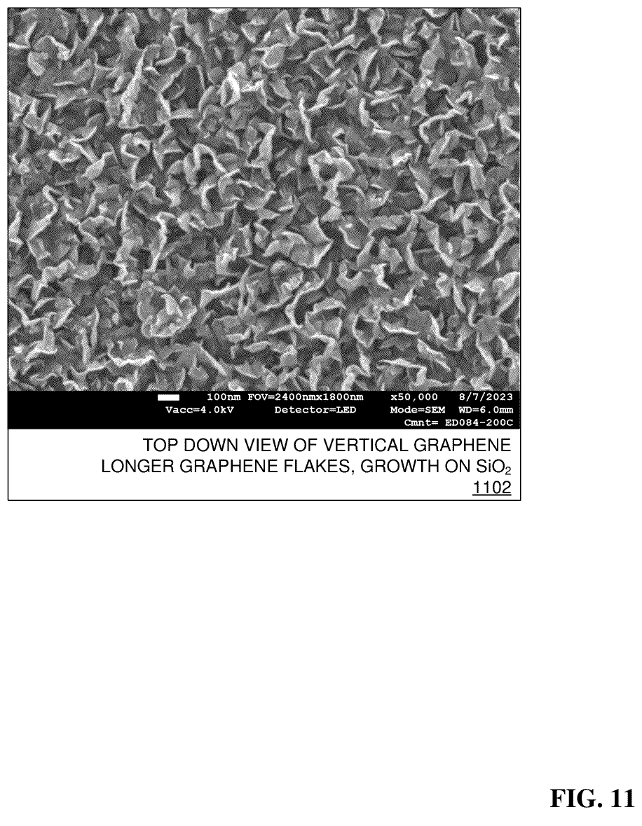

FIG. 11 illustrates a top-down view of a vertical graphene structure grown on a silicon dioxide substrate 1102, in accordance with one embodiment. As an option, the vertical graphene structure grown on the silicon dioxide substrate 1102 may be implemented in the context of any one or more of the embodiments set forth in any previous and/or subsequent Figures and/or description thereof. Of course, however, the vertical graphene structure grown on the silicon dioxide substrate 1102 may be implemented in the context of any desired environment. Further, the aforementioned definitions may equally apply to the description below.

The image shows the morphology of vertically-oriented graphene flakes distributed across the silicon dioxide substrate 1102. The vertical graphene appears as an interconnected network of flake-like structures extending upward from the silicon dioxide substrate 1102. In some cases, the arrangement of the vertical graphene on the silicon dioxide substrate 1102 may create a high surface area structure that can be used for sensing applications.

The vertical graphene exhibits a wrinkled, textured appearance when viewed from above, with the flakes showing varied orientations and connections across the surface of the silicon dioxide substrate 1102. This non-uniform structure may provide multiple binding sites for analytes, potentially enhancing the sensitivity of sensors based on this material. In some cases, the density and distribution of the vertical graphene flakes on the silicon dioxide substrate 1102 may be controlled by adjusting growth parameters such as temperature, gas composition, and plasma conditions during the fabrication process. The ability to tune these characteristics may allow for optimization of the sensor system for specific sensing applications.

The interconnected nature of the vertical graphene network may contribute to improved electrical conductivity across the sensor surface. This enhanced conductivity may lead to more efficient charge transfer and potentially lower detection limits for various analytes in sensing applications.

The interconnected network structure of the vertical graphene on the silicon dioxide substrate 1102 may provide advantages in terms of mechanical stability and resistance to degradation during use. This robust structure may contribute to improved sensor longevity and reliability in field applications compared to more fragile planar graphene sensors.

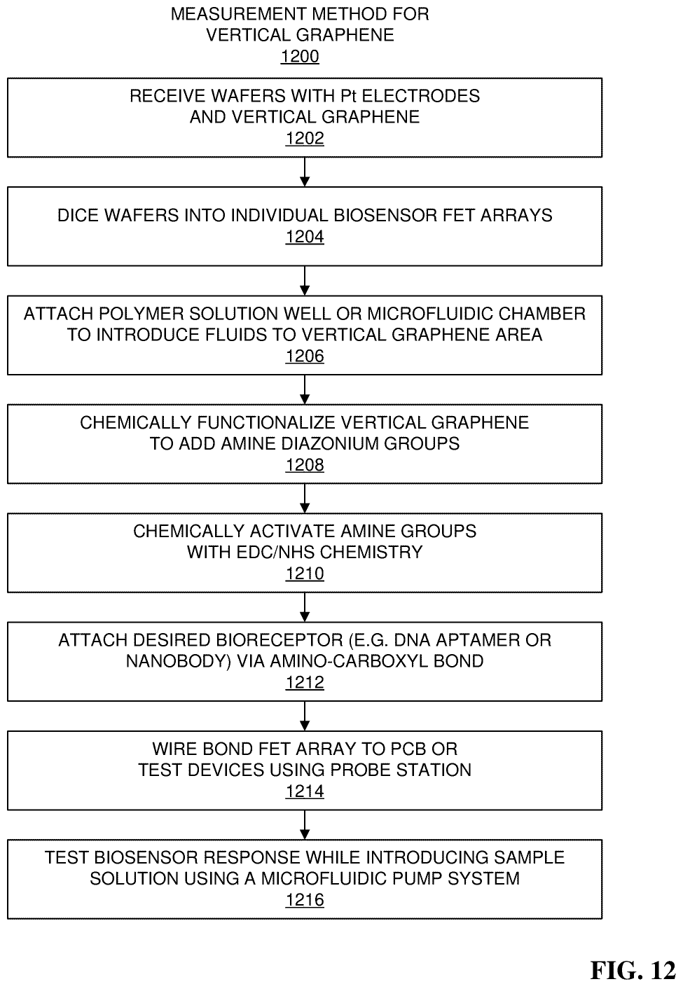

FIG. 12 illustrates a flowchart of a method 1200 for measuring vertical graphene-based biosensors, in accordance with one embodiment. As an option, the method 1200 may be implemented in the context of any one or more of the embodiments set forth in any previous and/or subsequent Figures and/or description thereof. Of course, however, the method 1200 may be implemented in the context of any desired environment. Further, the aforementioned definitions may equally apply to the description below.

The method 1200 begins at a step 1202 with receiving wafers with Pt electrodes and vertical graphene. The method 1200 proceeds to a step 1204, where the wafers are diced into individual biosensor FET arrays.

In operation 1202, the wafers may be prepared using techniques such as photolithography and plasma-enhanced chemical vapor deposition (PECVD) to create the Pt electrodes and vertical graphene structures. The vertical graphene may be grown directly on the Pt electrodes, forming a three-dimensional sensing surface with increased surface area compared to planar graphene sensors. In some cases, the wafers may undergo additional processing steps prior to dicing, such as deposition of passivation layers or annealing treatments to improve the electrical properties of the vertical graphene and Pt electrode interfaces.

In operation 1204, the wafers may be diced using precision cutting techniques such as laser dicing or diamond blade sawing. The dicing process may be optimized to minimize damage to the vertical graphene structures and ensure clean, uniform edges for each individual biosensor FET array. In some cases, the diced biosensor FET arrays may undergo additional cleaning or surface treatment steps to remove any debris or contaminants resulting from the dicing process.

At a step 1206, the method 1200 involves attaching a polymer solution well or microfluidic chamber to introduce fluids to the vertical graphene area. The method 1200 then moves to a step 1208, where the vertical graphene may be chemically functionalized to add amine diazonium groups.

In operation 1206, the polymer solution well or microfluidic chamber may be attached using techniques such as adhesive bonding or thermal bonding. The attachment process may be carefully controlled to ensure a watertight seal around the vertical graphene area while maintaining access for electrical connections to the Pt electrodes. In some cases, the polymer solution well or microfluidic chamber may be fabricated using materials compatible with the target analytes and measurement conditions, such as polydimethylsiloxane (PDMS) or other biocompatible polymers.

In operation 1208, the chemical functionalization of the vertical graphene may involve exposure to diazonium salt solutions under controlled conditions. The amine diazonium groups may form covalent bonds with the graphene surface, providing reactive sites for subsequent attachment of bioreceptors. In some cases, the functionalization process may be optimized to achieve a uniform distribution of amine groups across the vertical graphene surface while maintaining the electrical properties of the graphene structures.

The method 1200 continues at a step 1210 with chemically activating the amine groups with EDC/NHS chemistry. At a step 1212, the method 1200 involves attaching a desired bioreceptor via amino-carboxyl bond, where the bioreceptor may be a DNA aptamer or nanobody.

In operation 1210, the EDC/NHS (1-Ethyl-3-(3-dimethylaminopropyl) carbodiimide/N-hydroxysuccinimide) chemistry may be used to activate the amine groups on the vertical graphene surface. This activation step may prepare the surface for efficient coupling with the carboxyl groups present on the bioreceptors. In some cases, the EDC/NHS activation may be performed under carefully controlled pH and temperature conditions to maximize the efficiency of the reaction while minimizing potential damage to the vertical graphene structures.

In operation 1212, the attachment of bioreceptors may involve incubating the activated vertical graphene surface with a solution containing the desired DNA aptamers or nanobodies. The amino-carboxyl bond formation may create a stable linkage between the bioreceptors and the vertical graphene surface. In some cases, the bioreceptor attachment process may be followed by washing steps to remove any unbound molecules and blocking steps to prevent non-specific binding during subsequent sensing experiments.

The method 1200 then proceeds to a step 1214, where the FET array may be wire bonded to a PCB or test devices using a probe station. Finally, at a step 1216, the method 1200 involves testing the biosensor response while introducing sample solution using a microfluidic pump system.

In operation 1214, the wire bonding process may create electrical connections between the Pt electrodes of the biosensor FET array and the corresponding pads on a PCB or test device. The wire bonding may be performed using techniques such as thermosonic bonding or ultrasonic bonding to ensure reliable electrical connections. In some cases, the wire bonding process may be followed by encapsulation or protective coating steps to safeguard the delicate wire bonds from mechanical stress or environmental factors.

In operation 1216, the biosensor response testing may involve introducing sample solutions containing target analytes to the functionalized vertical graphene surface using a microfluidic pump system. The electrical response of the FET array may be measured and recorded as the analytes interact with the bioreceptors on the vertical graphene surface. In some cases, the testing process may include multiple cycles of sample introduction and washing steps to evaluate the sensitivity, specificity, and reproducibility of the biosensor response.

The method 1200 exemplifies a resolution to challenges in fabricating and testing vertical graphene-based biosensors. By combining precise fabrication techniques with controlled surface functionalization and electrical integration, the method 1200 may enable the production of highly sensitive and specific biosensing devices.

The integration of microfluidic systems and automated testing procedures may allow for reproducible and efficient evaluation of biosensor performance, potentially accelerating the development and optimization of vertical graphene-based sensing platforms for various applications.

In various embodiments, the method 1200 may be modified to incorporate additional processing steps or alternative techniques for specific biosensing applications. For example, the vertical graphene functionalization process may be adapted to include different chemical groups or bioreceptors tailored for detecting specific biomarkers or environmental contaminants.

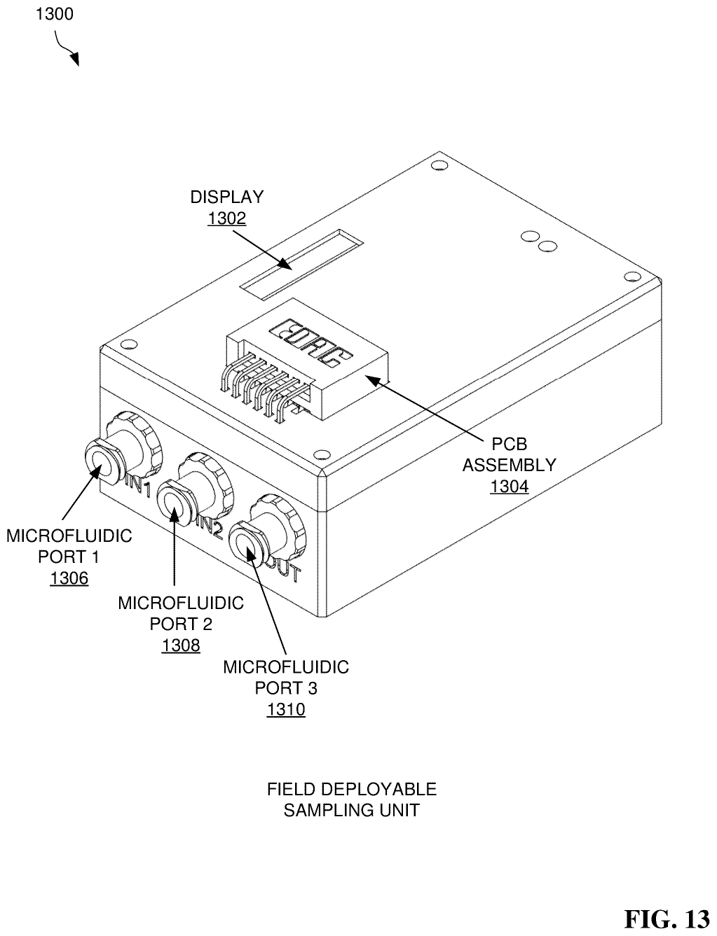

FIG. 13 illustrates an isometric view of a field deployable sampling unit 1300, in accordance with one embodiment. As an option, the field deployable sampling unit 1300 may be implemented in the context of any one or more of the embodiments set forth in any previous and/or subsequent Figures and/or description thereof. Of course, however, the field deployable sampling unit 1300 may be implemented in the context of any desired environment. Further, the aforementioned definitions may equally apply to the description below.

The field deployable sampling unit 1300 includes a display screen 1302 positioned on the top surface of the unit for providing visual information to a user. A PCB assembly 1304 may be contained within the housing of the unit. The field deployable sampling unit 1300 includes three microfluidic ports-a microfluidic port 1306, a microfluidic port 1308, and a microfluidic port 1310—arranged in a row along one side of the housing.

In some cases, the display screen 1302 may be a touchscreen interface, allowing users to input commands and adjust settings directly on the field deployable sampling unit 1300. Additionally, the PCB assembly 1304 may contain the electronic components necessary for controlling the sampling and measurement processes. In some cases, the PCB assembly 1304 may include microcontrollers, analog-to-digital converters, and communication modules for data processing and transmission.

The microfluidic ports 1306, 1308, 1310 may enable fluid samples to be introduced into and removed from the sampling unit 1300. In some cases, these ports may be connected to internal microfluidic channels that guide the sample to the sensor system for analysis.

The housing of the field deployable sampling unit 1300 may have a compact rectangular form factor suitable for portable field use. In some cases, the housing may be made of durable, lightweight materials to withstand environmental conditions encountered during field deployments.

A microperistaltic pump (not shown) may be included within the field deployable sampling unit 1300 to control fluid flow through the system. In some cases, the microperistaltic pump may be connected to the microfluidic ports 1306, 1308, 1310 via internal tubing to enable precise control over sample introduction and removal.

The field deployable sampling unit 1300 may also include a flow sensor to monitor and regulate fluid flow rates during sampling and measurement processes. In some cases, the flow sensor may provide feedback to the PCB assembly 1304 to ensure consistent and reproducible sample handling across different measurements.

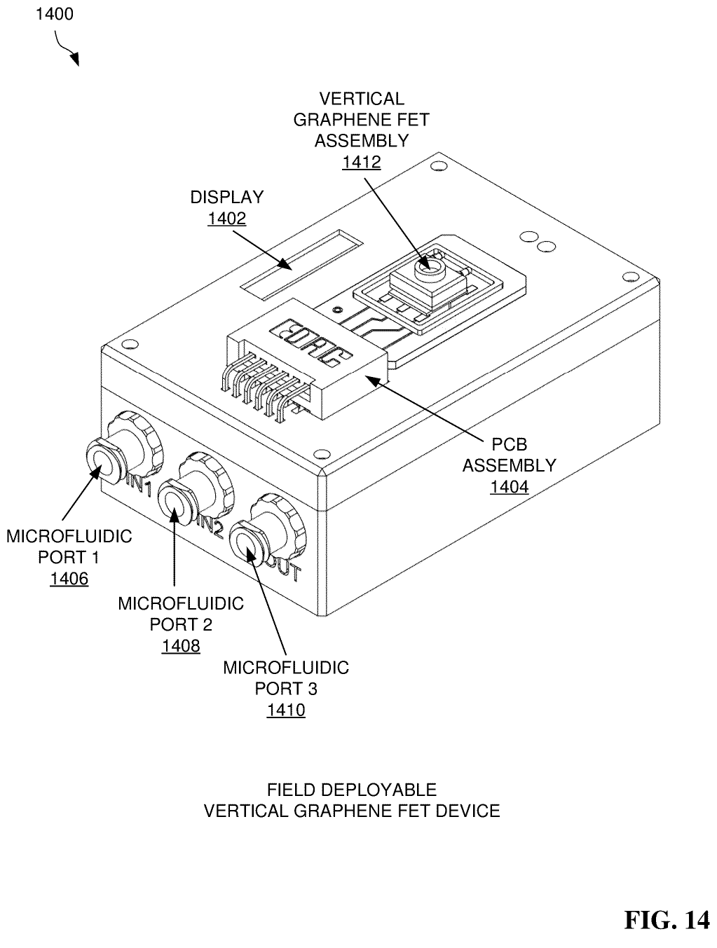

FIG. 14 illustrates an isometric view of a field deployable vertical graphene FET device 1400, in accordance with one embodiment. As an option, the field deployable vertical graphene FET device 1400 may be implemented in the context of any one or more of the embodiments set forth in any previous and/or subsequent Figures and/or description thereof. Of course, however, the field deployable vertical graphene FET device 1400 may be implemented in the context of any desired environment. Further, the aforementioned definitions may equally apply to the description below.

The field deployable vertical graphene FET device 1400 includes a display 1402 positioned on the upper surface for showing measurement data and system status information. A PCB assembly 1404 may be contained within the device housing and may provide electronic control and measurement functionality. The device 1400 includes three microfluidic ports—a microfluidic port 1406, a microfluidic port 1408, and a microfluidic port 1410—arranged along one side of the housing to enable fluid sample introduction and flow control. A vertical graphene FET assembly 1412 may be integrated within the device 1400 and may interface with both the PCB assembly 1404 for electrical measurements and the microfluidic ports for sample exposure.

In some cases, the display 1402 may be an LED display configured to show diagnostic information related to the sensor system operation. The display 1402 may provide real-time feedback on parameters such as flow rates, applied voltages, and measurement results to facilitate user interaction with the device 1400.

The PCB assembly 1404 may contain the electronic components necessary for controlling the sampling and measurement processes. In some cases, the PCB assembly 1404 may include microcontrollers, analog-to-digital converters, and communication modules for data processing and transmission.

The microfluidic ports 1406, 1408, 1410 may enable fluid samples to be introduced into and removed from the device 1400. In some cases, these ports may be connected to internal microfluidic channels that guide the sample to the vertical graphene FET assembly 1412 for analysis.

The vertical graphene FET assembly 1412 may incorporate the vertical graphene structures described in previous figures, such as the vertical graphene 306 shown in FIG. 3. In some cases, the vertical graphene FET assembly 1412 may be configured with multiple sensing elements to enable simultaneous detection of different analytes.

The field deployable vertical graphene FET device 1400 may provide an integrated portable platform that combines vertical graphene sensing capabilities with automated fluid handling and measurement functions in a compact form factor suitable for field deployment. The device 1400 may enable on-site analysis of biological and chemical samples without the need for complex laboratory equipment.

In some cases, the sensor system within the field deployable vertical graphene FET device 1400 may be configured to sweep the gate electrode voltage from −0.1V to 0.9V during measurements. This voltage sweep may allow for the characterization of the vertical graphene FET response across a range of operating conditions, potentially enhancing the sensitivity and selectivity of the sensor.

The sensor system may apply a bias voltage of 50-300 mV to the graphene electrodes during measurements. In some cases, this bias voltage may be optimized for specific analyte detection or to minimize interference from other electrochemical processes in the sample solution.

It is to be appreciated that FIG. 14 illustrates a field deployable vertical graphene FET device 1400 that represents a fully integrated solution, building upon the portable sampling unit shown in FIG. 13. While FIG. 13 depicts a field deployable sampling unit 1300 that primarily focuses on sample handling and basic measurement capabilities, FIG. 14 incorporates a vertical graphene FET assembly 1412 directly into the device. This integration combines the advanced sensing capabilities of vertical graphene field-effect transistors with the portability and automated fluid handling features of the sampling unit. By incorporating the vertical graphene FET assembly 1412, the device in FIG. 14 offers a complete, self-contained biosensing platform capable of on-site sample analysis, potentially enabling more rapid and sensitive detection of analytes in field settings compared to the sampling-only unit shown in FIG. 13.

The sensor system of FIG. 14 exemplifies a resolution to challenges in creating portable, field-deployable biosensing devices with high sensitivity and specificity. By integrating the vertical graphene-based sensor array with automated fluid handling and measurement capabilities, the field deployable vertical graphene FET device 1400 may enable rapid, on-site analysis of complex biological and chemical samples. Additionally, the compact and integrated design of the field deployable vertical graphene FET device 1400 may allow for easy transportation and use in various environments, potentially expanding the range of applications for graphene-based biosensors beyond traditional laboratory settings.

In various embodiments, the microfluidic system of the field deployable vertical graphene FET device 1400 may be expanded to include on-board sample preparation capabilities. This may involve the integration of mixing chambers, filtration units, or reagent storage compartments to enable more complex analytical protocols in field settings.

In various embodiments, the field deployable vertical graphene FET device 1400 may be equipped with wireless communication capabilities to enable real-time data transmission and remote monitoring. This feature may allow for the rapid sharing of analytical results with centralized laboratories or decision-makers, potentially improving response times in environmental monitoring or healthcare applications.

By way of a use-case scenario, and in various embodiments, a field researcher investigating water quality in remote areas deploys the field deployable vertical graphene FET device 1400 to conduct on-site analysis of potentially contaminated water sources. The researcher collects water samples from various locations and uses the microfluidic ports 1406, 1408, and 1410 to introduce the samples into the device. The vertical graphene FET assembly 1412 within the device, functionalized with specific bioreceptors, detects the presence and concentration of target contaminants such as heavy metals or bacterial toxins. The PCB assembly 1404 controls the measurement process, sweeping the gate electrode voltage (such as from −0.1V to 0.9V) and applying a bias voltage (such as 50-300 mV) to the graphene electrodes. Real-time results are displayed on the screen 1402, allowing the researcher to immediately identify contaminated water sources. The device's wireless communication capabilities enable the researcher to transmit the analytical data to a central laboratory for further analysis and rapid response planning. This portable, automated biosensing system allows for quick, accurate, and on-site water quality assessment in areas where traditional laboratory analysis would be impractical or time-consuming, potentially improving public health outcomes in remote or underserved regions.

By way of a second use-case scenario, a medical technician in a remote clinic deploys the field deployable vertical graphene FET device 1400 to conduct rapid, on-site testing for a specific biomarker associated with an infectious disease outbreak. The technician begins by collecting a small blood sample from a patient and diluting it with a buffer solution. Using one of the microfluidic ports (1406, 1408, or 1410), the technician introduces the prepared sample into the device 1400. The integrated microfluidic system within the device automatically guides the sample to the vertical graphene FET assembly 1412. The vertical graphene FET assembly 1412, which has been pre-functionalized with antibodies specific to the target biomarker, interacts with the sample. As the sample flows over the vertical graphene structures, any present biomarkers bind to the antibodies on the graphene surface. The PCB assembly 1404 controls the measurement process, applying a series of voltage sweeps to the vertical graphene FET while monitoring the electrical response. The binding of biomarkers to the antibodies causes changes in the electrical properties of the graphene, which are detected and measured by the device. Real-time data processing algorithms within the PCB assembly 1404 analyze the changes in electrical signals, correlating them with biomarker concentration levels. The results are quickly displayed on the display 1402, showing the presence and approximate concentration of the target biomarker. In this case, the device 1400 detects an elevated level of the biomarker, indicating a positive result for the infectious disease. The technician can immediately read and interpret the results on the display 1402, allowing for rapid decision-making regarding patient care and isolation procedures. The entire testing process, from sample introduction to result display, takes only a few minutes, significantly faster than traditional laboratory-based testing methods. The field deployable nature of the device 1400 allows for immediate testing and results, crucial in managing potential outbreaks in remote or resource-limited settings. Additionally, after the test, the device 1400 can be easily cleaned and prepared for the next sample, allowing for multiple tests to be conducted in quick succession. The portability and rapid testing capabilities of the field deployable vertical graphene FET device 1400 enable efficient on-site disease screening, potentially improving response times and patient outcomes in outbreak scenarios.

The present disclosure addresses significant challenges in the field of portable biosensing that have limited the effectiveness of existing technologies. Prior art solutions have struggled to achieve the sensitivity and specificity of laboratory-based methods while maintaining a compact form factor suitable for field deployment. Conventional portable biosensors often lack sophisticated control over sample exposure and measurement conditions, leading to inconsistent results. Additionally, many current systems face issues related to sensor stability, cross-reactivity with interfering substances, and the ability to detect multiple analytes simultaneously. These limitations have hindered the widespread adoption of portable biosensors in critical applications such as environmental monitoring, healthcare diagnostics, and food safety testing.

The disclosed automated portable biosensor system overcomes these deficiencies through a novel approach that uses three-dimensional graphene-based sensing elements. By utilizing vertical graphene field effect transistor (FET) arrays, the system achieves high sensitivity and specificity in a compact, field-deployable format. The vertical graphene structure provides increased surface area for analyte binding, while the automated sample handling and measurement processes ensure consistent and reliable results without the need for specialized operators. This innovative approach not only enables rapid, on-site analysis of liquid samples but also offers the flexibility to detect a wide range of analytes through its modular design, effectively addressing the longstanding issues of portability, sensitivity, and versatility that have plagued prior art biosensing systems.

Moving on to FIGS. 15-1-15-14C, batteries typically include a plurality of electrochemical cells that can be used to power a wide variety of devices including, for example, mobile phones, laptops, and electric vehicles (EVs), factories, and buildings. When batteries are exposed to harsh environmental conditions or become damaged, toxic chemicals and vapors within the electrochemical cells may leak from the battery's casing and pose serious health and safety risks. When released from a battery, these toxic chemicals and vapors can cause respiratory problems, allergic reactions, and may even explode. The chemicals typically used in the cells of Lithium-ion batteries may be particularly dangerous due to their high reactivities and susceptibility to explosion when inadvertently released from the battery casing. As such, there is a need to quickly and accurately determine whether a particular battery or battery pack is leaking such toxic chemicals or vapors. Moreover, when the presence of one or more analytes (or other toxic chemicals or vapors) is detected, it may be desirable to determine the concentration of such analytes. It may also be desirable to predict battery failure and/or to determine the operational integrity of such batteries.

Various aspects of the subject matter disclosed herein relate to detecting a presence of one or more analytes in an environment. In accordance with various implementations of the subject matter disclosed herein, a sensing device may include a plurality of carbon-based sensors configured to the presence of a variety of different analytes. In some implementations, at least some of the carbon-based sensors may include different types of three-dimensional (3D) graphene-based sensing materials configured to react with different analytes or different groups of analytes. In some aspects, the sensing materials of different sensors may be functionalized with different materials, for example, to increase the sensitivity of each sensor to one or more corresponding analytes.

In some implementations, changes in the impedances of the sensors may be used to determine a presence of one or more analytes in a vicinity of the sensing device. In other implementations, changes in current flow through the sensors may be used to determine the presence of the one or more analytes in the vicinity of the sensing device. In some other implementations, frequency responses of the sensors may be used to determine the presence of the one or more analytes in the vicinity of the sensing device. In some aspects, the frequency responses of the sensors may be compared with one or more reference frequency responses corresponding to the one or more analytes to identify which analytes are present in the environment. In this way, the sensor systems disclosed herein can accurately detect the presence of a variety of different analytes in a given environment.

In one implementation, a first sensor may be configured to detect the presence of a relatively large number of different analytes, and one or more second sensors may be configured to confirm the presence of one or more analytes detected by the first sensor. Specifically, the first sensor may be configured to react with each analyte of a first group of analytes, and the one or more second sensors may be configured to react with corresponding second groups of analytes that are unique subsets of the first group of analytes. In some instances, the first sensor may be exposed to the surrounding environment for a relatively short period of time to provide an initial coarse indication of whether the analytes of the first group of analytes are present, and each of the second sensors may be exposed to the surrounding environment for a relatively long period of time to provide a fine indication of whether any of the analytes of the corresponding second group of analytes are present. For example, while the first sensor may be able to detect a greater number of analytes than any of the second sensors, configuring each of the second sensors to detect only one or two different analytes may increase the sensitivity of the second sensors to their respective “target” analytes, thereby increasing the accuracy with which the sensing device is able to detect the presence of various analytes. As such, when indications provided by the second sensors are used to confirm indications provided by the first sensor, the number of false positive indications decreases, which in turn increases overall accuracy of the sensing device.

Particular implementations of the subject matter described in this disclosure can be implemented to realize one or more of the following potential advantages. In some implementations, the sensing devices disclosed herein can not only detect the presence of a variety of analytes and other harmful chemicals and gases, but can also reduce the occurrence of false positives. Specifically, by using a first sensor to quickly detect a presence of one or more analytes of a group of analytes and using one or more second sensors to confirm the presence of analytes detected by the first sensor, aspects of the present disclosure can reduce the number of false positives indicated by the sensing device. This is in contrast to conventional analyte sensors that may not only be insensitive to differences between different analytes of a group of analytes and/or that do not employ a multi-tiered analyte detection system.

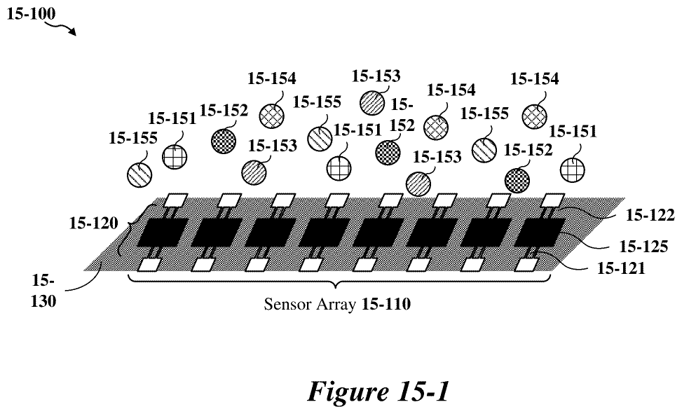

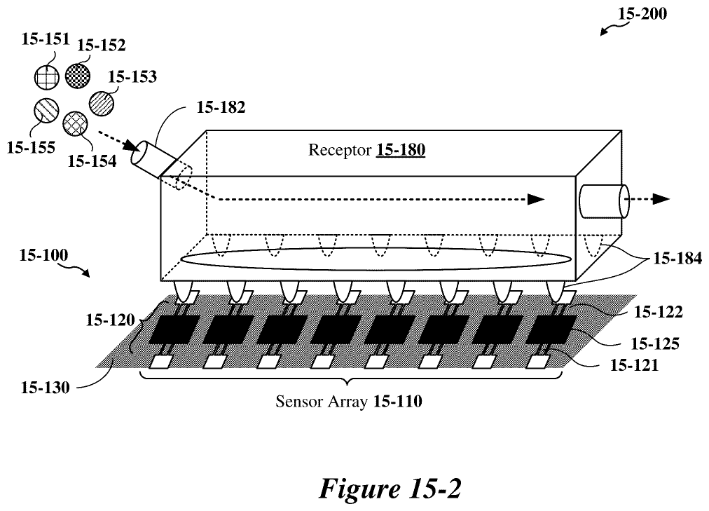

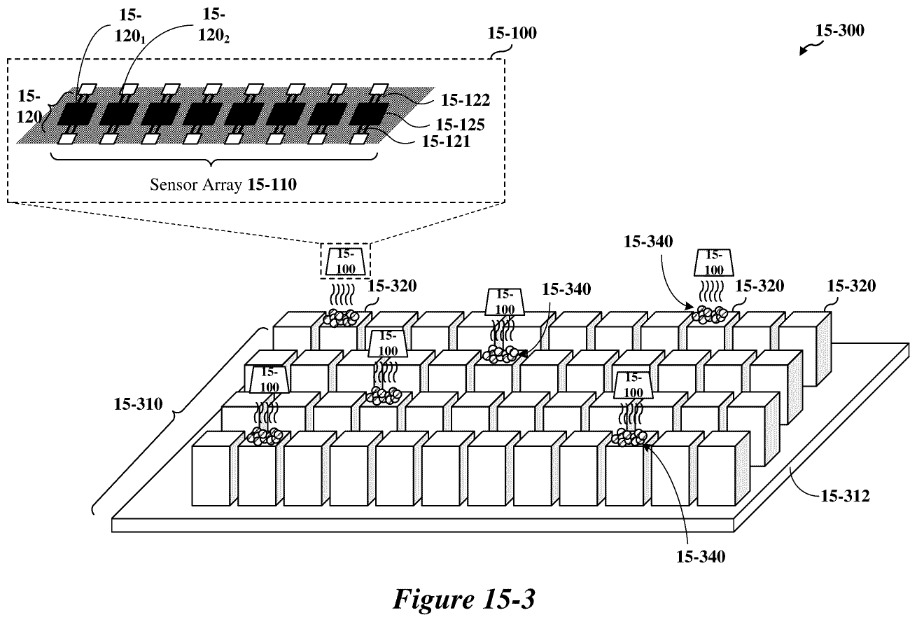

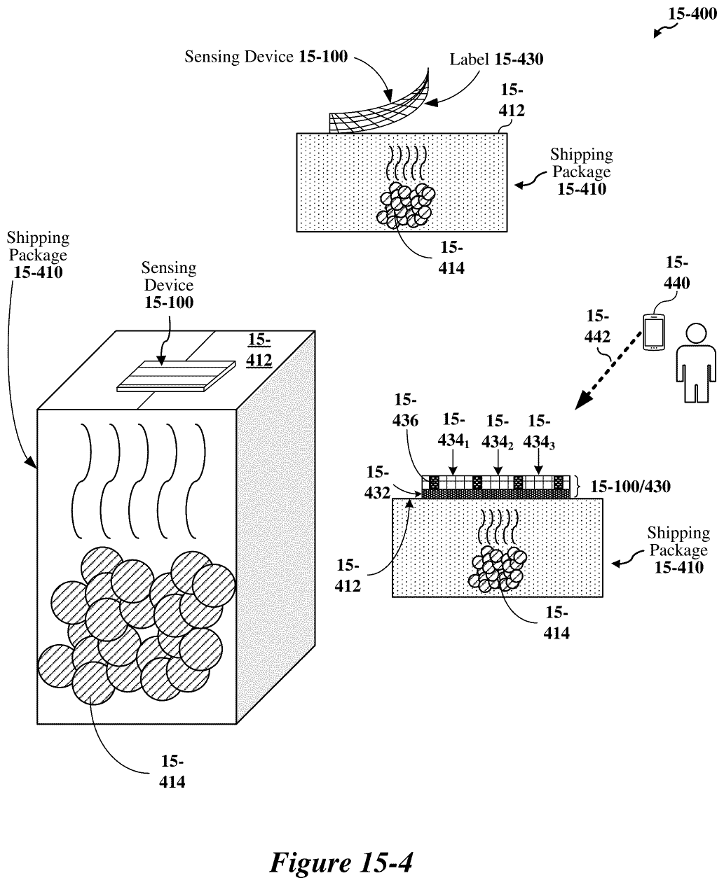

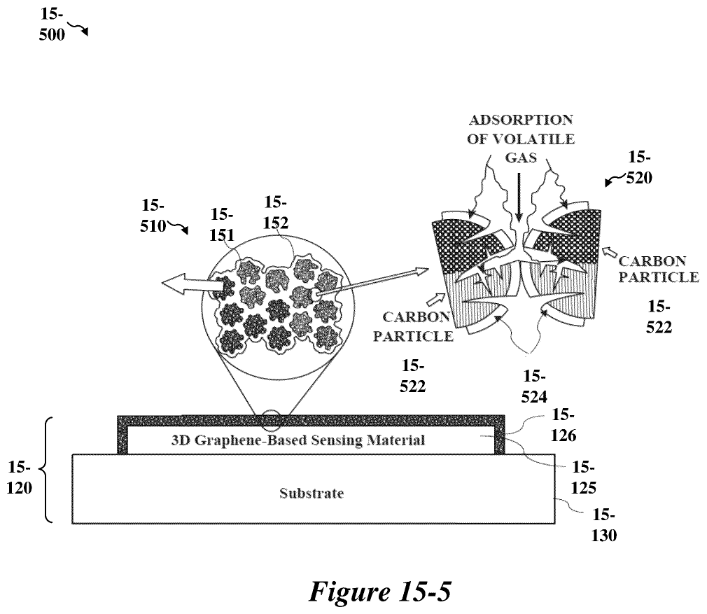

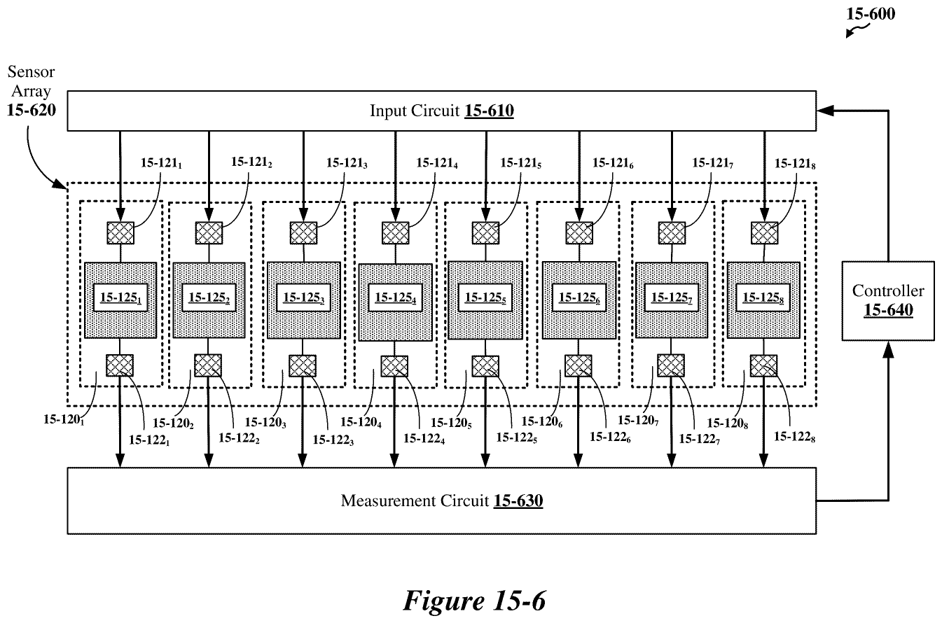

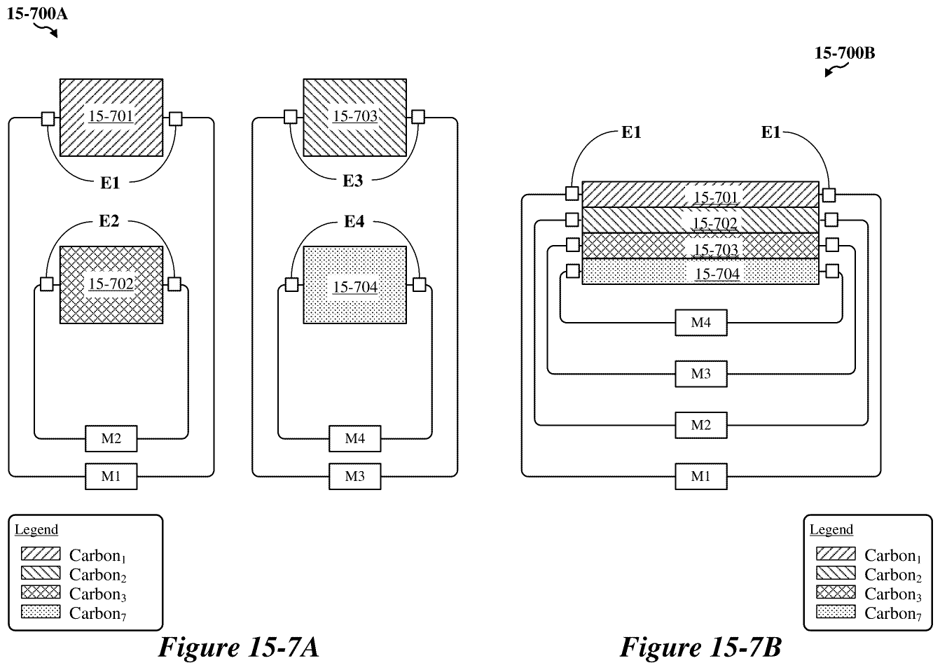

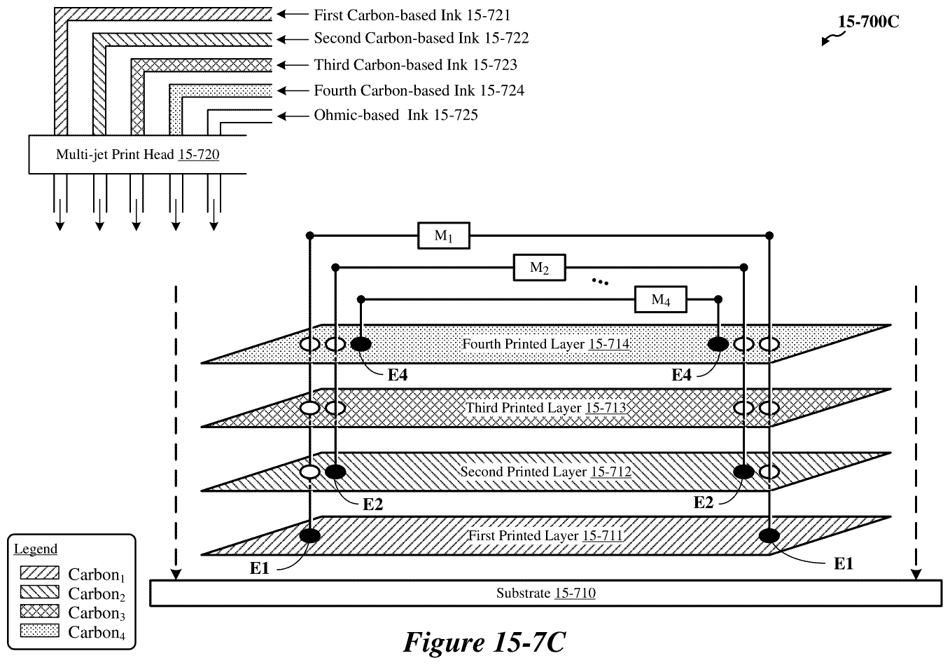

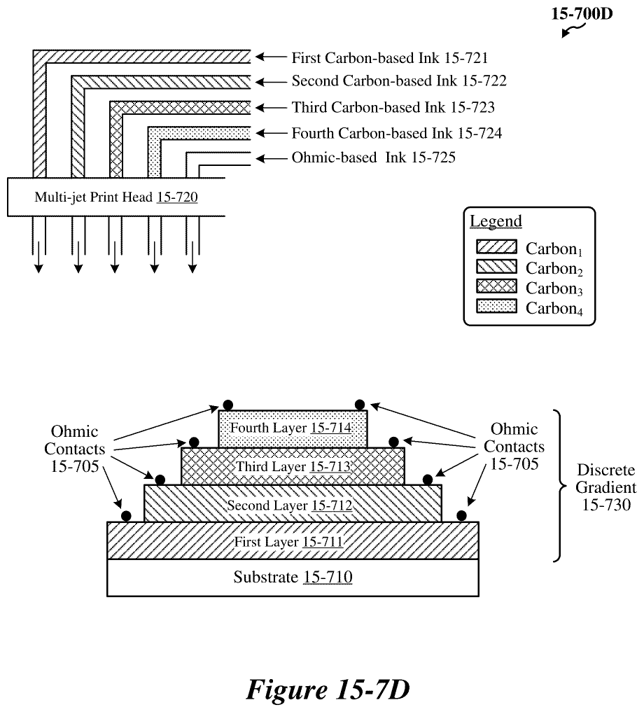

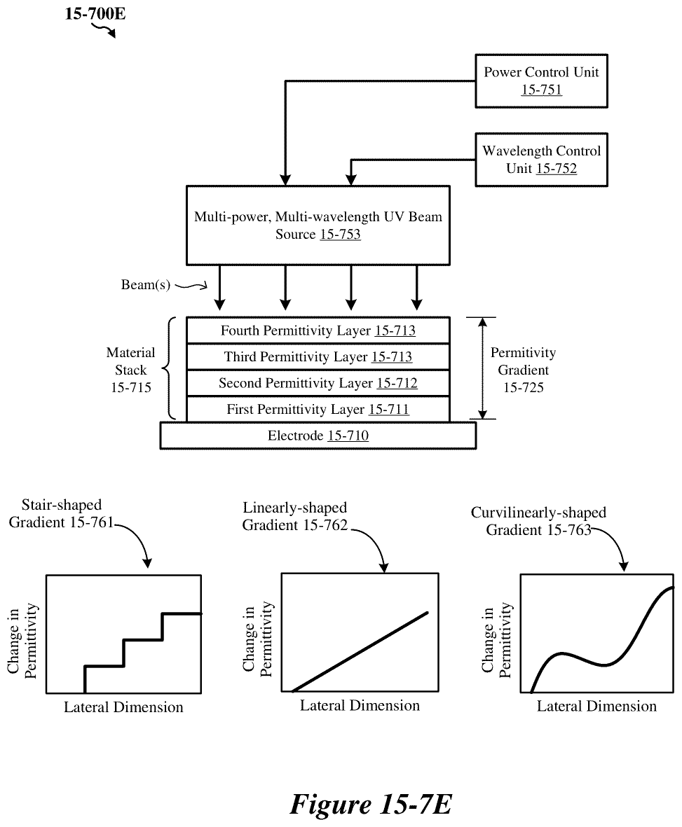

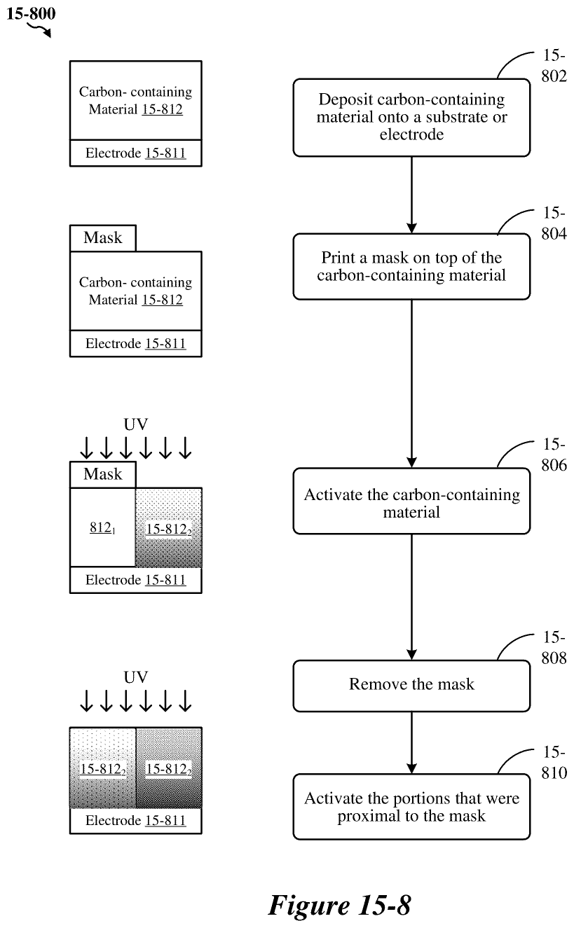

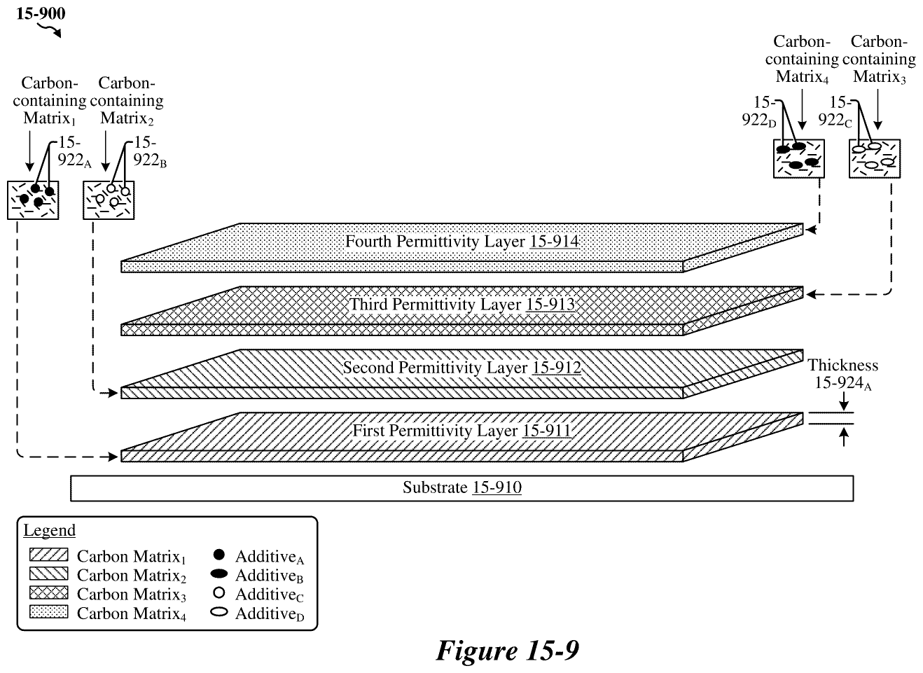

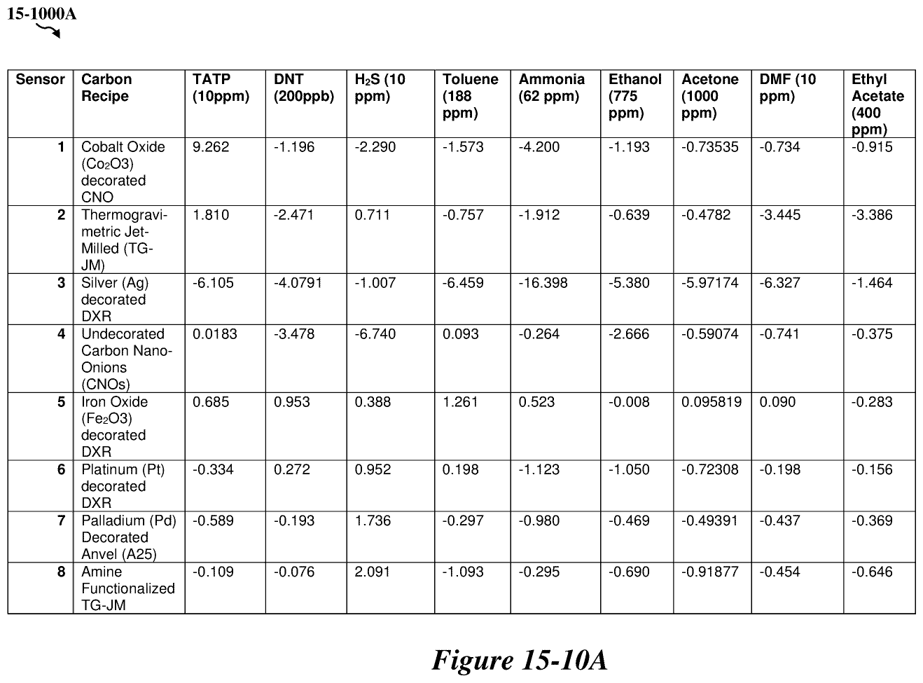

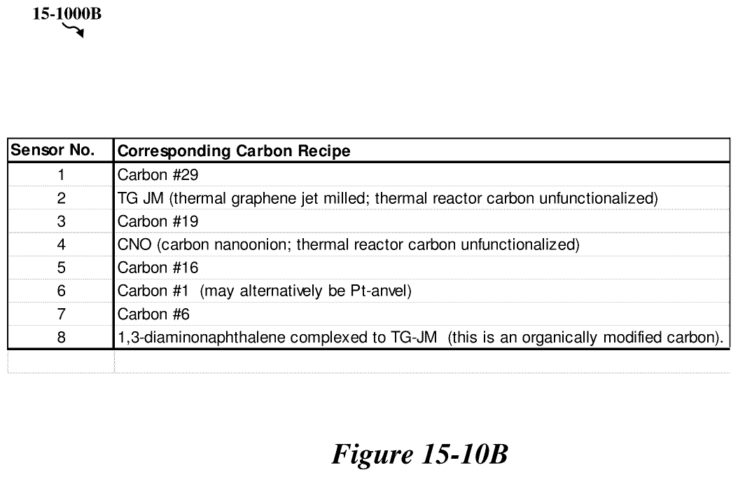

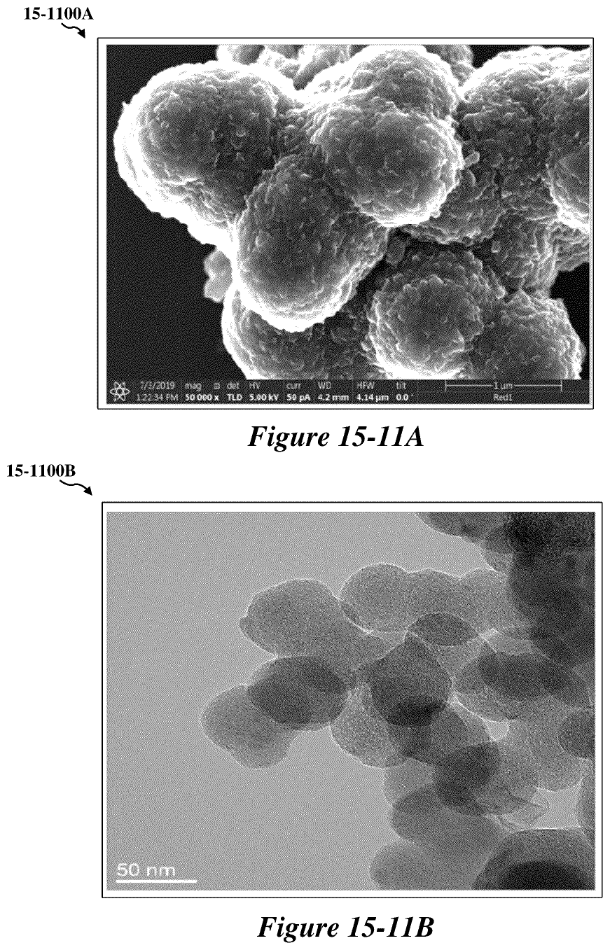

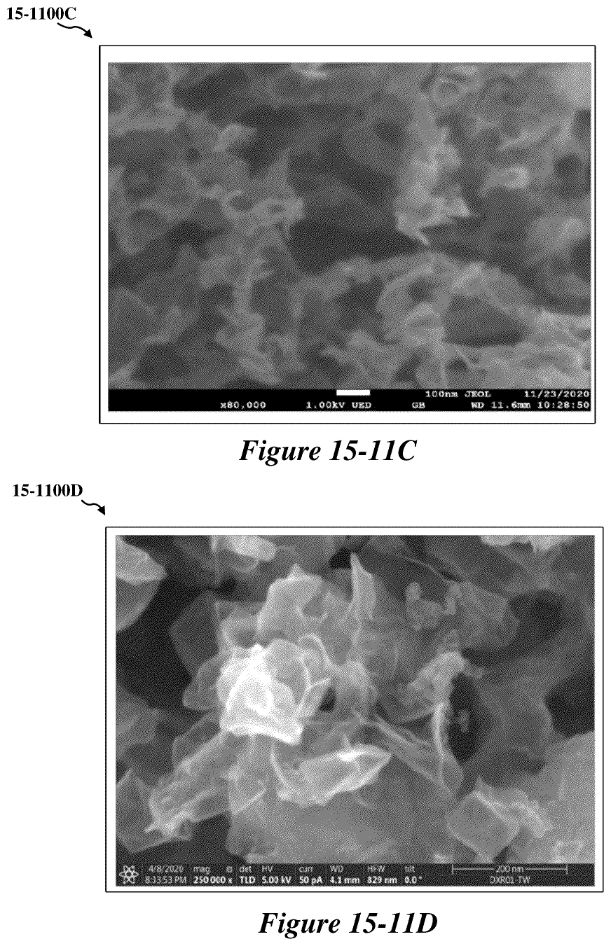

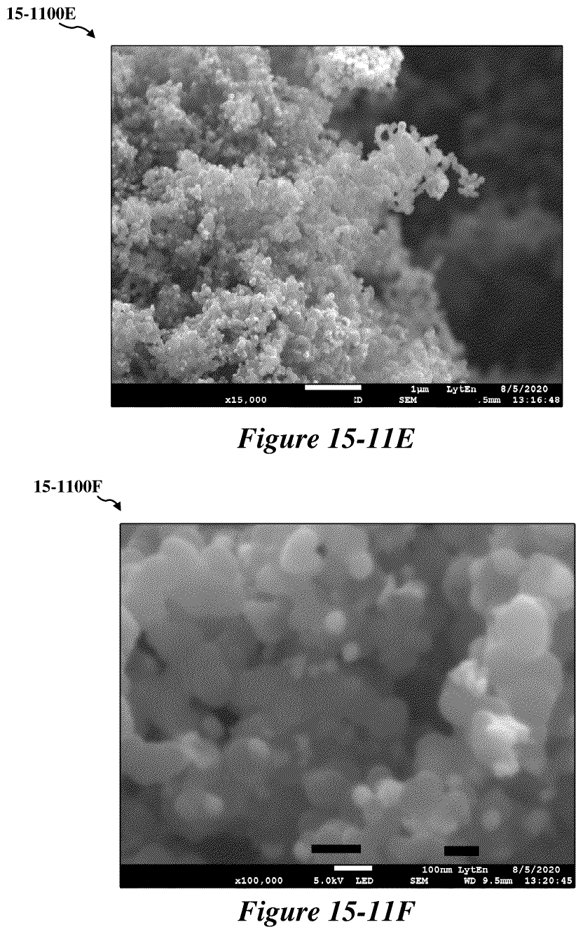

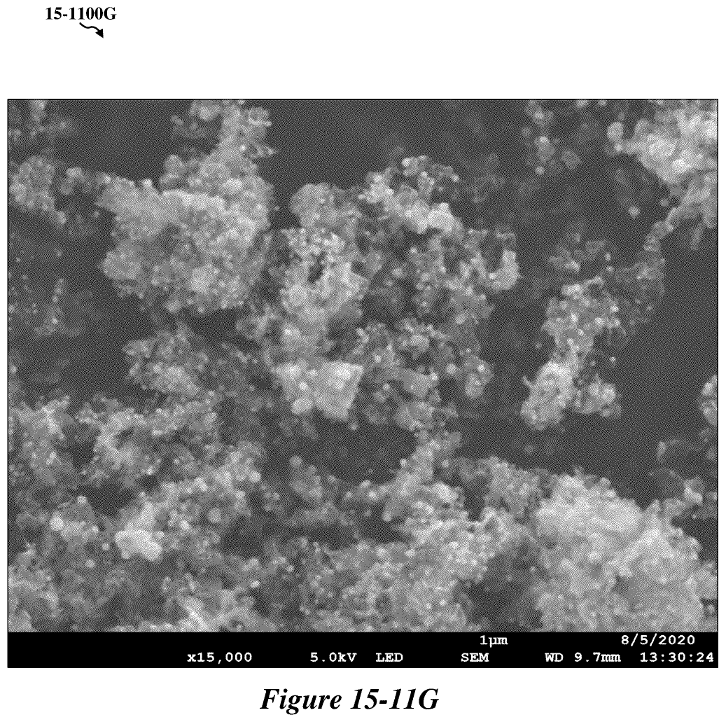

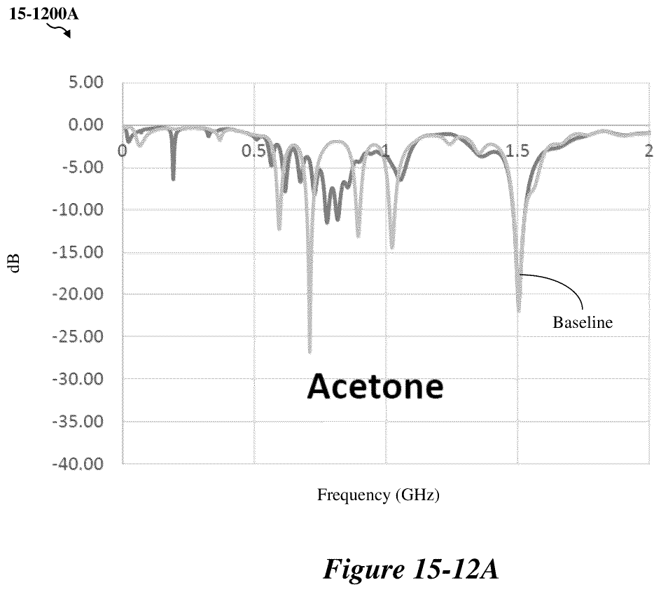

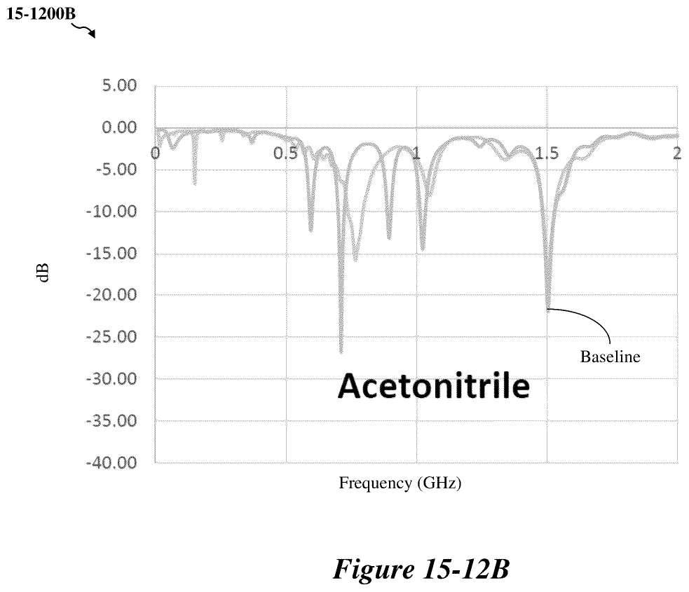

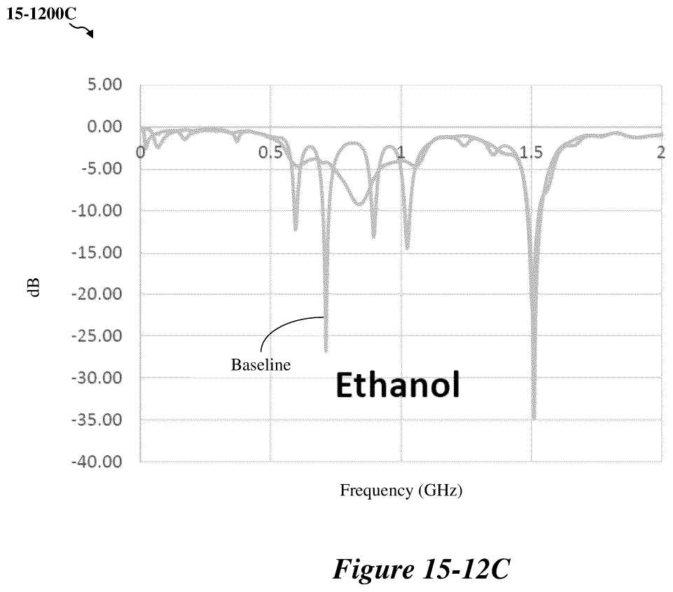

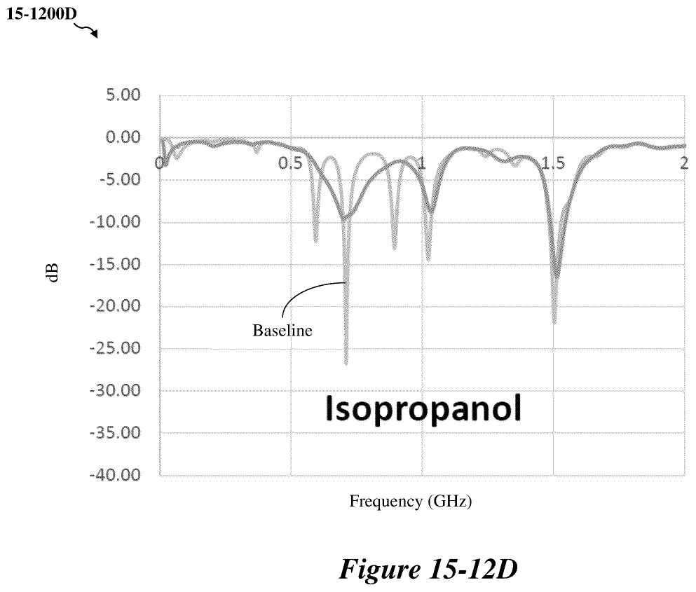

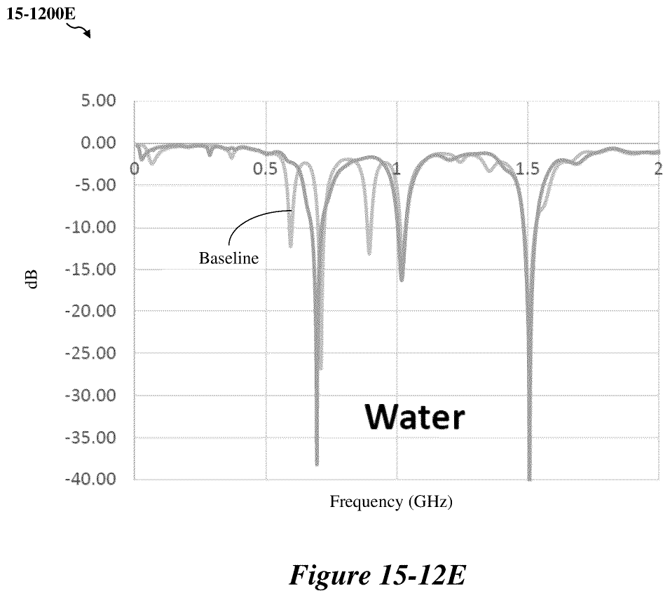

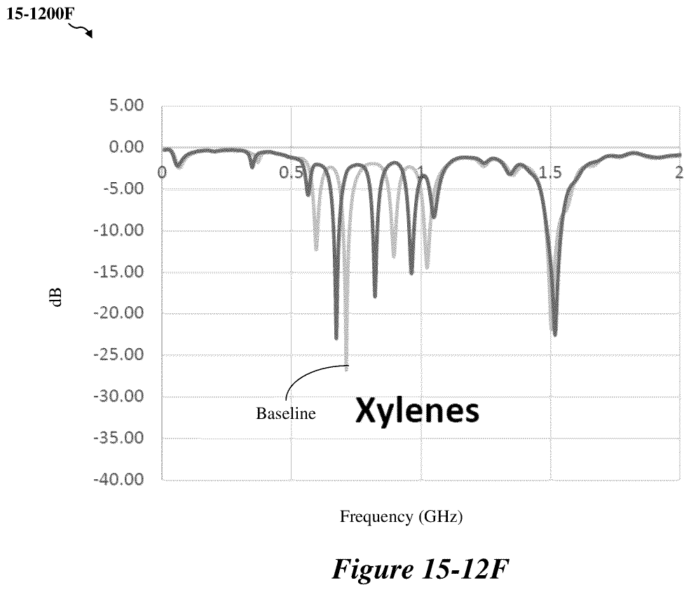

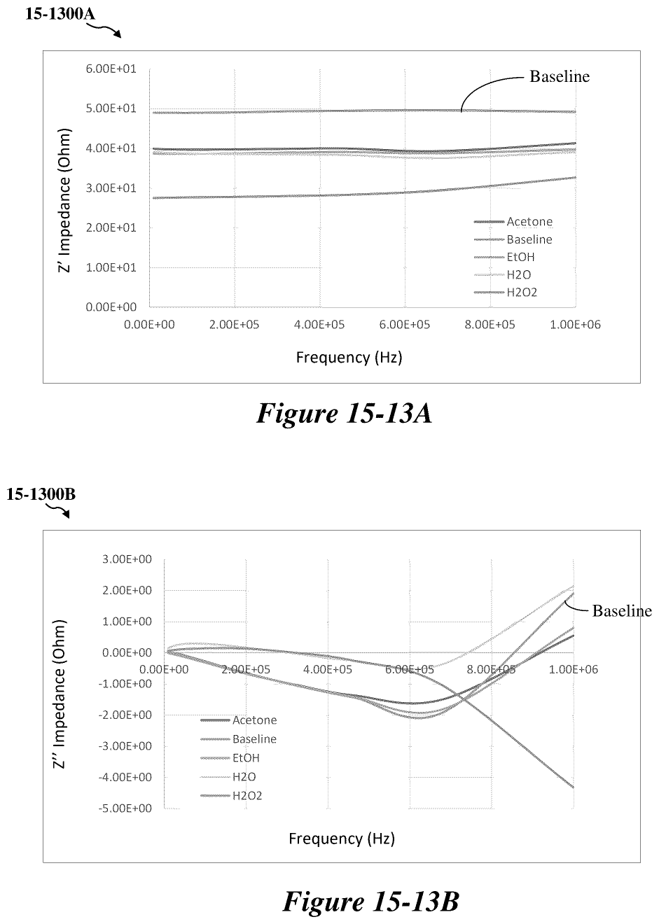

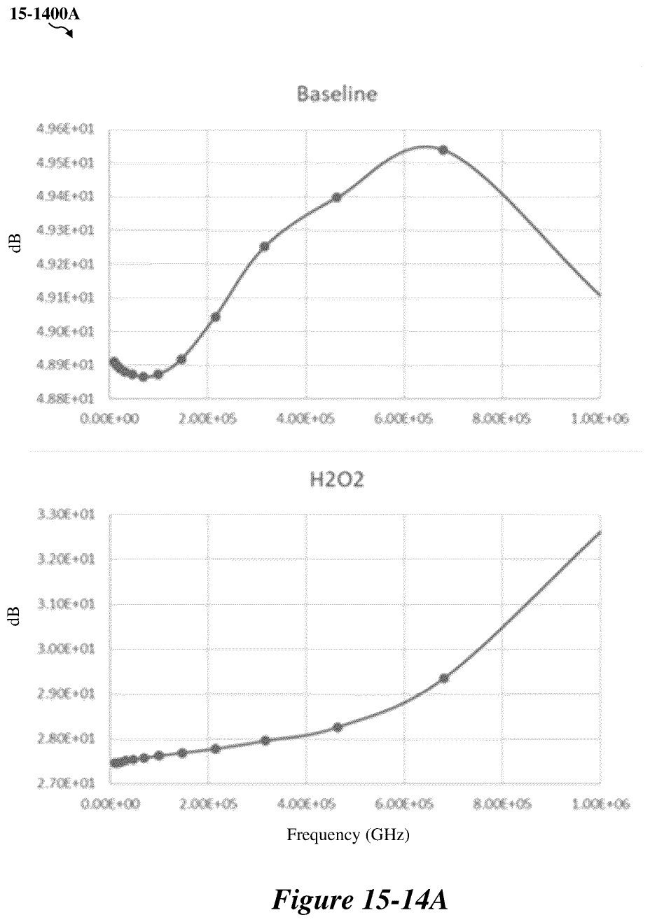

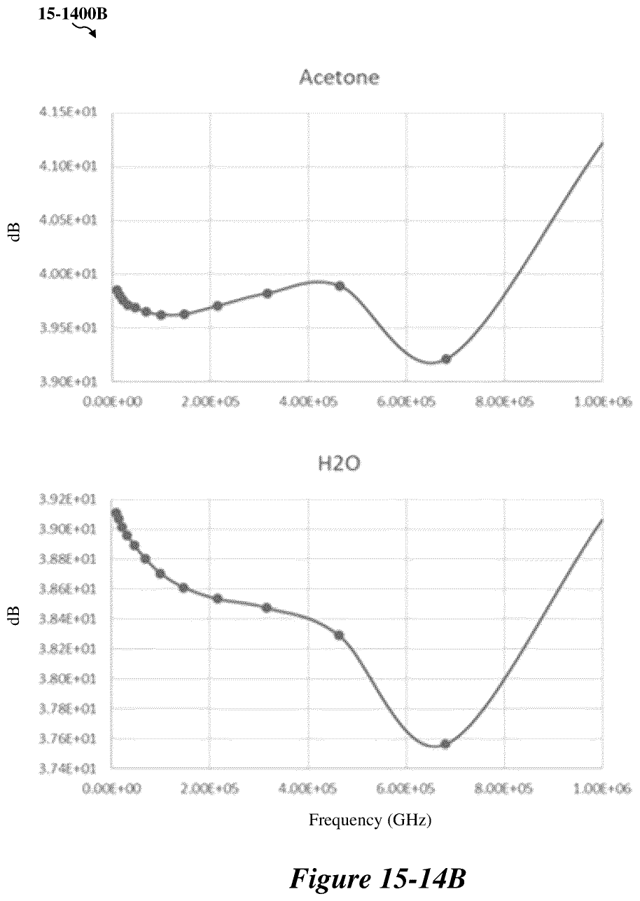

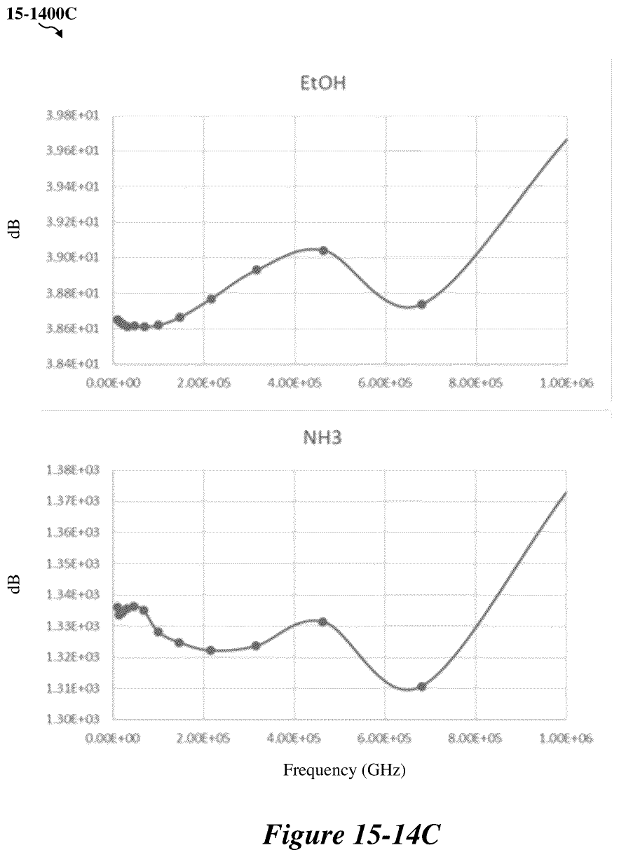

FIG. 15-1 shows an example sensing device 15-100 configured to detect analytes, according to some implementations. The sensing device 15-100 may include an array 15-110 of carbon-based sensors 15-120 disposed on a substrate 15-130. In some aspects, each of the carbon-based sensors 15-120 may include a carbon-based sensing material 15-125 disposed between a corresponding pair of electrodes 15-121-122, for example, as depicted in FIG. 15-1. In other aspects, the carbon-based sensors 15-120 may be coupled to only one electrode. The carbon-based sensors 15-120, as well as their respective carbon-based sensing materials 15-125, may be formed from any suitable materials that react to, or that can be configured to react to, a variety of different analytes. Reactions between the carbon-based sensors 15-120 and various analytes may be used to detect a presence of a particular analyte or a particular group of analytes. For example, the reactions may cause changes in current flow through one or more carbon-based sensors 15-120, may cause changes in the impedances or reactance of one or more carbon-based sensors 15-120, may produce unique or different frequency responses in one or more carbon-based sensors 15-120, or any combination thereof.

In the example of FIG. 15-1, a plurality of different analytes 15-151-15-155 are in the presence of the sensing device 15-100. Although only five analytes 15-151-15-155 are shown in FIG. 15-1, the sensing device 15-100 can detect a greater number of different analytes. In some aspects, the analytes 15-151-15-155 can include any vapor phase and/or fluidic composition including one or more volatile organic compounds (VOCs) such as (but not limited to) carbon dioxide (CO₂), carbon monoxide (CO), nitrogen dioxide (NO₂), one or more hydrocarbons including methane (CH₄), ethylene (C₂H₄), ethane (C₂H₆), or propane (C₃H₈), one or more acids including hydrochloric acid (HCl) or hydrofluoric acid (HF), one or more fluorinated hydrocarbons including phosphorus oxyfluoride, hydrogen cyanide (HCN), one or more aromatics including benzene (C₆H₆), toluene (C₇H₈), ethanol (C₂H₅OH), hydrogen, or one or more reduced sulfur compounds including thiols having a form of R—SH.

In some implementations, the carbon-based sensors 15-120 may include carbon particulates or 3D graphene structures that react with (or that can be configured to react with) analytes associated with batteries, for example, to determine whether a particular battery is leaking analytes that may be harmful or dangerous. In other implementations, the carbon-based sensors 15-120 may include carbon particulates or 3D graphene structures that react with (or that can be configured to react with) a group of analytes deemed to be harmful or dangerous, either individually or in combination with each other. For example, the carbon-based sensors 15-120 may be configured to produce detectable reactions when exposed to acetone and hydrogen peroxide to detect a presence of acetone peroxide (which is highly explosive). For another example, one or more of the carbon-based sensors 15-120 may be configured to detect a presence of triacetone triperoxide (TATP) or tri-cyclic acetone peroxide (TCAP), which are trimers for acetone peroxide.

In some implementations, each of the sensors 15-120 may be configured to react with a unique group of analytes. In some aspects, the sensors 15-120 may be functionalized with different materials configured to detect different analytes or different groups of analytes. In one implementation, a first sensor of the sensor array 15-110 may be functionalized with a first material configured to detect a presence of a first group of analytes, and one or more second sensors of the sensor array 15-110 may be functionalized with second materials configured to detect a presence of one or more corresponding second groups of analytes, where the second materials are different than each other and are different than the first material, and the second groups of analytes are unique subsets of the first group of analytes. For example, the first sensor may be configured to detect each of the five analytes 15-151-15-155, while each of the second sensors may be configured to detect only one of the five analytes 15-151-15-155. The first sensor may sense the environment for a relatively short period of time to provide a coarse detection of any of the analytes 15-151-15-155, and each of the second sensors may sense the environment for a relatively long period of time to confirm the presence of a respective one of the five analytes 15-151-15-155. In this way, the one or more second sensors 15-120 may be used to verify the detection of various analytes by the first sensor 15-120, thereby reducing or even eliminating false positives.

In other implementations, the sensors 15-120 may be configured to react with overlapping groups of analytes. In some other implementations, the sensors 15-120 may be configured to react with the same or similar groups of analytes.

The substrate 15-130 may be any suitable material. In some instances, the substrate may be paper or a flexible polymer. In other instances, the substrate 15-130 may be a rigid or semi-rigid material such as, for example, a printed circuit board.

FIG. 15-2 is an illustration 15-200 depicting the sensing device 15-100 of FIG. 15-1 coupled to a receptor 15-180, according to some implementations. The receptor 15-180 can be any suitable device, component, or mechanism capable or collecting, guiding, or steering analytes 151-155 present in a surrounding environment towards the sensing device 15-100. As shown, the receptor 15-180 includes an inlet 15-182 and a plurality of outlets 15-184. The inlet 15-182 may be configured to receive or attract the analytes 151-155 into the receptor 15-180, and the outlets 15-184 may be configured to steer the analytes 151-155 towards one or more exposed surfaces of the sensing device 15-100. In some implementations, each of the outlets 15-184 of the receptor 15-180 may be aligned with a corresponding sensor 15-120, for example, so that analytes 15-151-15-155 entering the receptor 15-180 can be released and exposed to each of the sensors 15-120 of the array 15-110. In this way, the receptor 15-180 may concentrate the analytes 151-155 on or near corresponding sensing materials 15-125 of the array 15-110, thereby increasing the likelihood of detection by the sensing device 15-100. For example, in instances for which the sensing device 15-100 is printed on a surface of a shipping package, a portion of the shipping package (e.g., a foldable flap) may be used as the receptor 15-180.

FIG. 15-3 is an illustration 15-300 depicting the sensing device 15-100 of FIG. 15-1 configured to detect a presence of analytes within or near a battery pack 15-310, according to some implementations. The battery pack 15-310 is shown to include a plurality of battery cells 15-320 arranged as a planar array on a substrate 15-312. One or more sensing devices 15-100 may be positioned near or coupled to a corresponding number of the battery cells 15-320 of the battery pack 15-310. In some implementations, a subset of the battery cells 15-320 may be associated with the sensing devices 15-100 such that the number of sensing devices 15-100 is less than the number of battery cells 15-320, for example, as depicted in the example of FIG. 15-3. In other implementations, each of the battery cells 15-320 may be associated with or coupled to a corresponding sensing device 15-100. In some instances, the sensors 15-120 of a respective sensing device 15-100 may be stacked on top of one another (such as in a vertical arrangement). In other instances, the sensors 15-120 of the respective sensing device 15-100 may be disposed next to one another (such as in a planar arrangement).

The sensing devices 15-100 may be configured to detect a presence of analytes 15-340 leaked from one or more of the battery cells 15-320 of the battery pack 15-310 in a manner similar to that described above with reference to FIG. 15-1. Specifically, each of the sensors 15-120 may be coupled between a corresponding pair of electrodes 15-121-15-122 and may include a plurality of 3D graphene-based sensing materials 15-125 configured to detect a presence of certain analytes (such as the analytes 15-151-15-155 of FIG. 15-1). In some implementations, the sensing materials 15-125 within different sensors 15-120 may be configured to detect the presence of different analytes or different groups of analytes. For example, in some instances, the sensing materials 15-125 of a first sensor 15-1201 may be functionalized with a first material configured to detect a presence of each analyte of a first group of analytes, and the sensing materials 15-125 of a second sensor 15-1202 may be functionalized with a second material configured to detect a presence of each analyte of a second group of analytes, where the second group of analytes is a subset of the first group of analytes. In other implementations, the sensing materials 15-125 within different sensors 15-120 may be configured to detect the presence of the same analytes or the same group of analytes.

In various implementations, each of the sensors 15-120 within a respective sensing device 15-100 may be configured to provide an output signal in response to detecting the presence of one or more analytes. In some implementations, the output signal may be a current generated in response to an alternating current provided to the respective sensor 15-120. In some instances, a difference between the alternating current and the output signal may be indicative of the presence or absence of the one or more analytes of the first group of analytes. In other implementations, the output signal may indicate a change in impedance of the corresponding sensor 15-120 caused by exposure to the one or more analytes. In some instances, a relatively small impedance change of the sensor 15-120 may indicate an absence of the one or more analytes, and a relatively large impedance change of the sensor 15-120 may indicate a presence of the one or more analytes.

In some other implementations, one or more of the sensing devices 15-100 may include an antenna (not shown for simplicity) configured to receive an electromagnetic signal from an external device, and the output signals may be frequency responses of the sensing materials 15-125 to the electromagnetic signal. For example, the frequency response of the sensing materials 15-125 of the first sensor 15-1201 may be indicative of the presence or absence of the first group of analytes, and the frequency response of the sensing materials 15-125 of the second sensor 15-1202 may be indicative of the presence or absence of the second group of analytes. In some aspects, the frequency responses may be based on resonant impedance spectroscopy (RIS) sensing.

In various implementations, the output signals generated by each sensing device 15-100 may indicate an operating mode of a corresponding battery cell 15-320 of the battery pack 15-310. In some implementations, the output signals may indicate a normal mode for the corresponding battery cell 15-320 based on an absence of analytes, may indicate a maintenance mode for the corresponding battery cell 15-320 based on the presence of analytes not exceeding a threshold level, or may indicate an emergency mode for the corresponding battery cell 15-320 based on the presence of analytes exceeding the threshold level. The output signals may also indicate a concentration level of each analyte detected by the sensing device 15-100.

FIG. 15-4 is an illustration 15-400 depicting the sensing device 15-100 of FIG. 15-1 configured to detect analytes in a shipping package 15-410, according to some implementations. The shipping package 15-410 is shown to include a surface 15-412 defining a volume within which one or more items (not shown for simplicity) can be contained. The defined volume of the shipping package 15-410 also includes a plurality of analytes 15-414 analytes 15-414 which can be, for example, one or more of the analytes 15-151-15-155 of FIG. 15-1. As shown, the sensing device 15-100 may be a label 15-430 that is printed onto the surface 15-412 of the shipping package 15-410. In various implementations, the label 15-430 may include a substrate 15-432, a plurality of carbon-based sensors 15-434 printed on the substrate, and one or more electrodes 15-436 printed on the substrate. The sensors 15-434, which may be examples of the carbon-based sensors 15-120 of FIG. 15-1, may be collectively configured to detect a presence of the analytes 15-414 within the shipping package 15-410.

In some implementations, each of the sensors 15-434 may be configured to react with a unique group of analytes in response to an electromagnetic signal 15-442 received from an external device 15-440 device 15-440. For example, a first sensor 15-4341 may be configured to detect the presence of a first group of analytes, and a second sensor 15-4342 may be configured to detect the presence of a second group of analytes that is a first subset of the first group of analytes. In one implementation, a third sensor 15-4343 may be configured to detect the presence of a third group of analytes that is a second subset of the first group of analytes. As discussed, the first sensor 15-4341 may be functionalized with a first material configured to react with the first group of analytes, the second sensor 15-4342 may be functionalized with a second material configured to react with the second group of analytes, and the third sensor 15-4343 may be functionalized with a third material configured to react with the third group of analytes. In this way, the second sensor 15-4342 may be used to confirm detection of the first subset of analytes by the first sensor 15-4341, and the third sensor 15-4343 may be used to confirm detection of the second subset of analytes by the first sensor 15-4341. In other implementations, one or more groups of sensors 15-434 may be configured to react with overlapping groups of analytes in response to the electromagnetic signal 15-442.

The electrodes 15-436, which may be examples of the electrodes 15-121-122 of FIG. 15-1, may be coupled to the sensors 15-434. In some implementations, each sensor 15-434 may be coupled between a corresponding pair of electrodes 15-436. The first electrode 15-436 of each electrode pair may be configured to receive the electromagnetic signal 15-442, and the second electrode 15-436 of each electrode pair may be configured to provide an output signal indicating whether the corresponding sensor 15-434 detected the presence of analytes.

In some implementations, each output signal may indicate a frequency response of a corresponding sensor 15-434 to the electromagnetic signal 15-442. For example, the frequency response of the first sensor 15-4341 may indicate the presence (or absence) of the first group of analytes within the shipping package 15-410, the frequency response of the second sensor 15-4342 may confirm the presence (or absence) of the second group of analytes, and the frequency response of the third sensor 15-4343 may confirm the presence (or absence) of the third group of analytes. In some instances, the first sensor 15-4341 may be exposed to the electromagnetic signal 15-442 for a relatively short period of time to provide a coarse indication of whether the analytes of the first group of analytes are present, and the second and third sensors 15-4342 and 15-4343 may be exposed to the electromagnetic signal 15-442 for a relatively long period of time to confirm indications of the presence of the second and third respective groups of analytes by the first sensor 15-4341. In this way, the sensors 15-4341-15-4343 can collectively reduce the number of false positives indicated by the sensing device 15-100.

In at least some implementations, an antenna (not shown for simplicity) may be printed on the substrate 15-432 and configured to drive an alternating current through the sensors 15-434 in response to the electromagnetic signal 15-442. Because the sensors 15-434 may be functionalized with different materials that can have different electrical and/or chemical characteristics, the resulting sensor output currents may indicate the presence (or absence) of different analytes. For example, in some instances, each output signal may indicate an impedance or reactance of a corresponding sensor 15-434 to the alternating current. The impedance or reactance of each sensor 15-434 can be measured and compared with a reference impedance or reactance to determine whether one or more analytes associated with the sensor 15-434 are present in the shipping package 15-410. In some instances, the reference impedances or reactance may be determined by driving the alternating current through the sensors 15-434 in the absence of all analytes, and measuring the impedances or reactance of the output signals from the sensors 15-434.

In some aspects, the sensors 15-434 may be juxtaposed in a planar arrangement on the substrate 15-432. In other instances, the sensors 15-434 may be stacked on top of one another in a vertical arrangement. In some implementations, the sensors 15-434 may form a permittivity gradient.

As discussed, the analyte sensing devices disclosed herein can be integrated into a product or package, such as on a cardboard box, or food package. The analyte sensing devices disclosed herein can be placed adjacent to a product or package and can detect analytes on or within the product or package. For example, the analyte sensing device can be integrated into or placed adjacent to a scale that is used to weigh shipping containers, and the analyte sensing device can be used to detect analytes on or within any shipping package being weighed by the scale. As another example, the analyte sensing device can be integrated into or placed adjacent to a component of a vehicle that is used to transport shipping containers, such as within a mail truck, and the analyte sensing device can be used to detect analytes on or within any shipping package being transported by the vehicle. As still further examples, the analyte sensing device can be integrated into a conveyor belt or mounted onto a portion of a mechanical conveyance device. Additionally, or alternatively, the analyte sensing device can be integrated into handling equipment, such as a robot arm, or handling apparel, such as gloves, etc., and the analyte sensing device can be used to detect analytes on or within any shipping package being conveyed or handled.

In one implementation, a fan or a suction device, such as a vacuum pump, may be used to direct environmental gasses (which may include one or more analytes) towards the analyte sensing device and/or into an enclosure containing the analyte sensing device. For example, the analyte sensing device can be placed into an enclosure, and a fan or vacuum pump can draw the surrounding environmental gasses into the enclosure such that any analytes present in the environmental gasses are exposed to the analyte sensing device. In another example, the analyte sensing device may be placed adjacent to a set of objects, such as shipping packages, mousepads, or other products, and can monitor for the presence of one or more analytes.

FIG. 15-5 is an illustration 15-500 depicting example reactions between one or more analytes and the sensor 15-120 of FIG. 15-1, according to some implementations. As discussed, the sensor 15-120 may include 3D graphene-based sensing materials 15-125 disposed on the substrate 15-130, and the sensing materials 15-125 may be functionalized with a material 15-126 configured to detect the presence of analytes 15-151-15-152. In some implementations, the sensing materials 15-125 may include a plurality of different graphene allotropes having one or more microporous pathways or mesoporous pathways. Although not shown for simplicity, a polymer may bind the plurality of different graphene allotropes to one another. In some instances, the polymer may include humectants configured reduce the susceptibility of the carbon-based sensors to humidity.

As shown, analytes 15-151-15-152 may take a variety of paths to penetrate and react with the sensing material 15-125. Specifically, inset 15-510 depicts the analytes 15-151-15-152 being adsorbed by the functionalized material 15-126 and/or various exposed surfaces of the sensing material 15-125. Inset 15-520 depicts a carbon particulate 15-522 from which the sensing material 15-125 may be formed. In some instances, a reactive chemistry additive (such as a salt dissolved in a carrier solvent) may be deposited on and within exposed surfaces, pores and/or pathways of the particulate carbon 15-522. In some instances, the reactive chemistry additives may be incorporated into the particulate carbon 15-522 to increase the sensitivity of the sensor 15-120 to one or more specific analytes.

FIG. 15-6 is a block diagram of an analyte detection system 15-600, according to some implementations. The analyte detection system 15-600 is shown to include an input circuit 15-610, a sensor array 15-620, a measurement circuit 15-630, and a controller 15-640. The input circuit 15-610 is coupled to the controller 15-640 and the sensor array 15-620, and may provide an interface through which currents, voltages, and electromagnetic signals can be applied to the sensor array 15-620. The sensor array 15-620, which may be one example of the sensor array 15-110 of FIG. 15-1, is shown to include eight carbon-based sensors 15-120₁-15-120₈ coupled between respective pairs of electrodes 15-121₁ and 15-122₁ through 15-121₈ and 15-122₈. In some instances, each of the first electrodes 15-121₁ 15-121₈ may be coupled to a corresponding terminal of the input circuit 15-610, and each of the second electrodes 15-122₁-15-122₈ may be coupled to a corresponding terminal of the measurement circuit 15-630. In other instances, each terminal of the input circuit 15-610 may be coupled to a corresponding group of the sensors 15-120₁-15-120₈.

The controller 15-640 may generate an excitation signal or field from which current levels, voltage levels, impedances, and/or frequency responses of the carbon-based sensors 15-120₁-15-120n can be measured or determined by the measurement circuit 15-630. For example, in some implementations, the controller 15-640 may be a current source configured to drive either a direct current or an alternating current through each of the sensors 15-120₁-15-120₈. In other implementations, the controller 15-640 may be a voltage source that can apply various voltages across the sensors 15-120₁-15-120₈ via corresponding pairs of electrodes 15-121 and 122. In some instances, the controller 15-640 can adjust the sensitivity of a respective sensor 15-120 to a particular analyte by changing the voltage applied across the respective sensor 15-120. For example, the controller 15-640 can increase the sensitivity of the respective sensor 15-120 by decreasing the applied voltage, and can decrease the sensitivity of the respective sensor 15-120 by increasing the applied voltage. In some other implementations, an antenna (not shown for simplicity) coupled to the sensor array 15-620 can receive one or more electromagnetic signals from an external device. In some aspects, the first electrodes 15-121₁-15-121₈ may be configured to receive the electromagnetic signals.

As discussed, the sensors 15-120₁-15-120₈ may include respective sensing materials 15-125₁-15-125₈ that can be functionalized with different materials configured to react with and/or detect different analytes or different groups of analytes. In some implementations, the sensors 15-120₁-15-120₈ may include cobalt in particulate form, and the sensing materials 15-125₁-15-125₈ may include carbon nano-onions (CNOs). Specifically, active sites on exposed surfaces of the CNOs may, in some aspects, be functionalized (such as through surface modification) with solid-phase cobalt (Co_{(S)}) (such as Co particles) and/or cobalt oxide (Co₂O₃), which reacts with available carbon on exposed surfaces of the CNOs. For example, the chemical reactions associated with using cobalt oxide to detect the presence of hydrogen peroxide (H₂O₂) may be expressed as:

$$2 {Co}^{\frac{3 +}{2 +}} Pc + {H}_{2} {O}_{2} \to 2 {Co}^{\frac{2 +}{+}} Pc + 2 {H}^{+} + {O}_{2} \qquad ( Eq . 1 )$$

$$2 {Co}^{\frac{2 +}{+}} Pc \to 2 {Co}^{\frac{3 +}{2 +}} Pc + 2 {e}^{-} \qquad ( Eq . 2 )$$

$${H}_{2} {O}_{2} \to 2 {H}^{+} + {O}_{2} + 2 {e}^{-} ( overall reaction ) \qquad ( Eq . 3 )$$

In addition, or the alternative, cobalt-based functionalization may be used to detect TATP according to the following chemical reaction:

$$TATP + {H}^{+} \to 3 {( {CH}_{3} )}_{2} CO + 3 {H}_{2} {O}_{2} \qquad ( Eq . 4 )$$

In other implementations, the presence of TATP may be detected based on the following steps or operations:
adsorption of TATP (50 ppb) onto exposed carbon surfaces (300-700 m²/g), which are acidic in nature by adding acid (such as HCl at an approximate 0.1m concentration level). Example acid treatment levels include 10 mg carbon (C) corresponding to 100 mg HCl at 0.1 m diluted in a suitable carrier solvent. Over time, the adsorbed HCl evaporates and protonates hydroxyl and/or carboxylic groups on exposed carbon surfaces to leave such surfaces in a relatively acidic state;
hydrolysis of TATP into acetone and peroxide;
performance of peroxide oxidation shown by Eq. (1)-(3) above; and
the generation of free electrons and associated observable changes in one or more electrical or chemical characteristics of the sensing device.

In some implementations, Cobalt decorated CNOs may provide the most selective and sensitive response to triacetone triperoxide (TATP) relative to other types of 3D graphene-based sensing materials. Applicant notes that since hydrogen peroxide has a chemical structure somewhat similar to triacetone triperoxide (TATP) or tri-cyclic acetone peroxide (TCAP), sensing devices configured to detect a presence of hydrogen peroxide can also be used to detect a presence of TATP.

The exact chemical reactivity and/or interactions between an analyte and exposed carbon surfaces of the materials 15-125₁-15-125₈ may depend on the type of analyte and the structure or organization of the corresponding materials 15-125₁-15-125₈. For example, certain analytes, such as hydrogen peroxide (H₂O₂) and TATP, may be detected by one or more oxidation-reduction (“redox”) type chemical reactions with metals decorated onto exposed carbon surface of the sensing materials 15-125₁-15-125₈. In some implementations, some of the sensing materials 15-125₁-15-125₈ may be prepared or created to include free amines, which may react with electronic deficient nitroaromatic analytes, such as TNT and DNT.

The measurement circuit 15-630 may measure the output signals provided by the sensors 15-120₁-15-120₈ to determine whether certain analytes are present in the surrounding environment. For example, when the sensor array 15-120 is pinged with an electromagnetic signal (e.g., received from an external device such as the device 15-440 of FIG. 15-4), the measurement circuit can measure the frequency responses of the sensors 15-120₁-15-120₈, and compare the measured frequency responses with one or more reference frequency responses. If the measured frequency response of a sensor 15-120 matches a particular reference frequency response, then the measurement circuit 15-630 may indicate a presence of analytes associated with the particular reference frequency response. Conversely, if the measured frequency response of the sensor 15-120 does not match any of the reference frequency responses, then the measurement circuit 15-630 may indicate an absence of analytes associated with the particular reference frequency response.

For another example, application of an alternating current to the sensor array 15-120 may cause one or more electrical and/or chemical characteristics of the sensors 15-120₁-15-120₈ to change (e.g., to increase or decrease). The measurement circuit 15-630 can detect the resultant changes in the electrical and/or chemical characteristics of the sensors 15-120₁-15-120₈, and can determine whether certain analytes are present based on the changes. In some implementations, the measurement circuit 15-630 can measure the output currents of sensors 15-120₁-15-120₈ caused by the alternating current, and can compare the measured output currents with one or more reference currents to determine whether certain analytes are present. Specifically, if the measured output current of a sensor 15-120 matches a particular reference current, then the measurement circuit 15-630 may indicate the presence of analytes associated with the particular reference current. Conversely, if the measured output current of the sensor 15-120 does not match any of the reference currents, then the measurement circuit 15-630 may indicate an absence of analytes associated with the particular reference current.

In other implementations, the measurement circuit 15-630 can measure the impedances or reactance of the sensors 15-120₁-15-120₈ to the alternating current, and can compare the measured impedances or reactance with one or more reference impedances or reactance to determine whether certain analytes are present. Specifically, if the measured impedance or reactance of a sensor 15-120 matches a reference impedances or reactance, then the measurement circuit 15-630 may indicate the presence of analytes associated with the reference impedances or reactance. Conversely, if the measured impedance or reactance of the sensor 15-120 does not match any of the reference impedances or reactance, then the measurement circuit 15-630 may indicate an absence of analytes associated with the reference impedances or reactance.

FIG. 15-7A shows another sensor array 15-700A, according to some implementations. As shown, the sensor array 15-700A includes a plurality of sensors 15-701-15-704 disposed in a planar arrangement, with each of the sensors 15-701-15-704 including a different carbon-based sensing material. In some aspects, the sensors 15-701-15-704 may be examples of the sensors 15-120 of FIGS. 15-1-15-3 and FIGS. 15-5-15-6. In other aspects, the sensors 15-701-15-704 may be examples of the sensors 15-434 of FIG. 15-4. Although the example 15-700A of FIG. 15-7A shows four sensors 15-701-15-704 arranged in a 2-row by 2-column array, in other implementations, the other numbers of sensors can be disposed in other suitable arrangements.

The sensors 15-701-15-704 may include routing channels between individual deposits of the carbon-based sensing materials. These routing channels may provide routes through which electrons can flow through the sensors 15-701-15-704. The resulting currents through the sensors 15-701-15-704 can be measured through ohmic contact with the respective electrode pairs E1-E4. For example, a measurement M₁ of the first sensor 15-701 sensor 15-701 can be taken via electrode pair E1, a measurement M₂ of the second sensor 15-702 can be taken via electrode pair E2, a measurement M₃ of the third sensor 15-703 can be taken via electrode pair E3, and a measurement M₄ of the fourth carbon-based sensor 15-704 can be taken via electrode pair E4.

In various implementations, each of the sensors 15-701-15-704 can be configured to react with and/or to detect a corresponding analyte or group of analytes. For example, the first sensor 15-701 sensor 15-701 can be configured to react with or detect a first group of analytes in a coarse-grained manner, and the second sensor 15-702 can be configured to react with or detect a subset of the first group of analytes in a fine-grained manner. In some instances, the sensors 15-701-15-704 can be printed onto a substrate using different carbon-based inks. Ohmic contact points can be used to capture the measurements M₁-M₄, either concurrently or sequentially.

FIG. 15-7B shows another sensor array 15-700B, according to some implementations. The sensor array 15-700B includes a plurality of carbon-based sensors 15-701-15-704 stacked on top of one another in a vertical or stacked arrangement. In some aspects, the sensors 15-701-15-704 may be examples of the sensors 15-120 of FIGS. 15-1-15-3 and FIGS. 15-5-15-6. In other aspects, the sensors 15-701-15-704 may be examples of the sensors 15-434 of FIG. 15-4. The sensors 15-701-15-704 (and their respective sensing materials) can be sequentially deposited upon one another to form the stacked array. In some instances, separators (not shown for simplicity) can be provided between the sensors 15-701-15-704. As discussed, the sensors 15-701-15-704 may be functionalized with different materials and/or may include different types of carbon-based sensing materials that can be printed in successive layers onto a substrate or label.

As the demand for low-cost analyte sensors continues to increase, it is increasingly important to reduce or even eliminate the need for electronic components in analyte sensors. For example, the high cost of electronic components typically found in conventional analyte sensors render their widespread deployment in shipping containers, packages, and envelopes impractical. As such, some implementations of the subject matter disclosed herein may provide a cost-effective solution to the long-standing problem of monitoring large numbers of shipping containers, packages, and envelopes for the presence of harmful chemicals and gases such as, for example, the various analytes described herein.

FIG. 15-7C is an illustration 15-700C depicting an ink-jet or bubble-jet print head 15-720 printing various sensing devices disclosed herein onto the surface of a shipping container, package, or envelope, according to some implementations. Specifically, the illustration 15-700C depicts a process by which multiple layers of different carbon-based sensing materials 15-711-15-714 can be printed onto a substrate 15-710. As shown, the print head 15-720 can print a first layer 15-711 of carbon-based sensing materials onto the substrate 15-710 using a first carbon-based ink 15-721, can print a second layer 15-712 of carbon-based sensing materials onto the substrate 15-710 using a second carbon-based ink 15-722, can print a third layer 15-713 of carbon-based sensing materials onto the substrate 15-710 using a third carbon-based ink 15-723, and can print a fourth layer 15-714 of carbon-based sensing materials onto the substrate 15-710 using a fourth carbon-based ink 15-724. In some instances, the carbon-based inks 15-721-15-724 may be different from one another, for example, such that the resulting sensing material layers 15-711-15-714 are configured to react with and/or detect different analytes or different group of analytes. The print head 15-720 can also print electrodes E1-E4 for the different sensing material layers 15-711-15-714, respectively, using an ohmic-based ink 15-725. Ohmic contacts can be printed onto the substrate 15-710 and/or portions of the sensing material layers 15-711-15-714 using multiple passes of the multi-jet print head 15-720. In some implementations, the sensing device may include vias through which the resulting electrodes E1-E4 can be accessed. In other implementations, other suitable mechanisms can be used to provide ohmic contacts for the electrodes E1-E4.

FIG. 15-7D is an illustration 15-700D depicting the print head 15-720 printing various sensing devices disclosed herein onto the surface of a shipping container, package, or envelope, according to other implementations. Specifically, the illustration 15-700D depicts a process by which multiple layers of different carbon-based sensing materials 15-711-15-714 can be printed onto a substrate 15-710 in a pyramid arrangement. The illustration 15-700D also depicts ohmic contacts 15-705 printed on the sensing material layers 15-711-15-714 using an ohmic-based ink 15-725. In some aspects, the different sizes and different exposed surface areas of the sensing material layers 15-711-15-714 may cause the respective sensors to have different electrical and/or chemical characteristics, which in turn may configured the respective sensors to react with and/or detect different types of analytes.

Further details pertaining to various carbon-based sensing materials, tunings, and calibration techniques that can be used to form carbon-based sensors disclosed herein are summarized below in Table 15-1.

TABLE 15-1

| Components | Tuning | Sensitivity | Calibration |
|---|---|---|---|
| Different carbon | Select carbon | Surface area of | Response of the selected |
| types and/or different | functionalization | carbon-based | carbon functionalization |
| carbon decorations | materials to detect | sensor | materials to the selected |
| | selected analytes | | analytes |
| Physical dimensions | Select size and/or | Surface area of | Sensitivity is based on |
| of the sensing | aspect ratio of exposed | carbon-based | physical dimensions and |
| material | portion of sensor | sensor | characteristics of |
| | | | carbon-based sensor |
| Adjacency or | Select distance between | Select distance | Calibrate based on test |
| proximity to other | sensors to reduce | between sensors to | sample over a range |
| carbon-based sensing | overlapping response | reduce overlapping | conditions |
| materials | signals | response signals | |
| Different permittivity | Tune permittivity based | Select distance | Calibrate based on test |
| of the different | on sensor | between sensors to | sample over a range |
| materials | material/functionalization | reduce overlapping | conditions |
| | | response signals | |

As discussed, different materials may resonate at different frequencies, and many materials may resonate at different frequencies depending on whether one or more certain analytes are present. In some implementations, the permittivity of carbon-based sensing materials described herein can be modified by exposing the materials to ultraviolet (UV) radiation.

FIG. 15-7E is an illustration 15-700E depicting UV radiation emitted towards the sensor 15-701. As shown, a UV beam source 15-753 may be used to shower the sensor 15-701 with UV radiation. The power and wavelength of the UV radiation can be controlled by a power control unit 15-751 and a wavelength control unit 15-752, respectively. In some implementations, adjusting the power level and/or wavelength of UV radiation can change the permittivity of each of the sensing material layers 15-711-15-714. That is, after bombarded with the UV radiation, each of the sensing material layers 15-711-15-714 may resonate at a different frequency. In some aspects, the different permittivity of the sensing material layers 15-711-15-714 may collectively a permittivity gradient 15-725. The permittivity gradient 15-725 may correspond to a stair shaped gradient 15-761, a linearly shaped gradient 15-762, or a curvilinearly shaped gradient 15-763.

FIG. 15-8 shows a flow chart 15-800 depicting an example operation for fabricating at least some of the sensing devices disclosed herein, according to some implementations. In various implementations, the permittivity of a carbon-based sensing material can be altered to cause a particular resonance signature in the carbon-based sensing material when exposed to certain analytes. In some cases, different portions of the carbon-based sensing material may be configured to have different permittivity values specifically selected to cause particular resonance frequencies and/or or resonance signatures. In particular, it is sometimes desired that a first portion of the carbon-containing material with a first permittivity that is tuned to resonate with a particular resonance signature when the first portion of the carbon-containing material has imbibed a first analyte of interest, whereas a second portion of the carbon-containing material has a second permittivity that is tuned to resonate with a particular resonance signature when the second portion of the carbon-containing material has imbibed a second analyte of interest.

Formation of different portions of the carbon-containing material having different permittivity values can be accomplished using a combination of masking and UV treatments. At block 15-802, a carbon-containing material is deposited onto a substrate or electrode 15-811. At block 15-804, a UV-opaque mask is deposited or printed on top the carbon-containing material. At block 15-806, the carbon-containing material is activated, for example, via bombardment by UV photons. This results in a first portion 15-8121 of the carbon-containing material having a first permittivity, and a second portion 15-8122 of the carbon-containing material having a second permittivity different than the first permittivity. At block 15-808, the mask can be washed away, ablated, or otherwise removed. Two or more of the resulting analyte-sensing devices can be used as a multi-element, multi-analyte sensor and/or as a high-sensitivity analyte sensor. In addition, or in the alternative, the resulting analyte-sensing devices can be exposed to an additional bombardment of UV photons at block 15-810, for example, to further alter portions of the carbon-containing material previously beneath the UV-opaque mask.

Some example alternative implementations are summarized below in Table 15-2:

TABLE 15-2

| Manufacturing Process Aspect(s) | Result(s) |
|---|---|
| Add a hardener or binder to the carbon- | UV treatment causes curing and hardening to a |
| containing materials | controllable degree (such as to be more rigids |
| | or more flexible) |
| Use the UV photon to ablate some of the | Form patterns in the carbon-containing |
| carbon-containing material | material that absorb an analyte into the carbon- |
| | containing material and/or that increase |
| | coupling between the carbon-containing |
| | material and the electrode. |
| Deposit a slurry of carbon-containing materials | Low cost, high-volume manufacture of |
| over a sheet of conductive, semi-conductive, or | analyte-sensing devices. |
| non-conductive material. | |
| Add metallic and/or semiconducting and/or | Facilitates permeation of certain analytes into |
| dielectric, and/or polymeric materials to the | the matrix and/or tunes the matrix to be |
| carbon-containing materials (such as to the | sensitive to particular analytes. |
| slurry) to form an open-pore matrix. | |
| Add metallic and/or semiconducting and/or | Tunes the matrix to be sensitive to particular |
| dielectric, and/or polymeric materials to the | analytes and/or facilitates permeation of certain |
| carbon-containing materials (such as to the | analytes into the matrix. |
| slurry) to form an open-pore matrix. | |
| Maintain low temperatures during processing. | A void loss of conductivity that may occur at |
| | higher temperatures (such as when polymers |
| | unwantedly coat metallics). |

FIG. 15-9 shows another sensor array 15-900, according to some implementations. The sensor array 15-900 includes a plurality of layers 15-911-15-914 of individually-functionalized carbon-containing materials. As shown, the layers 15-911-15-914 are successively disposed to form a stack of layers, with the first layer 15-911 disposed on the substrate 15-910. Each layer is formed of a corresponding individually-functionalized carbon-containing matrix (such as carbon matrix1, carbon matrix2, carbon matrix3, carbon matrix4), wherein each individually-functionalized carbon-containing matrix includes a respective additive A-D). The combinations of carbon-containing matrices and additive may be selected based on the particular combination's sensitivity to a particular analyte of interest.

In forming the analyte sensor array, the different layers can be deposited using any known technique. Furthermore, each of the different layers can be configured to be of a particular thickness. Strictly as one example, and as shown, a first deposited layer can have a first thickness 15-924 in a first range (such as 10 nm-100 nm, whereas another deposited layer can have a thickness in a different range (such as 500 nm-1,000 nm), and so on. The particular thickness of a particular layer can be selected based upon any combination of:
characteristics of the additive for that particular layer, and/or
characteristics of the analyte of interest, and/or
innate binary-tertiary interactions by and between the constituents of the layer.

In some implementations, the open pore structure of carbon-based sensing materials disclosed herein may allow certain analytes to more easily penetrate the materials and/or to more easily interact with carbon matrices within the materials. As such, these open pores may increase the sensitivity of sensors disclosed herein to analytes than conventional analyte detection systems.

FIG. 15-10A shows an example sensor configuration 15-1000A, according to some implementations. The sensor configuration 15-1000A includes mappings between sensors of an analyte detection system and various analytes, according to some implementations. For example, the 3D graphene-based sensing materials of the carbon-based sensors 15-120 of FIG. 15-1 may be or include the carbon recipes shown in the sensor configuration 15-1000A. That is, in a configuration in which the sensor array 15-120 includes 8 carbon-based sensors 15-120, each sensor may have a corresponding carbon recipe as shown by the example sensor configuration 15-1000A. For example, a first sensor may be cobalt oxide (Co₂O₃) decorated CNO and produce a percentage change in current (% ΔI) over initial current (I₀) and/or percentage increase in measured impedance of 9.26244%, and so on. In this way, the carbon recipes of the sensor configuration 15-1000A may be used to configure the carbon-based sensors to detect and identify different analytes (such as TATP, DNT, H₂S, and so on), even at relatively low concentration levels, based on their respective chemical fingerprints. As such, the sensing devices disclosed herein may be able to detect relatively low concentrations of analytes and/or other chemical threat agents, even in the presence of common interferents.

FIG. 15-10B shows another example sensor configuration 15-1000B, according to some implementations. The sensor configuration 15-1000B may be similar to the sensor configuration 15-1000A of FIG. 15-10A, for example, such that:

Sensor No. 1: Carbon #29, corresponding to carbon nano-onion (CNO) oxides produced in a thermal reactor; cobalt(II) acetate (C₄H₆CoO₄), the cobalt salt of acetic acid (often found as tetrahydrate Co(CH₃CO₂)₂·4H₂O, abbreviated Co(OAc)₂·4H₂O, is flowed into the thermal reactor at a ratio of approximately 59.60 wt % corresponding to 40.40 wt % carbon (referring to carbon in CNO form), resulting in the functionalization of active sites on the CNO oxides with cobalt, showing cobalt-decorated CNOs at 15,000× and 100,000× levels, respectively; suitable gas mixtures used to produce Carbon #29 and/or the cobalt-decorated CNOs may include the following steps:
Ar purge 0.75 standard cubic feet per minute (scfm) for 30 min;
Ar purge changed to 0.25 scfm for run;
temperature increase: 25° C. to 300° C. 20 mins; and
temperature increase: 300°−500° C. 15 mins.

Sensor No. 2: corresponding to TGJM (thermal graphene jet milled; thermal reactor carbon unfunctionalized) as shown in FIG. 15-11A.

Sensor No. 3: Carbon #19, corresponding to “DXR” (as characterized by FIGS. 15-5A and/or 15-5B) type or configuration carbons produced in a microwave reactor (such as a reactor coupled to a microwave source such that microwave energy propagates through the reactor exciting carbon-containing gases and/or plasmas inside the reactor); silver acetate (CH₃CO₂Ag), a white, crystalline solid particulate substance suspended in carrier gas to create silver acetate vapor, is flowed into the microwave reactor at a ratio of approximately 58.18 wt % corresponding to 41.82 wt % carbon (referring to carbon in DXR form), resulting in the functionalization of active sites on the DXR configuration carbons with silver as substantially shown in FIG. 15-11D (in undecorated form) and/or in FIG. 15-11G (showing actual decoration with cobalt instead of silver). Example gas mixtures used to produce Carbon #19 and/or the silver-decorated DX R carbons substantially shown in FIGS. 15-11D and 15-11G may include the following steps:
flow of carrier gas over DXR carbon structures at a volume ratio of 6.7% H₂ per 93.3% Ar for approximately 1 minute and 8 seconds

Sensor No. 4: CNO (carbon nano-onion; thermal reactor carbon unfunctionalized) as shown in FIG. 15-11B.

Sensor No. 5: Carbon #16, corresponding to “DXR” (as characterized by FIGS. 15-5A and/or 15-5B) type or configuration carbons produced in a microwave reactor; iron (II) acetate, a white solid particulate substance suspended in carrier gas to create iron acetate vapor, is flowed into the microwave reactor at a ratio of approximately 65.17 wt % corresponding to 34.83 wt % carbon (referring to carbon in DX R form), resulting in the functionalization of active sites on the DXR configuration carbons with silver as substantially shown in FIG. 15-11D (in undecorated form) and/or in FIG. 15-11G (showing actual decoration with cobalt instead of iron). Example gas mixtures used to produce Carbon #16 and/or the iron-decorated DX R carbons substantially shown in FIGS. 15-11D and 15-11G may include the following steps:
flow of carrier gas over DXR carbon structures at a volume ratio of 6.7% H₂ per 93.3% Ar for approximately 1 minute and 13 seconds.

Sensor No. 6: Carbon #1, corresponding to “Anvel” (as characterized by FIG. 15-7C) type or configuration carbons produced in a microwave reactor; platinum(II) bis(acetylacetonate), a coordination compound with the formula Pt(O₂C₅H₇)₂, abbreviated Pt₂, is flowed as a particulate dispersed in carrier gas to create platinum(II) bis(acetylacetonate) vapor, is flowed into the microwave reactor at a ratio of approximately 76.62 wt % corresponding to 23.38 wt % carbon (referring to carbon in Anvel form), resulting in the functionalization of active sites on the Anvel configuration carbons with platinum as substantially shown in FIG. 15-11C (in undecorated form); suitable gas mixtures used to produce Carbon #1 and/or the undecorated Anvel carbons substantially shown in FIGS. 15-11C may include the following steps:
flow of carrier gas over Anvel carbon structures at a volume ratio of 6.7% H₂ per 93.3% Ar for approximately 15 minutes

Sensor No. 7: Carbon #6, corresponding to “Anvel” (as characterized by FIG. 15-7C) type or configuration carbons produced in a microwave reactor; palladium (II) acetate, a chemical compound of palladium described by the formula [Pd(O₂CCH₃)₂]ₙ, abbreviated [Pd(OAc)₂]ₙ, is flowed as a particulate dispersed in carrier gas to create palladium (II) acetate vapor, is flowed into the microwave reactor at a ratio of approximately 65.17 wt % corresponding to 34.83 wt % carbon (referring to carbon in Anvel form), resulting in the functionalization of active sites on the Anvel configuration carbons with platinum as substantially shown in FIG. 15-11C (in undecorated form). Example gas mixtures used to produce Carbon #6 and/or the palladium-decorated Anvel carbons substantially shown in FIG. 15-11C may include the following steps:
flow of carrier gas over Anvel carbon structures at a volume ratio of 6.7% H₂ per 93.3% Ar for approximately 15 minutes.

Sensor No. 8:1,3-diaminonaphthalene complexed to TG-JM, such as that shown in FIG. 15-11A, to produce an organically modified carbon.

FIGS. 15-11A-15-11G show illustrations of various structured carbon materials that can be used in the sensing devices disclosed herein, according to some implementations. For example, FIG. 15-11A shows a micrograph 15-1100A of thermogravimetric (TG) carbons, according to various implementations. FIG. 15-11B shows a micrograph 15-1100B of undecorated CNOs, according to various implementations. FIG. 15-11C shows a micrograph 15-1100C of Anvel carbons, according to various implementations. FIG. 15-11D shows a micrograph 15-1100D of DXR carbons, according to various implementations. FIG. 15-11E shows a micrograph 15-1100E of cobalt decorated CNOs at a magnification level of 15,000×, according to various implementations. FIG. 15-11F shows a micrograph 15-1100F of cobalt decorated CNOs at a magnification level of 100,000×, according to various implementations. FIG. 15-11G shows a micrograph 15-1100G of a cobalt decorated DXR carbons, according to various implementations.

In contrast to a conventional 2D graphene material, the 3D graphene sensing materials disclosed by the present implementations may be designed to have a convoluted 3D structure to prevent graphene restacking, avoiding several drawbacks of using 2D graphene as a sensing material. This process also increases the areal density of the materials, yielding higher analyte adsorption sites per unit area, thereby improving chemical sensitivity, as made possible by a corresponding library of carbon allotropes used to customize the sensor arrays disclosed herein to chemically fingerprint leaked analytes for multiple applications.

The structured carbon materials shown in FIGS. 15-11A-15-11G may be produced using flow-through type microwave plasma reactors configured to create pristine 3D graphene particles continuously from a hydrocarbon gas at near atmospheric pressures. Operationally, as the hydrocarbon flows through a relatively hot zone of a plasma reactor, free carbon radicals may be formed that flow further down the length of the reactor into the growth zone where 3D carbon particulates (based on multiple 2D graphenes joined together) are formed and collected as fine powders. The density and composition of the free-radical carbon-inclusive gaseous species may be tuned by gas chemistry and microwave (M W) power levels. By controlling the reactor process parameters, these reactors may produce carbons with a wide, yet tunable, range of morphologies, crystalline order, and sizes (and distributions). For example, possible sizes and distributions may range from flakes (few 100 nm to μm wide and few nm thin) to spherical particles (10s of nm in diameter) to graphene clusters (10s of μm). The 3D nature of the materials prevents agglomeration effectively allowing for the materials to be disseminated as un-agglomerated particles. As a result, highly responsive and selective sensing materials can be produced. Graphene, an atomically thin two dimensional (2D) material, has many advantageous properties for sensing, including outstanding chemical and mechanical strength, high carrier mobility, high electrical conductivity, high surface area, and gate-tunable carrier density.

To improve the chemical selectivity, the 3D graphenes of the presently disclosed graphenes may be functionalized with various reactive materials in such a manner that the binding of target molecules and the carbon may be optimized. This functionalization step along with the ability to measure the complex impedance of the exposed sensor may be critical for efficient and selective detection of analytes. For example, different metal nanoparticles or metal oxide nanoparticles may be decorated on the surface of 3D graphenes to selectively detect hydrogen peroxide (a TATP degradation product) as peroxides are known to react with different metals. Further, nanoparticle decorated graphene structures may act synergistically to offer desirable and advantageous properties for sensing applications.

FIGS. 15-12A-15-12F show example frequency responses of resonance impedance sensors to various analytes, according to some implementations. Specifically, FIG. 15-12A shows an example frequency response 15-1200A of sensors 15-120 to alongside a baseline or reference frequency response. Specifically, FIG. 15-12A shows an example frequency response 15-1200A of sensors 15-120 to acetone alongside a baseline or reference frequency response. FIG. 15-12B shows an example frequency response 15-1200B of sensors 15-120 to acetonitrile alongside a baseline or reference frequency response. FIG. 15-12C shows an example frequency response 15-1200C of sensors 15-120 to Ethanol alongside a baseline or reference frequency response. FIG. 15-12D shows an example frequency response 15-1200A of sensors 15-120 to isopropanol alongside a baseline or reference frequency response. FIG. 15-12E shows an example frequency response 15-1200E of sensors 15-120 to water alongside a baseline or reference frequency response. FIG. 15-12F shows an example frequency response 15-1200F of sensors 15-120 to xylenes alongside a baseline or reference frequency response.

FIG. 15-13A is a graph 15-1300A depicting the real (Z′) impedance component of example frequency responses of sensors 15-120 to Acetone, Ethanol (EtOH), water, and hydrogen peroxide (H₂O₂) alongside a baseline or reference frequency response, according to some implementations. FIG. 15-13B is a graph 15-1300B depicting the imaginary (Z″) impedance component of example frequency responses of sensors 15-120 to Acetone, Ethanol (EtOH), water, and hydrogen peroxide (H₂O₂) alongside a baseline or reference frequency response, according to some implementations.

FIG. 15-14A shows an example frequency response of sensors 15-120 to hydrogen peroxide alongside a baseline or reference frequency response, according to some implementations. FIG. 15-14B shows example frequency responses of sensors 15-120 to acetone and water, according to some implementations. FIG. 15-14C shows example frequency responses to ethanol and ammonia, according to some implementations.

One innovative aspect of the subject matter described in this disclosure may be implemented as a sensing device for detecting analytes. The sensing device may include a substrate and a sensor array. The sensor array may be arranged on the substrate, and may include a plurality of carbon-based sensors. In some implementations, a first carbon-based sensor disposed between a first pair of electrodes may be configured to detect a presence of each analyte of a first group of analytes, and a second carbon-based sensor disposed between a second pair of electrodes may be configured to detect a presence of each analyte of a second group of analytes, where the second group of analytes is a subset of the first group of analytes. In some instances, the first group of analytes may include at least twice as many different analytes as the second group of analytes. In some implementations, the first carbon-based sensor may be configured to generate a first output signal in response to detecting the presence of one or more analytes of the first group of analytes, and the second carbon-based sensor may be configured to generate a second output signal in response to confirming the presence of the one or more analytes detected by the first carbon-based sensor. In one implementation, the first and second output signals may be currents based at least in part on an alternating current applied to the first and second carbon-based sensors. In some instances, a ratio of the current of the first output signal and the alternating current may be indicative of a concentration of at least one of the detected analytes, and a ratio of the current of the second output signal and the alternating current may be indicative of a concentration of at least one of the confirmed analytes.

In other implementations, the first and second output signals may be indicative of the impedances of the first and second carbon-based sensors, respectively. In some aspects, the first output signal may indicate a change in impedance of the first carbon-based sensor caused by exposure to one or more analytes of the first group of analytes, and the second output signal may indicate a change in impedance of the second carbon-based sensor caused by exposure to one or more analytes of the second group of analytes. In some other implementations, the first and second output signals may indicate frequency responses of the first and second carbon-based sensors, respectively. In some instances, the frequency response of the first carbon-based sensor may be indicative of the presence or absence of each analyte of the first group of analytes, and the frequency response of the second carbon-based sensor may be indicative of the presence or absence of each analyte of the second group of analytes. The frequency responses may be based on electrochemical impedance spectroscopy (EIS) sensing or resonant impedance spectroscopy (RIS) sensing.

In various implementations, the first carbon-based sensor may be functionalized with a first material configured to react with each analyte of the first group of analytes, and the second carbon-based sensor may be functionalized with a second material configured to react only with the analytes of the second group of analytes. In some instances, the first material may be cobalt-decorated carbon nano-onions (CNOs) configured to detect a presence of one or more of triacetone triperoxide (TATP), toluene, ammonia, or hydrogen sulfide (H₂S), and the second material may be iron-decorated three-dimensional (3D) graphene-inclusive structures configured to confirm the presence of toluene.

The substrate may be paper, a flexible polymer, or other suitable material. In some implementations, the substrate and the sensor array may be integrated within a label configured to be removably printed onto a surface of a package or container. In some aspects, each of the carbon-based sensors may be printed on the substrate using a different carbon-based ink, and the pairs of electrodes may be printed on the substrate using an ohmic-based ink. In some instances, the first and second carbon-based sensors may be stacked on one another. In other instances, the first and second carbon-based sensor may be disposed next to one another.

In some implementations, each of the carbon-based sensors may include a plurality of different graphene allotropes. In some aspects, the different graphene allotropes of a respective carbon-based sensor may include one or more microporous pathways or mesoporous pathways. Each of the carbon-based sensors may include a polymer configured to bind the plurality of different graphene allotropes to one another. The polymer may include humectants configured reduce a susceptibility of a respective carbon-based sensor to humidity.

Another innovative aspect of the subject matter described in this disclosure may be implemented as a sensing device for detecting analytes within a package or container. In various implementations, the sensing device may include a substrate, one or more electrodes, and a sensor array. The sensor array may be disposed on the substrate, and may include a plurality of carbon-based sensors coupled to the one or more electrodes. In some implementations, the carbon-based sensors may be configured to react with unique groups of analytes in response to an electromagnetic signal received from an external device. In some instances, the carbon-based sensors may be configured to resonate at different frequencies in response to the electromagnetic signal. Each of the one or more electrodes may be configured to provide an output signal indicating whether a corresponding carbon-based sensor detected one or more analytes in a respective group of the unique groups of analytes. In some instances, each output signal may indicate an impedance or reactance of the corresponding carbon-based sensor.

In addition, or in the alternative, a first frequency response of the first carbon-based sensor to the electromagnetic signal may be indicative of the presence or absence of the analytes of the first group of analytes within the package or container, and a second frequency response of the second carbon-based sensor to the electromagnetic signal may be indicative of the presence or absence of the analytes of the second group of analytes within the package or container. In some instances, the first frequency response may be based at least in part on exposure of the first carbon-based sensor to the electromagnetic signal for a first period of time, and the second frequency response may be based at least in part on exposure of the second carbon-based sensor to the electromagnetic signal for a second period of time that is longer than the first period of time. In some instances, the second period of time is at least twice as long as the first period of time. The first and second frequency responses may be based on resonant impedance spectroscopy (RIS) sensing.

In various implementations, a first carbon-based sensor may be functionalized with a first material configured to detect the presence of each analyte of a first group of analytes, and a second carbon-based sensor may be functionalized with a second material configured to detect the presence of each analyte of a second group of analytes. The second group of analytes may be a subset of the first group of analytes, and the second material may be different than the first material. In some aspects, the first group of analytes may include at least twice as many different analytes as the second group of analytes. In some instances, the first material may be cobalt-decorated carbon nano-onions (CNOs) configured to detect the presence of one or more of triacetone triperoxide (TATP), toluene, ammonia, or hydrogen sulfide (H₂S), and the second material may be iron-decorated three-dimensional (3D) graphene-inclusive structures configured to confirm the presence of toluene. In various implementations, a third carbon-based sensor may be functionalized with a third material configured to detect the presence of each analyte of a third group of analytes, where the third group of analytes may be another subset of the first group of analytes, and the third material may be different than the first and second materials.

In some implementations, at least two of the carbon-based sensors may be juxtaposed in a planar arrangement on the substrate. In other implementations, the carbon-based sensors may be stacked on top of one another in a vertical arrangement. For example, in one implementation, the carbon-based sensors may form a permittivity gradient. In some aspects, a single electrode may be configured to provide an output signal indicating whether the stacked carbon-based sensors detected one or more analytes. The single electrode may also be configured to provide the output signal to the external device.

The substrate may be paper, a flexible polymer, or other suitable material. In some implementations, the substrate and the sensor array may be integrated within a label that can be removably printed on a surface of the package or container. In some aspects, each of the carbon-based sensors may be printed on the substrate using a different carbon-based ink, and the one or more electrodes may be printed on the substrate using an ohmic-based ink. In some implementations, each of the carbon-based sensors may include a plurality of different graphene allotropes. In some aspects, the different graphene allotropes of a respective carbon-based sensor may include one or more microporous pathways or mesoporous pathways. Each of the carbon-based sensors may include a polymer configured to bind the plurality of different graphene allotropes to one another. The polymer may include humectants configured reduce a susceptibility of a respective carbon-based sensor to humidity.

Another innovative aspect of the subject matter described in this disclosure may be implemented as a sensing device for monitoring a battery pack. The sensing device may include a substrate and a plurality of carbon-based sensors disposed on the substrate. Each of the carbon-based sensors may be coupled between a corresponding pair of electrodes. In some implementations, the 3D graphene-based sensing materials of a first carbon-based sensor may be functionalized with a first material configured to detect a presence of each analyte of a first group of analytes, and the 3D graphene-based sensing materials of a second carbon-based sensor may be functionalized with a second material configured to detect a presence of each analyte of a second group of analytes. In some aspects, the second group of analytes is a subset of the first group of analytes, and the group of analytes may include at least twice as many different analytes as the second group of analytes. In some instances, the first and second carbon-based sensors may be stacked on top of one another. In other instances, the first and second carbon-based sensors may be disposed next to one another. In some implementations, the carbon-based sensors may be carbon-based inks printed on the substrate. In some instances, the first carbon-based sensor may be a first carbon-based ink, and the second carbon-based sensor may be a second carbon-based ink different than the first carbon-based ink.

The first carbon-based sensor may be configured to generate a first output signal in response to detecting the presence of one or more analytes of the first group of analytes, and the second carbon-based sensor may be configured to generate a second output signal in response to confirming the presence of the one or more analytes detected by the first carbon-based sensor. In some implementations, the sensing device may include an input terminal to receive an alternating current, and the first and second output signals may be currents based at least in part on the alternating current. In some instances, a first difference between the alternating current and the first output signal may be indicative of the presence or absence of one or more analytes of the first group of analytes, and a second difference between the alternating current and the second output signal may be indicative of the presence or absence of one or more analytes of the second group of analytes.

In other implementations, the first output signal may indicate a change in impedance of the first carbon-based sensor caused by exposure to one or more analytes of the first group of analytes, and the second output signal may indicate a change in impedance of the second carbon-based sensor caused by exposure to one or more analytes of the second group of analytes. In some instances, a relatively small impedance change of a respective carbon-based sensor may indicate an absence of a corresponding group of analytes, and a relatively large impedance change of the respective carbon-based sensor may indicate a presence of the corresponding group of analytes.

In some other implementations, the sensing device may include an antenna configured to receive an electromagnetic signal from an external device, and the first and second output signals may be frequency responses of the 3D graphene-based sensing materials of the first and second carbon-based sensors, respectively, to the electromagnetic signal. For example, the frequency response of the 3D graphene-based sensing materials of the first carbon-based sensor may be indicative of the presence or absence of one or more analytes of the first group of analytes, and the frequency response of the 3D graphene-based sensing materials of the second carbon-based sensor may be indicative of the presence or absence of one or more analytes of the second group of analytes. In some aspects, the frequency responses may be based on resonant impedance spectroscopy (RIS) sensing.

In various implementations, at least one of the output signals may indicate an operating mode of the battery pack. In some implementations, the at least one output signal may indicate a normal mode based on an absence of the analytes of the first group of analytes, may indicate a maintenance mode based on the presence of one or more analytes of the first group of analytes not exceeding a threshold level, or may indicate an emergency mode based on the presence of one or more analytes of the first group of analytes exceeding a threshold level. In addition, or in the alternative, the first output signal may be indicative of a concentration level of one or more analytes of the first group of analytes, and the second output signal may be indicative of a concentration level of one or more analytes of the second group of analytes.

In some implementations, the analytes of the first and second groups of analytes may include one or more volatile organic compounds (VOCs). The one or more volatile organic compounds (VOCs) include any one or more of carbon dioxide (CO₂), carbon monoxide (CO), nitrogen dioxide (NO₂), one or more hydrocarbons including methane (CH₄), ethylene (C₂H₄), ethane (C₂H₆), or propane (C₃H₈), one or more acids including hydrochloric acid (HCl) or hydrofluoric acid (HF), one or more fluorinated hydrocarbons including phosphorus oxyfluoride, hydrogen cyanide (HCN), one or more aromatics including benzene (C₆H₆), toluene (C₇H₈), ethanol (C₂H₅OH), hydrogen, carbonate based electrolytes including ethylene carbonate (C₃H₄O₃), dimethyl carbonate (C₃H₆O₃), propylene carbonate (C₄H₃O₃), or one or more reduced sulfur compounds including thiols having a form of R—SH. In some aspects, each of the 3D graphene-based sensing materials may be configured to adsorb the VOCs. In some aspects, each of the carbon-based sensors may include a plurality of different graphene allotropes. The plurality of different graphene allotropes of a respective carbon-based sensor may include one or more microporous pathways or mesoporous pathways.

Another innovative aspect of the subject matter described in this disclosure may be implemented as a container for storing one or more items. The container may include a surface defining a volume of the container and a label printed on the container. In various implementations, the label may include a substrate, a plurality of carbon-based sensors printed on the substrate, and one or more electrodes printed on the substrate. The carbon-based sensors may be collectively configured to detect a presence of one or more analytes within the container. In some implementations, each of the carbon-based sensors may be configured to react with a unique group of analytes in response to an electromagnetic signal received from an external device. The one or more electrodes may be coupled to at least some of the carbon-based sensors, and may be configured to provide one or more output signals indicating the presence or absence of the one or more analytes within the container. In some implementations, a first electrode coupled to the first carbon-based sensor may be configured to indicate the presence of one or more analytes of the first group of analytes, and a second electrode coupled to the second carbon-based sensor may be configured to confirm the presence of the analytes detected by the first carbon-based sensor. In some aspects, the carbon-based sensors may be configured to resonate at different frequencies in response to the electromagnetic signal.

In some implementations, a first carbon-based sensor may be functionalized with a first material configured to detect the presence of each analyte of a first group of analytes, and a second carbon-based sensor may be functionalized with a second material configured to detect the presence of each analyte of a second group of analytes, where the second group of analytes may be a subset of the first group of analytes. In some aspects, the first group of analytes may include at least twice as many different analytes as the second group of analytes. The second material may be different than the first material. For example, in one implementation, the first material may be cobalt-decorated carbon nano-onions (CNOs) configured to detect the presence of one or more of triacetone triperoxide (TATP), toluene, ammonia, or hydrogen sulfide (H₂S), and the second material may be iron-decorated three-dimensional (3D) graphene-inclusive structures configured to confirm the presence of toluene. For another example, a third carbon-based sensor may be functionalized with a third material configured to detect the presence of each analyte of a third group of analytes, where the third group of analytes is another subset of the first group of analytes, and the third material is different than the first and second materials.

In some implementations, each output signal may indicate a frequency response of a corresponding carbon-based sensor to the electromagnetic signal. In some instances, a first frequency response of the first carbon-based sensor to the electromagnetic signal may be indicative of the presence or absence of the analytes of the first group of analytes within the container, and a second frequency response of the second carbon-based sensor to the electromagnetic signal may be indicative of the presence or absence of the analytes of the second group of analytes within the container. The first frequency response may be based at least in part on exposure of the first carbon-based sensor to the electromagnetic signal for a first period of time, and the second frequency response may be based at least in part on exposure of the second carbon-based sensor to the electromagnetic signal for a second period of time that is longer than the first period of time. In some aspects, the second period of time is at least twice as long as the first period of time. The first and second frequency responses may be based on resonant impedance spectroscopy (RIS) sensing.

In various implementations, an antenna may be printed on the substrate and configured to drive a current through the carbon-based sensors in response to the electromagnetic signal. In some aspects, each output signal may indicate an impedance or reactance of a corresponding carbon-based sensor to the current. The impedance or reactance of the carbon-based sensors may be indicative of the presence or absence of the one or more analytes within the container. For example, the impedance or reactance of the first carbon-based sensor may be indicative of the presence or absence of an analyte of the first group of analytes, and the impedance or reactance of the second carbon-based sensor may be indicative of the presence or absence of an analyte of the second group of analytes. In some instances, at least two of the carbon-based sensors are juxtaposed in a planar arrangement on the substrate. In other instances, the carbon-based sensors are stacked on top of one another. In some aspects, the carbon-based sensors may form a permittivity gradient.

In some implementations, each of the carbon-based sensing materials may include a plurality of different graphene allotropes. In some aspects, the different graphene allotropes of a respective carbon-based sensor may include one or more microporous pathways or mesoporous pathways. Each of the carbon-based sensors may include a polymer configured to bind the plurality of different graphene allotropes to one another. The polymer may include humectants configured reduce a susceptibility of a respective carbon-based sensor to humidity.

In some implementations, a sensing device for detecting analytes within a package or container is disclosed herein. In various implementations, the sensing device may include a substrate, one or more electrodes, and a sensor array. The sensor array may be disposed on the substrate, and may include a plurality of carbon-based sensors coupled to the one or more electrodes. The carbon-based sensors may be configured to react with unique groups of analytes in response to an electromagnetic signal received from an external device. In some instances, a first sensor may be configured to detect a presence of each analyte of a group of analytes, and a second sensor may be configured to confirm the presence of each analyte of a subset of the group of analytes.

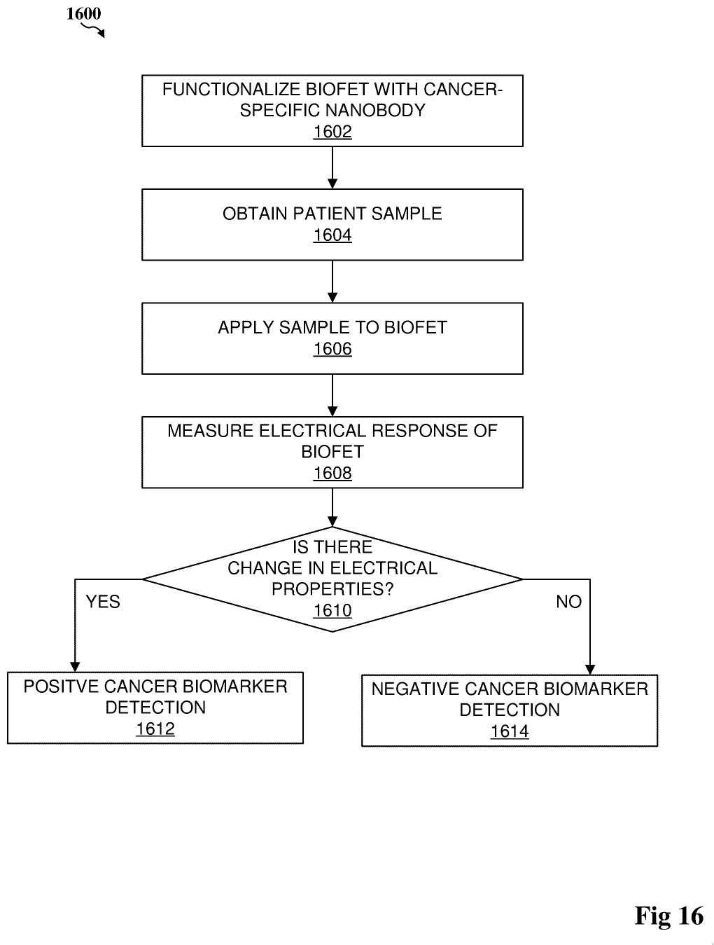

FIG. 16 illustrates a flowchart of a method 1600 for biomarker detection using functionalized bioFETs, in accordance with one embodiment. As an option, the method 1600 may be implemented in the context of any one or more of the embodiments set forth in any previous and/or subsequent Figures and/or description thereof. Of course, however, the method 1600 may be implemented in the context of any desired environment. Further, the aforementioned definitions may equally apply to the description below.

The method 1600 begins with a step 1602 of functionalizing a bioFET with a cancer-specific nanobody. For example, the bioFET may be prepared by attaching cancer-specific nanobodies to the surface of a graphene-containing field effect transistor (GFET). This functionalization process may involve covalent or non-covalent binding of the nanobodies to the graphene surface, creating a biosensor capable of selectively detecting cancer biomarkers. The selection of appropriate nanobodies may be based on their affinity and specificity for particular cancer biomarkers. In some cases, the functionalization process may include a surface treatment step to enhance nanobody attachment and orientation on the graphene surface.

In various embodiments, the functionalization step may be customized for different types of cancer biomarkers by selecting appropriate nanobodies. For example, nanobodies specific to HE4 may be used for ovarian cancer detection, while nanobodies targeting PSA may be employed for prostate cancer screening. This versatility allows the method 1600 to be adapted for various cancer types and other diseases. The nanobodies may be produced using recombinant DNA technology and may undergo rigorous screening processes to ensure high specificity and sensitivity for their target biomarkers. In some implementations, a combination of different nanobodies may be used to create a multiplexed detection system for multiple cancer biomarkers simultaneously.

In step 1604, a patient sample is obtained. This step may involve collecting a biological specimen such as blood, urine, or saliva from a patient. The sample collection process may be performed using standard medical procedures. The choice of sample type may depend on the specific cancer biomarkers being targeted and their known distribution in different bodily fluids. In some cases, multiple sample types may be collected from the same patient to increase the likelihood of detecting cancer biomarkers.

The patient sample obtained may be subjected to pre-processing steps such as centrifugation, filtration, or dilution to prepare the sample for application to the bioFET. These pre-processing steps may help remove potential interferents and optimize the sample for biomarker detection. The specific pre-processing protocols may be tailored to the type of sample and the target biomarkers. In some implementations, automated sample preparation systems may be used to standardize the pre-processing steps and minimize variability between samples.

In a step 1606, the sample is applied to the bioFET. For example, the prepared patient sample may be introduced to the functionalized bioFET surface, allowing any cancer biomarkers present in the sample to interact with the nanobodies on the bioFET. The sample application process may be optimized to ensure sufficient contact time between the sample and the bioFET surface while minimizing non-specific binding. In some cases, gentle agitation or flow-based systems may be used to enhance the interaction between biomarkers and nanobodies.

Additionally, the applying of the sample may be performed using microfluidic channels or droplet deposition techniques to ensure precise and controlled delivery of the sample to the bioFET surface. This controlled application helps maintain the sensitivity and reproducibility of the biomarker detection process. The microfluidic channels may be designed with specific geometries to optimize sample flow and minimize sample volume requirements. In some implementations, the sample application system may include integrated washing steps to remove unbound molecules and reduce background noise.

At a step 1608, the method 1600 involves measuring the electrical response of the bioFET. For example, changes in the electrical properties of the bioFET resulting from biomarker binding may be monitored and recorded. These changes may include alterations in drain current, conductance, or other electrical parameters of the bioFET. The measurement process may involve multiple time points to capture the kinetics of biomarker binding and to distinguish between specific and non-specific interactions. In some cases, differential measurements between functionalized and non-functionalized bioFETs may be used to further enhance the signal-to-noise ratio.

The measurement step 1608 may involve applying a voltage sweep across the bioFET and recording the resulting current-voltage characteristics. Other measurement techniques such as lock-in amplification or noise reduction algorithms may be employed to enhance the sensitivity and accuracy of the electrical measurements. The voltage sweep parameters may be optimized for different biomarkers and sample types to maximize detection sensitivity. In some implementations, multiple measurement modalities, such as impedance spectroscopy or pulsed voltage techniques, may be used in combination to provide complementary information about biomarker binding events.

The method 1600 then moves to a decision point at a decision step 1610, where a determination is made whether there is a change in electrical properties. For example, the measured electrical response may be compared to baseline values or predetermined thresholds to assess whether significant changes indicative of biomarker binding have occurred. The threshold values may be dynamically adjusted based on factors such as sample type, patient demographics, or known interfering substances. In some cases, the decision-making process may incorporate historical data from previous measurements to account for device-to-device variability and long-term drift.

The decision-making process in decision step 1610 may incorporate statistical analysis and machine learning algorithms to accurately interpret the electrical measurements and distinguish between true positive signals and background noise. This advanced data processing may help improve the reliability and specificity of the biomarker detection method. The machine learning models may be trained on large datasets of known positive and negative samples to improve their accuracy over time. In some implementations, the decision-making process may also consider the kinetics of the electrical response to further differentiate between specific and non-specific binding events.

If there is a change in electrical properties (Y es branch from decision step 1610), the method 1600 proceeds to a step 1612, where a positive cancer biomarker detection is indicated. For example, the system may generate an alert or report indicating the presence of cancer biomarkers in the patient sample. The alert system may be designed to provide clear and actionable information to healthcare providers, potentially including confidence levels or risk scores associated with the positive detection. In some cases, the system may also suggest follow-up tests or provide guidance on interpreting the results in the context of other clinical information.

The positive detection result in step 1612 may trigger additional diagnostic procedures or recommend further medical evaluation. For example, the method 1600 may also provide quantitative information about the detected biomarkers, potentially aiding in cancer staging or treatment monitoring. The quantitative analysis may include estimations of biomarker concentration based on calibration curves or machine learning models. In some implementations, the system may track biomarker levels over time, providing valuable information for monitoring treatment response or disease progression.

If there is no change in electrical properties (No branch from decision step 1610), the method 1600 proceeds to a step 1614, where a negative cancer biomarker detection is indicated. For example, the system may generate a report stating that no cancer biomarkers were detected in the patient sample at levels above the detection threshold. The negative report may include information about the specific biomarkers tested and the sensitivity limits of the assay. In some cases, the system may recommend retesting or alternative screening methods based on the patient's risk factors or clinical history.

The negative detection result in step 1614 may be used to inform clinical decision-making, such as recommending routine follow-up screening or considering alternative diagnostic approaches. For example, the method 1600 may also provide information about the detection limits and sensitivity of the assay to aid in result interpretation. This information may help healthcare providers assess the reliability of the negative result and determine appropriate follow-up actions. In some implementations, the system may integrate the negative result with other clinical data to provide a comprehensive risk assessment for the patient.

In various embodiments, the method 1600 may be expanded to include multiple bioFETs functionalized with different cancer-specific nanobodies, allowing for simultaneous detection of multiple cancer biomarkers. This multi-analyte detection capability may enhance the diagnostic power of the method and provide a more comprehensive cancer risk assessment. The multi-bioFET array may be designed to detect complementary biomarkers that, when combined, offer improved diagnostic accuracy compared to single biomarker tests. In some cases, the array may include bioFETs functionalized with nanobodies targeting both cancer-specific and general inflammatory markers to provide a more complete picture of the patient's health status.

In various embodiments, the method 1600 may incorporate real-time monitoring of the bioFET electrical properties during sample application and incubation. This continuous measurement approach may provide kinetic information about biomarker binding, potentially offering insights into biomarker concentration or affinity. The real-time data may be used to generate binding curves that can be analyzed to determine association and dissociation rates of biomarkers. In some implementations, this kinetic information may be used to differentiate between biomarkers with similar structures but different binding kinetics, further enhancing the specificity of the detection method.

In various embodiments, the method 1600 may be integrated with a portable, field-deployable device that combines sample preparation, bioFET measurement, and data analysis in a single platform. This integrated system may enable rapid, on-site cancer biomarker detection in resource-limited settings or for point-of-care diagnostics. The portable device may include features such as a touch-screen interface for easy operation, built-in quality control measures, and wireless connectivity for data transmission and remote analysis. In some cases, the device may be designed with a modular architecture, allowing for easy upgrades or customization for different biomarker panels or disease targets.

The method 1600 exemplifies a resolution to challenges in traditional cancer biomarker detection methods, which often require complex laboratory equipment and time-consuming procedures. By utilizing functionalized bioFETs, the method 1600 offers a rapid, sensitive, and potentially portable approach to cancer biomarker detection that may be suitable for early cancer screening and monitoring. Furthermore, the method 1600 addresses limitations in conventional biosensing techniques by leveraging the unique properties of graphene-based FETs and the specificity of nanobodies. This combination may result in improved sensitivity and selectivity compared to traditional immunoassays, potentially enabling the detection of cancer biomarkers at earlier stages of disease progression.

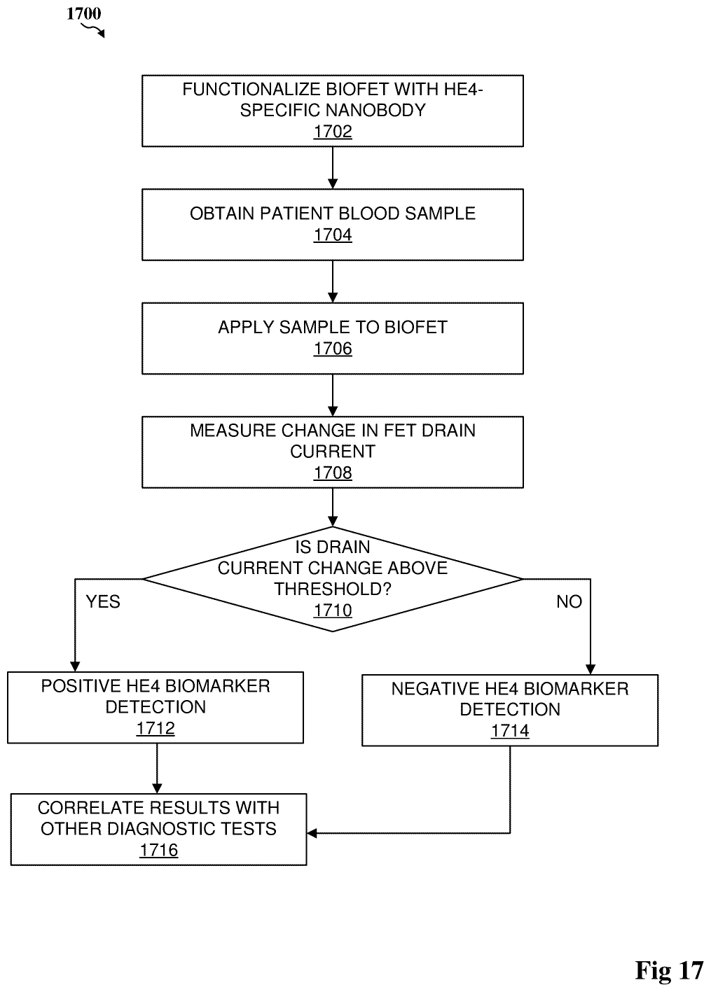

FIG. 17 illustrates a flowchart of a biomarker detection method 1700 using functionalized bioFETs, in accordance with one embodiment. As an option, the biomarker detection method 1700 may be implemented in the context of any one or more of the embodiments set forth in any previous and/or subsequent Figures and/or description thereof. Of course, however, the biomarker detection method 1700 may be implemented in the context of any desired environment. Further, the aforementioned definitions may equally apply to the description below.

The biomarker detection method 1700 begins with a step 1702 of functionalizing a bioFET with an HE4-specific nanobody. For example, a graphene-containing field effect transistor (GFET) may be modified by attaching nanobodies specifically designed to bind to Human Epididymis Protein 4 (HE4), a biomarker associated with ovarian cancer. This functionalization process may involve covalent or non-covalent attachment of the HE4-specific nanobodies to the graphene surface of the GFET. Such functionalization may also be consistent with step 1602 discussed hereinabove.

At a step 1704, the biomarker detection method 1700 involves obtaining a patient blood sample. In operation 1704, a blood specimen may be collected from a patient using standard phlebotomy techniques. The blood sample may be processed to separate serum or plasma, which typically contains the HE4 biomarker if present. The choice between serum and plasma may depend on factors such as the stability of HE4 in different blood fractions and the presence of potential interfering substances. In some cases, multiple aliquots of the blood sample may be prepared to allow for replicate measurements or additional testing if needed. The step 1704 may be also be consistent with the step 1704 discussed hereinabove.

In various embodiments, the sample collection step 1704 may include measures to preserve the integrity of potential biomarkers in the blood. These measures may include using appropriate anticoagulants, maintaining proper temperature during transport, and minimizing the time between collection and analysis to prevent degradation of the HE4 protein. The selection of anticoagulants may be based on their compatibility with HE4 and other potential biomarkers of interest. In some implementations, stabilizing agents or protease inhibitors may be added to the blood sample immediately after collection to further protect HE4 from degradation during processing and storage.

The biomarker detection method 1700 then proceeds to a step 1706, where the sample is applied to the bioFET. For example, the prepared blood sample or its derivatives (serum or plasma) may be introduced to the surface of the HE4-specific nanobody functionalized bioFET. This application may be performed using microfluidic channels or droplet deposition techniques to ensure uniform coverage and optimal interaction between the sample and the functionalized bioFET surface. The design of the microfluidic channels may incorporate features such as mixing chambers or serpentine paths to enhance the interaction between HE4 molecules and the functionalized surface. In some cases, the sample application system may include integrated filtration or purification steps to remove potential interfering substances before the sample reaches the bioFET surface.

The sample application step 1706 may involve careful control of factors such as sample volume, flow rate, and incubation time to maximize the chances of HE4 binding to the nanobodies while minimizing non-specific interactions. In some cases, blocking agents may be used to reduce background signals and improve the specificity of HE4 detection. The selection of blocking agents may be based on their effectiveness in preventing non-specific binding without interfering with the HE4-nanobody interaction. In some implementations, the sample application process may include multiple cycles of sample flow and washing steps to enhance the capture of low-abundance HE4 molecules while maintaining a low background signal.

At a step 1708, the biomarker detection method 1700 involves measuring a change in FET drain current. For example, the electrical characteristics of the bioFET may be monitored before, during, and after sample application. The binding of HE4 molecules to the nanobodies on the bioFET surface may cause changes in the local electric field, which in turn may affect the drain current of the FET. The measurement system may be designed to capture rapid changes in drain current, allowing for real-time monitoring of HE4 binding events. In some implementations, the bioFET may be integrated into an array format, enabling simultaneous measurements from multiple devices to improve reliability and throughput.

In various embodiments, the measurement step 1708 may employ other techniques such as lock-in amplification or differential measurements to enhance the signal-to-noise ratio and detect small changes in drain current. In some cases, multiple measurements may be taken over time to observe binding kinetics and improve the accuracy of HE4 quantification. The measurement protocol may include periodic reference measurements using control bioFETs or known HE4 concentrations to account for any drift in device performance over time. In some implementations, the measurement system may incorporate temperature control and compensation mechanisms to minimize the impact of thermal fluctuations on the electrical measurements.

The biomarker detection method 1700 then moves to a decision point at a decision step 1710, where a determination is made whether the drain current change is above a threshold. For example, the measured change in drain current may be compared to pre-established threshold values that indicate the presence of clinically significant levels of HE4. The threshold values may be determined through extensive clinical studies and may be adjusted based on factors such as patient age, menopausal status, or the presence of other medical conditions that could affect HE4 levels. In some cases, the decision-making process may incorporate dynamic thresholding techniques that adapt to variations in baseline measurements or environmental conditions.

The threshold determination decision step 1710 may incorporate calibration curves and statistical analysis to account for variations in bioFET performance and sample characteristics. Machine learning algorithms may be employed to improve the accuracy of threshold determination and reduce false positive or false negative results. The machine learning models may be trained on large datasets of HE 4 measurements from both healthy individuals and ovarian cancer patients, incorporating additional clinical and demographic data to improve classification accuracy. In some implementations, the decision-making process may include a confidence scoring system to indicate the reliability of the HE4 detection result based on factors such as signal strength, measurement stability, and agreement across multiple bioFET devices.

If the drain current change is above the threshold (Y es branch from decision step 1710), the biomarker detection method 1700 proceeds to a step 1712, where a positive HE4 biomarker detection is indicated. In various embodiments, the system may generate an alert or report suggesting the presence of elevated HE4 levels in the patient's blood sample, which may be indicative of ovarian cancer. The alert system may be designed to provide clear, actionable information to healthcare providers, potentially including visualizations of the HE4 levels relative to established clinical thresholds. In some implementations, the system may also provide an estimate of the likelihood of ovarian cancer based on the magnitude of HE4 elevation and other relevant patient factors.

The positive detection result in step 1712 may trigger recommendations for further diagnostic procedures, such as imaging studies or additional blood tests. The biomarker detection method 1700 may also provide quantitative information about the detected HE4 levels, potentially aiding in disease staging or treatment monitoring. The quantitative analysis may include an estimation of HE4 concentration based on calibration curves, as well as information about the measurement uncertainty and detection limits. In some cases, the system may also provide historical context if previous HE 4 measurements are available for the patient, allowing for the assessment of changes in HE4 levels over time.

If the drain current change is not above the threshold (No branch from decision step 1710), the biomarker detection method 1700 proceeds to a step 1714, where a negative HE4 biomarker detection is indicated. In various embodiments, the system may generate a report stating that HE4 levels in the patient's blood sample are below the detection threshold for ovarian cancer risk. The negative report may include information about the specific threshold used for the determination and any relevant caveats or limitations of the assay. In some implementations, the system may provide guidance on the interpretation of negative results in the context of the patient's overall risk profile for ovarian cancer.

The negative detection result in step 1714 may be used to inform clinical decision-making, such as recommending routine follow-up screening or considering alternative diagnostic approaches. The biomarker detection method 1700 may also provide information about the detection limits and sensitivity of the assay to aid in result interpretation. This information may include the lower limit of detection for HE4, as well as data on the assay's performance characteristics such as specificity and negative predictive value. In some cases, the system may suggest additional biomarker tests or screening methods that could complement the HE4 measurement, especially for patients with other risk factors for ovarian cancer.

Following either the positive or negative detection result, the biomarker detection method 1700 proceeds to a step 1716, where the results are correlated with other diagnostic tests. In operation 1716, the HE4 biomarker detection results may be integrated with other clinical data, such as CA 125 levels, ultrasound findings, or patient symptoms, to provide a more comprehensive assessment of ovarian cancer risk (and/or any other tested cancer risk). The integration process may involve standardized algorithms or risk calculation tools that combine multiple biomarker results with imaging findings and clinical factors. In some implementations, the system may interface with electronic health records to automatically retrieve relevant patient data and incorporate it into the risk assessment.

In various embodiments, the correlation step 1716 may involve sophisticated data analysis techniques, including multivariate statistical methods or artificial intelligence algorithms, to combine multiple biomarker results and clinical parameters. This integrated approach may improve the overall accuracy and clinical utility of the ovarian cancer screening process. The data analysis may include techniques such as logistic regression, decision trees, or neural networks to generate a composite risk score or probability estimate for ovarian cancer. In some cases, the system may provide visualizations or decision support tools to help clinicians interpret the combined results and make informed decisions about patient management or further diagnostic workup.

In various embodiments, the biomarker detection method 1700 may be adapted for the detection of biomarkers associated with other cancer types or diseases. By modifying the nanobody functionalization in step 1702, the method may be tailored to detect biomarkers for conditions such as prostate cancer (using PSA-specific nanobodies), breast cancer (using HER2-specific nanobodies), autoimmune diseases (using autoantibody-specific nanobodies), and/or other similar type associates. This versatility may allow for the development of a platform technology for rapid, sensitive biomarker detection across a wide range of medical applications.

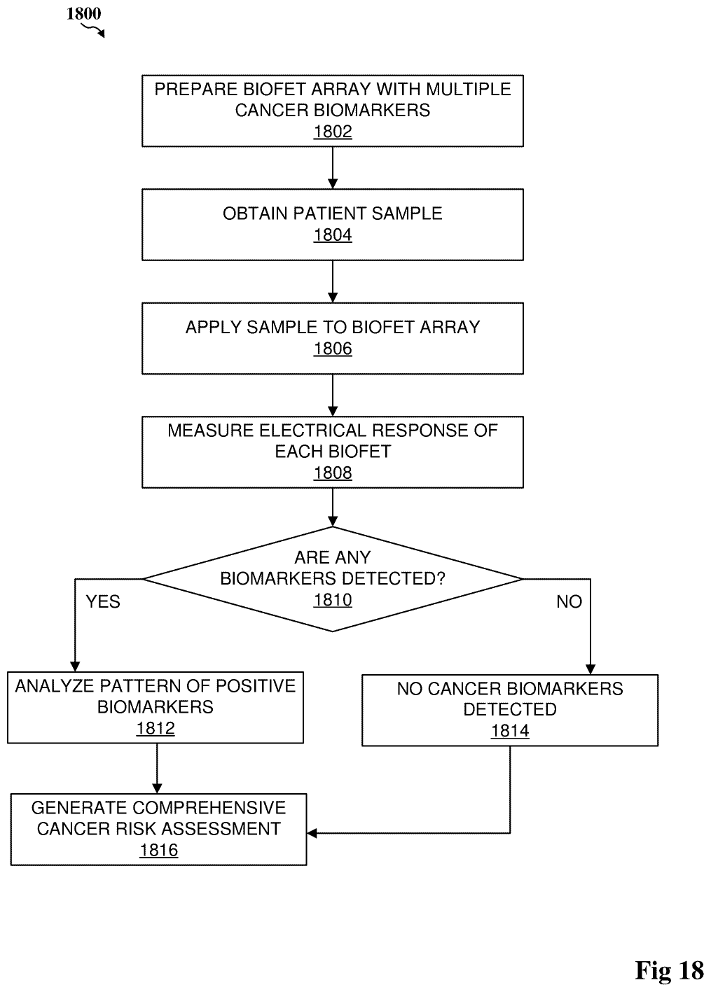

FIG. 18 illustrates a flowchart of a method 1800 for biomarker detection using functionalized bioFETs, in accordance with one embodiment. As an option, the method 1800 may be implemented in the context of any one or more of the embodiments set forth in any previous and/or subsequent Figures and/or description thereof. Of course, however, the method 1800 may be implemented in the context of any desired environment. Further, the aforementioned definitions may equally apply to the description below.

The method 1800 begins with a step 1802 of preparing a bioFET array with multiple cancer biomarkers. In various embodiments, a bioFET array may be fabricated using three-dimensional (3D) graphene structures to form graphene-containing field effect transistors (GFETs). The 3D graphene structure may be configured to increase the surface area for biomarker binding, thereby enhancing the sensitivity of the bioFETs. The fabrication process may involve techniques such as chemical vapor deposition or plasma-enhanced growth to create the 3D graphene structures directly on the sensor substrate. In some implementations, the 3D graphene may be further modified with additional functional groups or nanoparticles to enhance its electrical properties or biocompatibility.

In various embodiments, the preparation step may involve functionalizing each bioFET in the array with different cancer-specific nanobodies or aptamers. For example, one bioFET may be functionalized with HE4-specific nanobodies for ovarian cancer detection, while another may be functionalized with PSA-specific aptamers for prostate cancer screening. This multi-biomarker approach may allow for simultaneous detection of various cancer types or stages. The functionalization process may be optimized for each biomarker, considering factors such as binding affinity, orientation, and density of the recognition elements on the 3D graphene surface. In some cases, a combination of different recognition elements may be used on a single bioFET to create multi-modal sensing capabilities or to improve specificity through cooperative binding effects.

At a step 1804, the method 1800 involves obtaining a patient sample. In various embodiments, a biological specimen such as blood, urine, or saliva may be collected from a patient using standard medical procedures. The sample collection process may be tailored to preserve the integrity of potential biomarkers and minimize interference from other biological components. The choice of sample type may depend on the specific biomarkers being targeted and their known distribution in different bodily fluids. In some implementations, multiple sample types may be collected from the same patient to provide a more comprehensive biomarker profile or to cross-validate results across different sample matrices.

The method 1800 proceeds to a step 1806, where the sample is applied to the bioFET array. In various embodiments, the prepared patient sample may be introduced to the functionalized bioFET array using microfluidic channels or automated dispensing systems. This step may ensure uniform distribution of the sample across all bioFET sensors in the array. The microfluidic system may be designed with specific channel geometries or flow patterns to optimize sample-sensor contact and minimize sample volume requirements. In some implementations, the sample application process may include integrated quality control measures, such as real-time monitoring of flow rates or pressure, to ensure consistent and reliable sample delivery across multiple tests.

In various embodiments, the sample application step may be performed under controlled conditions, such as maintaining a specific temperature or pH, to optimize biomarker-nanobody interactions. In some cases, the sample may be incubated on the bioFET array for a predetermined time to allow for sufficient binding of biomarkers to their respective functionalized surfaces. The incubation conditions may be dynamically adjusted based on real-time monitoring of binding kinetics, potentially using initial rapid measurements to estimate optimal incubation times for each biomarker. In some implementations, the sample application system may include integrated washing steps or buffer exchanges to remove unbound molecules and enhance signal specificity.

At a step 1808, the method 1800 measures the electrical response of each bioFET in the array. For example, changes in electrical properties such as drain current, conductance, or threshold voltage may be monitored and recorded for each bioFET sensor. These measurements may be performed simultaneously across all sensors in the array to provide a comprehensive biomarker profile. The measurement system may employ multiplexing techniques to efficiently capture data from multiple sensors in parallel, potentially using dedicated readout circuits for each bioFET to maximize sensitivity. In some cases, the electrical measurements may be performed at multiple time points or under varying bias conditions to capture dynamic binding information or to probe different aspects of the sensor-analyte interaction.

In various embodiments, the measurement step 1808 may employ advanced signal processing techniques to enhance the sensitivity and specificity of biomarker detection. For example, differential measurements between functionalized bioFETs and control bioFETs may be used to account for non-specific binding and improve the signal-to-noise ratio. The signal processing algorithms may incorporate adaptive filtering or noise cancellation techniques to compensate for environmental fluctuations or device-to-device variations. In some implementations, the measurement system may include self-calibration features that periodically assess sensor performance and adjust measurement parameters to maintain optimal sensitivity throughout the testing process.

The method 1800 then moves to a decision point at a decision step 1810, where a determination is made if any biomarkers are detected. For example, the measured electrical responses from each bioFET in the array may be compared to pre-established thresholds or calibration curves to determine the presence and concentration of specific cancer biomarkers. The decision-making process may incorporate statistical analysis techniques such as hypothesis testing or Bayesian inference to assess the significance of observed signal changes. In some cases, the detection thresholds may be dynamically adjusted based on the overall signal profile of the sample or patient-specific factors to optimize sensitivity and specificity for each individual test.

The decision-making process in decision step 1810 may incorporate machine learning algorithms trained on large datasets of cancer biomarker profiles. These algorithms may help interpret complex patterns of biomarker expression and improve the accuracy of cancer detection and classification. The machine learning models may be designed to handle multi-dimensional data, considering not only the absolute levels of individual biomarkers but also their relative ratios and temporal changes. In some implementations, the algorithms may incorporate transfer learning techniques to adapt pre-trained models to specific patient populations or clinical contexts, potentially improving performance in diverse healthcare settings.

If biomarkers are detected (Y es branch from decision step 1810), the method 1800 proceeds to a step 1812, where the pattern of positive biomarkers is analyzed. For example, the system may evaluate the combination and levels of detected biomarkers to assess the likelihood of specific cancer types or stages. The analysis may employ pattern recognition algorithms or decision tree models to classify the observed biomarker profile into predefined cancer categories. In some cases, the analysis may also consider the kinetics of biomarker binding or the relative changes in biomarker levels compared to previous measurements to provide insights into disease progression or treatment response.

In various embodiments, the analysis in step 1812 may consider the relative expression levels of different biomarkers and their known associations with various cancer types. For example, elevated levels of both HE4 and CA 125 may strongly indicate ovarian cancer, while a combination of PSA and PCA 3 may suggest prostate cancer. The analysis may also take into account the specificity and sensitivity of each biomarker, potentially weighting their contributions to the overall assessment based on their individual diagnostic performance. In some implementations, the system may use probabilistic models to generate likelihood ratios for different cancer types, providing clinicians with a quantitative basis for interpreting the multi-biomarker results.

If no biomarkers are detected (No branch from decision step 1810), the method 1800 moves to a step 1814, where it is noted that no cancer biomarkers are detected. For example, the system may generate a report indicating that the measured biomarker levels are below the detection thresholds for the cancer types being screened. The report may include detailed information about the sensitivity and specificity of the assay for each biomarker, helping clinicians interpret the significance of the negative result. In some cases, the system may perform additional quality control checks or suggest repeat testing to confirm the negative result, especially for high-risk patients or in cases where the measured signals are close to the detection threshold.

The negative result in step 1814 may be accompanied by information about the detection limits of the assay and recommendations for follow-up testing or monitoring. In some cases, the absence of detected biomarkers may be used to rule out certain cancer types with high confidence. The system may provide guidance on appropriate intervals for repeat screening based on the patient's risk factors and the performance characteristics of the bioFET array. In some implementations, the negative result may trigger suggestions for alternative screening methods or lifestyle interventions to maintain low cancer risk, tailored to the individual patient's health profile.

Following either step 1812 or step 1814, the method 1800 proceeds to a step 1816, where a comprehensive cancer risk assessment is generated based on the analysis of detected biomarkers or lack thereof. For example, the system may integrate the biomarker detection results with other clinical data, such as patient history, genetic risk factors, or imaging results, to provide a holistic assessment of cancer risk. The integration process may involve other data fusion algorithms that may normalize and combine diverse data types, potentially using ontology-based approaches to ensure semantic consistency across different information sources. In some cases, the system may access external databases or knowledge bases to incorporate the latest research findings or population-level statistics into the risk assessment.

In various embodiments, the comprehensive risk assessment generated in step 1816 may use other statistical models or artificial intelligence algorithms to weigh the significance of different biomarkers and clinical factors. This assessment may provide clinicians with actionable insights for patient management, such as recommendations for additional diagnostic tests or personalized screening schedules. The risk assessment models may be continuously updated based on feedback from clinical outcomes, potentially employing federated learning techniques to improve performance across multiple healthcare institutions while maintaining patient privacy. In some implementations, the system may generate personalized visualizations or risk communication tools to help patients understand their cancer risk profile and engage in shared decision-making with their healthcare providers.

It is to be appreciated that the method 1800 is an improvement over traditional cancer screening approaches, which often rely on single biomarker tests or invasive procedures. By utilizing a multi-biomarker bioFET array, the method 1800 offers a comprehensive, rapid, and potentially non-invasive approach to cancer risk assessment that may improve early detection rates and reduce false positive results. Furthermore, the method 1800 addresses limitations in conventional biomarker detection methods by leveraging the unique properties of 3D graphene-based FETs. The increased surface area provided by the 3D graphene structure may enhance biomarker binding and improve sensitivity, potentially enabling the detection of cancer biomarkers at earlier stages or lower concentrations than traditional assays.

In various embodiments, the method 1800 may be adapted for the detection of biomarkers associated with other diseases beyond cancer. For example, by modifying the functionalization of the bioFET array, the method may be applied to screening for cardiovascular diseases, neurodegenerative disorders, or autoimmune conditions. This versatility may allow for the development of a comprehensive health screening platform using a single, portable bioFET-based device.

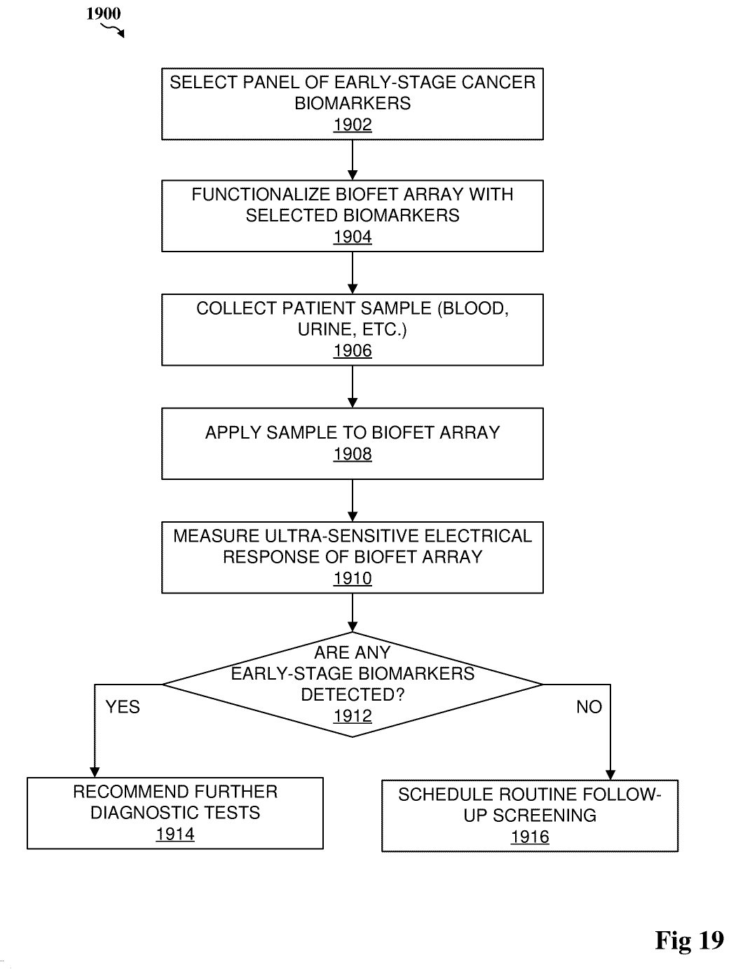

FIG. 19 illustrates a flowchart of a method 1900 for early-stage cancer biomarker detection, in accordance with one embodiment. As an option, the method 1900 may be implemented in the context of any one or more of the embodiments set forth in any previous and/or subsequent Figures and/or description thereof. Of course, however, the method 1900 may be implemented in the context of any desired environment. Further, the aforementioned definitions may equally apply to the description below.

The method 1900 begins at a step 1902 with selecting a panel of early-stage cancer biomarkers. In various embodiments, a set of biomarkers known to be associated with early stages of various cancer types may be chosen for detection. This selection may be based on extensive research and clinical data identifying molecules that appear in bodily fluids at the earliest stages of cancer development. The biomarker panel may be customized for different cancer types or patient populations, taking into account factors such as genetic predisposition and environmental risk factors. In some cases, the selection process may involve a combination of literature review, experimental validation, and consultation with oncology experts to ensure a comprehensive and up-to-date panel of early-stage biomarkers.

In various embodiments, the biomarker selection in step 1902 may include proteins, nucleic acids, metabolites, or other biological molecules that show altered expression or presence in early-stage cancers. For example, the panel may include biomarkers such as circulating tumor DNA (ctDNA), microRNAs, or specific proteins that are overexpressed in cancer cells before clinical symptoms appear. The selection may also consider emerging biomarkers identified through advanced genomic and proteomic studies, potentially incorporating novel molecules with high specificity for early-stage cancer detection. In some implementations, the panel may include a combination of tissue-specific and pan-cancer biomarkers to provide both targeted and broad-spectrum early detection capabilities.

At a step 1904, the method 1900 proceeds to functionalize a bioFET array with the selected biomarkers. In various embodiments, the surface of each bioFET in the array may be modified to specifically bind to one of the selected early-stage cancer biomarkers. This functionalization process may involve attaching antibodies, aptamers, or other recognition elements that can selectively capture the target biomarkers. The choice of recognition element may be tailored to the specific properties of each biomarker, considering factors such as size, charge, and potential for non-specific interactions. In some cases, the functionalization process may involve multiple steps, including surface activation, linker attachment, and recognition element coupling, each optimized for maximum binding efficiency and stability.

In various embodiments, the functionalization in step 1904 may be optimized to ensure high specificity and sensitivity for each biomarker. Techniques such as covalent binding, self-assembled monolayers, or click chemistry may be employed to create a stable and uniform layer of recognition elements on the bioFET surface. The density and orientation of the recognition elements may be carefully controlled to maximize binding capacity while minimizing steric hindrance. In some implementations, the functionalization process may include the use of spacer molecules or hydrogel matrices to enhance the accessibility of the recognition elements to the target biomarkers in complex biological samples.

The method 1900 continues at a step 1906 with collecting a patient sample. In various embodiments, a biological specimen such as blood, urine, saliva, or cerebrospinal fluid may be obtained from the patient. The choice of sample type may depend on the specific biomarkers being targeted and their known distribution in bodily fluids. The sample collection process may be optimized for each biofluid, considering factors such as circadian variations in biomarker levels and potential confounding factors like diet or medication. In some cases, multiple sample types may be collected from the same patient to provide a more comprehensive biomarker profile and increase the likelihood of detecting early-stage cancer.

In various embodiments, the sample collection in step 1906 may involve standardized procedures to ensure the integrity of potential biomarkers. This may include using specific collection tubes, maintaining appropriate temperature conditions, and minimizing the time between collection and analysis to prevent degradation of sensitive biomarkers. The collection protocol may also include steps to minimize cellular contamination or activation, which could affect the levels of certain biomarkers. In some implementations, the sample collection process may incorporate point-of-care stabilization techniques, such as the addition of preservatives or immediate processing, to maintain the integrity of labile biomarkers during transport and storage.

At a step 1908, the collected sample is applied to the bioFET array. In operation 1908, the patient sample may be introduced to the functionalized bioFET array using microfluidic channels or automated dispensing systems. This step may ensure uniform distribution of the sample across all bioFET sensors in the array. The sample application process may be optimized to maximize the interaction between biomarkers and their corresponding recognition elements, potentially incorporating techniques such as recirculation or pulsed flow to enhance capture efficiency. In some cases, the sample application system may include integrated mixing or dilution capabilities to accommodate variations in sample viscosity or biomarker concentration.

Additionally, the sample application in step 1908 may be performed under controlled conditions, such as maintaining a specific temperature or pH, to optimize biomarker-recognition element interactions. In some cases, the sample may be pre-processed to remove interfering substances or concentrate the biomarkers of interest before application to the bioFET array. The pre-processing steps may include techniques such as size exclusion filtration, affinity purification, or exosome isolation, depending on the nature of the target biomarkers. In some implementations, the sample application process may incorporate real-time monitoring of environmental parameters and automated adjustment mechanisms to maintain optimal conditions throughout the analysis.

At a step 1910, the method 1900 involves measuring ultra-sensitive electrical response of the bioFET array. For example, changes in electrical properties such as drain current, conductance, or threshold voltage may be monitored and recorded for each bioFET sensor in the array. These measurements may be performed with high precision to detect even minute changes caused by biomarker binding. The measurement system may employ advanced electronic components, such as low-noise amplifiers and high-resolution analog-to-digital converters, to capture subtle changes in electrical signals. In some cases, the measurement process may include multiple sampling points or extended integration times to improve signal quality and reduce random fluctuations.

In various embodiments, the ultra-sensitive measurement in step 1910 may employ advanced signal processing techniques and noise reduction algorithms to enhance the detection of low-abundance biomarkers. For example, lock-in amplification or differential measurement strategies may be used to improve the signal-to-noise ratio and enable detection of biomarkers at femtomolar concentrations. The signal processing algorithms may incorporate adaptive filtering techniques to compensate for drift or environmental variations during the measurement period. In some implementations, the measurement system may utilize parallel processing or field-programmable gate arrays (FPGAs) to perform real-time signal analysis and feature extraction, potentially enabling rapid detection of significant binding events.

The method 1900 then moves to a decision point at a decision step 1912, where it is determined whether any early-stage biomarkers are detected. For example, the measured electrical responses from each bioFET in the array may be compared to pre-established thresholds or calibration curves to determine the presence and concentration of specific early-stage cancer biomarkers. The decision-making process may incorporate statistical techniques such as hypothesis testing or Bayesian inference to assess the significance of observed signal changes. In some cases, the detection thresholds may be dynamically adjusted based on the overall signal profile of the sample or patient-specific factors to optimize sensitivity and specificity for each individual test.

In various embodiments, the decision-making process in step 1912 may incorporate machine learning algorithms trained on large datasets of early-stage cancer biomarker profiles. These algorithms may help interpret complex patterns of biomarker expression and improve the accuracy of early cancer detection, even when individual biomarker levels are below traditional detection limits. The machine learning models may be designed to handle multi-dimensional data, considering not only the absolute levels of individual biomarkers but also their relative ratios and temporal changes. In some implementations, the algorithms may incorporate transfer learning techniques to adapt pre-trained models to specific patient populations or clinical contexts, potentially improving performance in diverse healthcare settings.

Following the decision at decision step 1912, the method 1900 branches into two paths. If early-stage biomarkers are detected (YES branch), the method 1900 proceeds to a step 1914, where further diagnostic tests are recommended. For example, the system may generate a report suggesting additional, more specific diagnostic procedures based on the detected biomarkers. The recommendations may be prioritized based on the likelihood and potential severity of the indicated cancer type, taking into account factors such as the patient's age, medical history, and risk factors. In some cases, the system may provide a detailed explanation of the biomarker results and their implications, including visualizations or comparative data to help healthcare providers interpret the findings.

In various embodiments, the recommendations in step 1914 may be tailored to the specific biomarkers detected and their associated cancer types. For example, detection of certain ctDNA markers may prompt recommendations for advanced imaging studies or tissue biopsies to confirm the presence of early-stage cancer. The system may also suggest specific molecular or genetic tests to further characterize the potential cancer, such as next-generation sequencing or immunohistochemistry. In some implementations, the recommendations may include options for minimally invasive follow-up tests or emerging diagnostic technologies that are particularly suited for early-stage cancer detection.

If no early-stage biomarkers are detected (NO branch), the method 1900 proceeds to a step 1916, where routine follow-up screening is scheduled. For example, the system may generate a report indicating that no early-stage cancer biomarkers were detected at levels above the ultra-sensitive detection thresholds. The report may include detailed information about the sensitivity and specificity of the assay for each biomarker, helping clinicians interpret the significance of the negative result. In some cases, the system may perform additional quality control checks or suggest repeat testing to confirm the negative result, especially for high-risk patients or in cases where the measured signals are close to the detection threshold.

In various embodiments, the follow-up scheduling in step 1916 may be personalized based on the individual's risk factors and the sensitivity of the bioFET array. For example, individuals with higher risk profiles may be recommended for more frequent follow-up screenings, even in the absence of detected biomarkers. The scheduling algorithm may take into account factors such as family history, genetic predisposition, and lifestyle factors to determine the optimal screening interval. In some implementations, the system may provide educational materials or lifestyle recommendations to help patients maintain their health and reduce cancer risk between screenings, potentially incorporating personalized prevention strategies based on the individual's risk profile and biomarker results.

Taking a step back, the method 1900 shows an improvement to traditional cancer screening approaches, which often lack sensitivity for detecting cancer at its earliest, most treatable stages. By utilizing an ultra-sensitive bioFET array capable of detecting biomarkers at femtomolar concentrations, the method 1900 offers a potential breakthrough in early cancer detection that may significantly improve patient outcomes through earlier intervention. Additionally, the method 1900 addresses limitations in conventional biomarker detection methods by leveraging the unique properties of functionalized bioFETs. The ability to detect biomarkers such as ricin, cortisol, and fentanyl at concentrations as low as 1-10 femtomolar demonstrates the exceptional sensitivity of the system, which may be used for identifying the subtle molecular changes associated with early-stage cancer.

It is acknowledged that the method 1900 may be expanded to include a larger panel of early-stage biomarkers, potentially covering a wider range of cancer types or subtypes. This expanded panel may improve the overall sensitivity and specificity of the early cancer detection process, reducing false positives and negatives. In various embodiments, the method 1900 may be adapted for the detection of early-stage biomarkers associated with other diseases beyond cancer. By modifying the functionalization of the bioFET array, the method may be applied to screening for early stages of neurodegenerative disorders, autoimmune conditions, or infectious diseases. This versatility may allow for the development of a comprehensive early disease detection platform using a single, ultra-sensitive bioFET-based device.

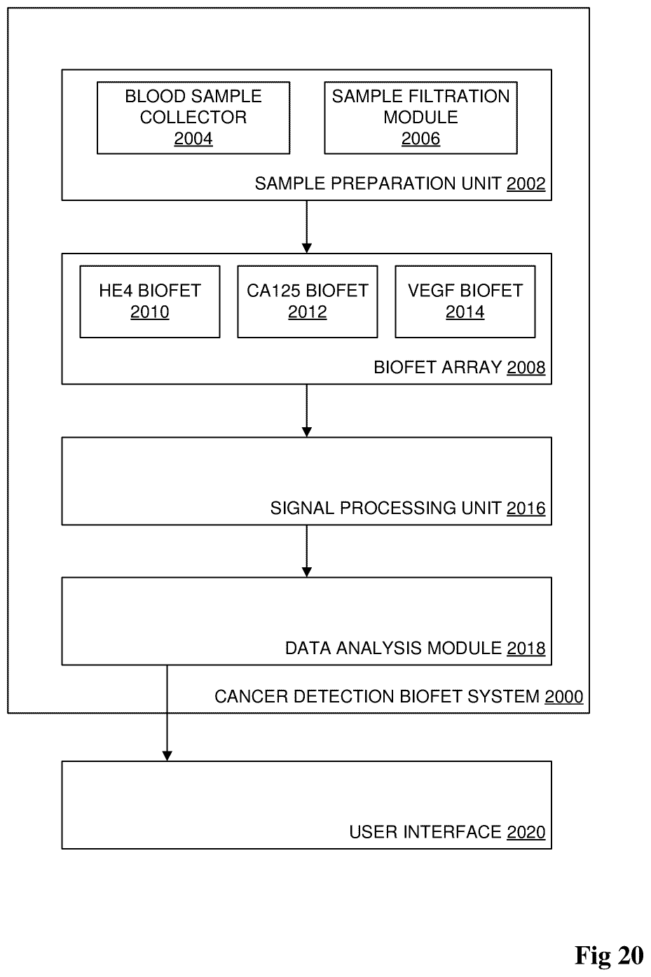

FIG. 20 illustrates a cancer detection system 2000, in accordance with one embodiment. As an option, the cancer detection system 2000 may be implemented in the context of any one or more of the embodiments set forth in any previous and/or subsequent Figures and/or description thereof. Of course, however, the cancer detection system 2000 may be implemented in the context of any desired environment. Further, the aforementioned definitions may equally apply to the description below.

The cancer detection system 2000 includes a sample preparation unit 2002 that comprises a blood sample collector 2004 and a sample filtration module 2006. In various embodiments, the blood sample collector 2004 may receive blood samples from patients for cancer biomarker analysis. The blood sample collector 2004 may utilize standardized blood collection techniques to ensure sample integrity and consistency. The blood sample collector 2004 may incorporate automated sample labeling and tracking systems to minimize human error and ensure proper sample identification throughout the analysis process. Additionally, the collector may be equipped with temperature-controlled storage compartments to maintain optimal conditions for different biomarker types immediately after collection.

The sample preparation unit 2002 may incorporate measures to preserve the stability of potential cancer biomarkers in the collected blood samples. These measures may include using appropriate anticoagulants, maintaining proper temperature during sample handling, and minimizing the time between collection and analysis to prevent degradation of sensitive biomarkers. The unit may also employ specialized sample preservation buffers that contain protease inhibitors and stabilizing agents tailored to specific biomarker classes. Furthermore, the sample preparation unit 2002 may utilize rapid cooling techniques, such as flash freezing, for samples that require long-term storage before analysis.

Additionally, the sample filtration module 2006 may process the collected blood samples to remove interfering substances and optimize the samples for biomarker detection. The sample filtration module 2006 may employ techniques such as centrifugation, size exclusion filtration, or affinity-based separation to isolate the relevant fraction of the blood sample containing the cancer biomarkers of interest. The module may incorporate multi-stage filtration processes, combining different techniques to achieve optimal sample purity. Additionally, the filtration module may utilize microfluidic devices with integrated membrane filters to enable efficient and automated sample processing at small volumes.

The filtration process performed by the sample filtration module 2006 may be tailored to the specific cancer biomarkers being targeted by the cancer detection system 2000. For example, different filtration parameters may be used when preparing samples for detection of circulating tumor cells versus soluble protein biomarkers. The module may include programmable settings that automatically adjust filtration protocols based on the selected biomarker panel. Furthermore, the filtration process may incorporate quality control checkpoints, such as spectrophotometric analysis, to ensure that the filtered samples meet predefined purity and concentration standards before proceeding to the bioFET array.

The filtered samples from the sample preparation unit 2002 are analyzed using a bioFET array 2008, which may contain multiple bioFET sensors including an HE4 bioFET 2010, a CA 125 bioFET 2012, and a VEGF bioFET 2014. It is recognized that the bioFET sensors included in the bioFET array 2008 are merely exemplary, and other configurations of bioFET arrays may include other bioFETs configured to detect specific biomarkers.

For example, each bioFET in the array 2008 may be configured to detect specific cancer biomarkers associated with different types or stages of cancer. The bioFET array 2008 may be fabricated using advanced microfabrication techniques to ensure uniformity and reproducibility across all sensors. Additionally, the array may incorporate on-chip reference electrodes and temperature sensors to enable real-time calibration and environmental compensation during measurements.

As a specific example, the HE4 bioFET 2010 may be functionalized with nanobodies or aptamers specific to the Human Epididymis Protein 4 (HE4) biomarker, which is associated with ovarian cancer. The CA 125 bioFET 2012 may be designed to detect the Cancer Antigen 125 (CA 125) protein, another important marker for ovarian cancer. The VEGF bioFET 2014 may be functionalized to detect Vascular Endothelial Growth Factor (VEGF), a biomarker associated with angiogenesis in various cancer types. Each bioFET may utilize a unique surface chemistry optimized for its specific target biomarker, potentially incorporating nanomaterials or polymer brushes to enhance sensitivity and reduce non-specific binding. The functionalization process may also include the use of oriented immobilization techniques to maximize the binding capacity and accessibility of the recognition elements.

The signals from the bioFET array 2008 are processed by a signal processing unit 2016, which feeds the processed data to a data analysis module 2018. In various embodiments, the signal processing unit 2016 may amplify and filter the electrical signals from each bioFET in the array 2008, converting the raw sensor outputs into a format suitable for digital analysis. The signal processing unit 2016 may employ high-precision, low-noise amplifiers with programmable gain settings to accommodate the wide dynamic range of biomarker concentrations. Additionally, the unit may incorporate real-time signal quality assessment algorithms to identify and flag potentially erroneous measurements for further investigation.

The signal processing unit 2016 may employ other signal processing techniques such as noise reduction algorithms, drift correction, and/or differential measurements to enhance the sensitivity and specificity of biomarker detection. These techniques may enable the cancer detection system 2000 to detect biomarkers at ultra-low concentrations (e.g. as low as 1-10 femtomolar, etc.), potentially allowing for earlier cancer detection. The unit may utilize adaptive filtering techniques that continuously optimize filter parameters based on the characteristics of the incoming signals. Furthermore, the signal processing unit 2016 may implement parallel processing architectures to handle the high data throughput from multiple bioFET sensors simultaneously, reducing overall analysis time.

Additionally, the data analysis module 2018 may analyze the processed signals to determine the presence and levels of cancer biomarkers. The data analysis module 2018 may utilize machine learning algorithms trained on large datasets of cancer biomarker profiles to interpret the complex patterns of biomarker expression detected by the bioFET array 2008. The module may employ ensemble learning techniques, combining multiple machine learning models to improve prediction accuracy and robustness. Additionally, the data analysis module 2018 may incorporate transfer learning capabilities, allowing it to adapt pre-trained models to new biomarker combinations or patient populations with minimal retraining.

The data analysis performed by the data analysis module 2018 may consider the relative levels of multiple biomarkers and their known associations with various cancer types. For example, the combined analysis of HE4 and CA 125 levels may provide a more accurate assessment of ovarian cancer risk than either biomarker alone. The module may utilize other statistical techniques, such as multivariate analysis and Bayesian inference, to integrate data from multiple biomarkers and calculate comprehensive risk scores. Furthermore, the data analysis module 2018 may incorporate longitudinal analysis capabilities, tracking changes in biomarker levels over time to identify trends that may indicate disease progression or treatment response.

The results from the data analysis module 2018 are presented through a user interface 2020, which displays the detection results. In operation 2020, the user interface 2020 may provide a clear and intuitive presentation of the cancer biomarker analysis results, potentially including visualizations of biomarker levels, risk assessments, and recommendations for further diagnostic procedures. The user interface may utilize interactive data visualization techniques, such as dynamic charts and heatmaps, to allow users to explore the relationships between different biomarkers and clinical outcomes. Additionally, the interface may incorporate customizable reporting templates that can be tailored to the specific needs of different healthcare institutions or clinical workflows.

The user interface 2020 may be designed to cater to both healthcare professionals and patients, offering different levels of detail and explanation based on the user's needs. For healthcare professionals, the interface may provide in-depth data on biomarker concentrations and statistical analyses, while for patients, it may offer simplified risk assessments and next-step recommendations. The interface may include role-based access controls and customizable dashboards to ensure that each user group receives appropriate information. Furthermore, the user interface 2020 may incorporate natural language processing capabilities to generate plain-language summaries of complex biomarker data, making the results more accessible to patients and non-specialist healthcare providers.

The cancer detection system 2000 demonstrates an advancement to challenges in traditional cancer screening methods, which often rely on single biomarker tests or invasive procedures. By integrating sample preparation, multi-biomarker detection using bioFETs, and advanced data analysis in a single platform, the cancer detection system 2000 offers a comprehensive and potentially more sensitive approach to cancer screening. Furthermore, and as has been discussed throughout this disclosure, the cancer detection system 2000 addresses limitations in conventional cancer biomarker detection by leveraging the unique properties of bioFETs and the specificity of nanobody or aptamer functionalization. This combination may result in improved sensitivity and selectivity compared to traditional immunoassays, potentially enabling the detection of cancer biomarkers at earlier stages of disease progression.

In various embodiments, the cancer detection system 2000 may be expanded to include additional bioFETs functionalized for other cancer biomarkers, allowing for the detection of a wider range of cancer types. For example, bioFETs specific to prostate-specific antigen (PSA) or circulating tumor DNA (ctDNA) may be added to enhance the system's capabilities for detecting prostate cancer or monitoring treatment response.

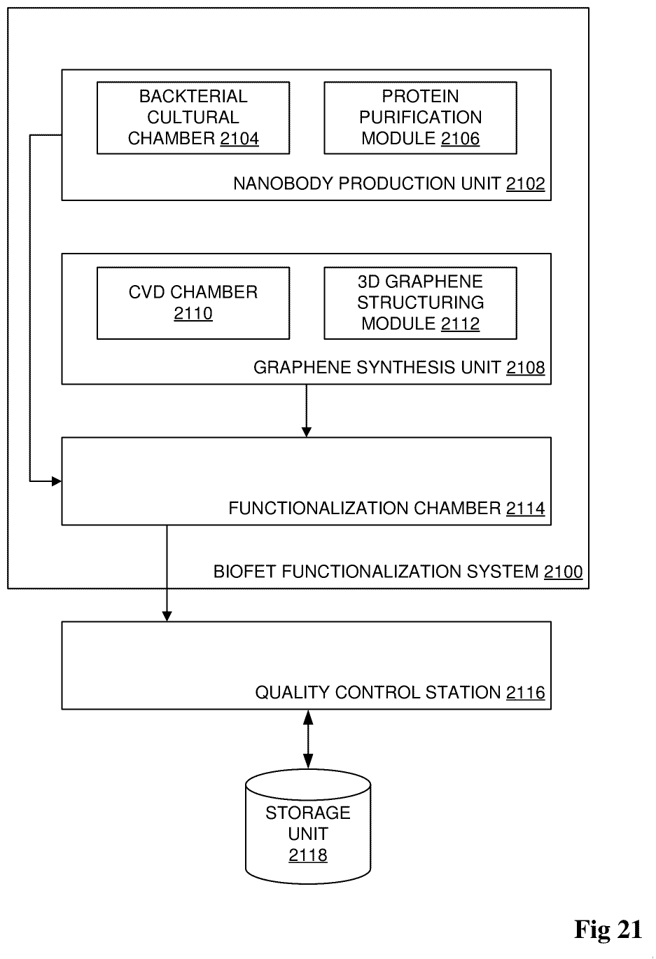

FIG. 21 illustrates a block diagram of a bioFET functionalization system 2100, in accordance with one embodiment. As an option, the bioFET functionalization system 2100 may be implemented in the context of any one or more of the embodiments set forth in any previous and/or subsequent Figures and/or description thereof. Of course, however, the bioFET functionalization system 2100 may be implemented in the context of any desired environment. Further, the aforementioned definitions may equally apply to the description below.

The bioFET functionalization system 2100 includes a nanobody production unit 2102, a graphene synthesis unit 2108, and a functionalization chamber 2114. In operation, these components work together to produce functionalized bioFETs for cancer biomarker detection, with potential applications in detecting biomarkers for other diseases as well. The system may be designed with modular components that can be easily upgraded or replaced to accommodate new nanobody production techniques or graphene synthesis methods. Additionally, the system may incorporate real-time monitoring and feedback mechanisms to optimize the production process and ensure consistent quality of the functionalized bioFET s.

The nanobody production unit 2102 contains a bacterial cultural chamber 2104 and a protein purification module 2106. In operation, the bacterial cultural chamber 2104 may be used to cultivate bacteria engineered to express specific nanobodies targeting cancer biomarkers. The chamber may be equipped with temperature and pH control systems to optimize nanobody production. The bacterial cultural chamber 2104 may also include advanced bioreactor technologies, such as perfusion systems or microcarrier cultures, to enhance nanobody yield and quality. Furthermore, the chamber may be designed with built-in contamination detection and prevention measures to ensure the purity of the bacterial cultures.

The bacterial cultural chamber 2104 may be designed to accommodate different bacterial strains for producing a variety of nanobodies. This versatility may allow the bioFET functionalization system 2100 to create bioFETs for detecting multiple cancer types or other diseases by simply changing the bacterial culture. The chamber may incorporate automated strain switching mechanisms to facilitate rapid transitions between different nanobody production runs. Additionally, it may feature a library of pre-optimized growth protocols for various bacterial strains, enabling quick adaptation to different nanobody production requirements.

Further, the protein purification module 2106 may extract and purify the nanobodies produced in the bacterial cultural chamber 2104. This module may employ techniques such as affinity chromatography or size exclusion chromatography to isolate the desired nanobodies from other cellular components. The purification process may be further enhanced by incorporating multi-step purification strategies, such as combining ion exchange chromatography with hydrophobic interaction chromatography, to achieve higher purity and yield. The module may also utilize advanced membrane technologies, such as tangential flow filtration, to concentrate and buffer-exchange the nanobodies efficiently.

The protein purification module 2106 may be equipped with automated systems for handling multiple purification steps, ensuring consistent quality of the purified nanobodies. In some cases, the module may also perform quality control tests to verify the purity and functionality of the nanobodies before they are used in bioFET functionalization. The automated systems may include robotic sample handling and intelligent process control algorithms that can adapt purification parameters in real-time based on sensor feedback. Additionally, the quality control tests may incorporate high-throughput analytical techniques, such as mass spectrometry or surface plasmon resonance, to provide comprehensive characterization of the purified nanobodies.

The graphene synthesis unit 2108 includes a CVD chamber 2110 and a graphene structuring module 2112. In operation, the CVD chamber 2110 may produce graphene material using chemical vapor deposition techniques. This process may involve the decomposition of carbon-containing gases on a metal substrate under controlled temperature and pressure conditions. The CVD chamber may be equipped with advanced gas delivery systems that allow precise control over the composition and flow rates of precursor gases, enabling fine-tuning of graphene properties. Furthermore, the chamber may incorporate in-situ monitoring tools, such as Raman spectroscopy or ellipsometry, to provide real-time feedback on graphene growth and quality.

The CVD chamber 2110 may be designed to produce high-quality, large-area graphene sheets with minimal defects. In some cases, the chamber may be capable of producing both single-layer and multi-layer graphene, allowing for flexibility in bioFET design based on specific sensing requirements. The chamber may utilize advanced substrate preparation techniques, such as electropolishing or annealing, to improve the uniformity and quality of the graphene growth. Additionally, it may feature a roll-to-roll production capability for continuous graphene synthesis, enabling high-throughput production of graphene sheets for large-scale bioFET manufacturing.

Additionally, the graphene structuring module 2112 may process the graphene produced by the CVD chamber 2110 to create three-dimensional graphene structures. This module may use techniques such as etching, folding, or controlled growth to transform the flat graphene sheets into 3D structures with increased surface area. The module may incorporate advanced nanofabrication tools, such as focused ion beam milling or electron beam lithography, to create precise and reproducible 3D graphene architectures. Furthermore, it may utilize self-assembly techniques or template-assisted growth methods to generate complex 3D graphene structures with tailored porosity and surface properties.

The graphene structuring module 2112 may employ advanced patterning techniques to create specific 3D graphene morphologies optimized for different types of cancer biomarkers. For example, certain structures may be more suitable for detecting protein biomarkers, while others may be optimized for nucleic acid detection. The module may utilize computational modeling and machine learning algorithms to predict and design optimal 3D graphene structures for specific biomarker interactions. Additionally, it may feature a library of pre-designed 3D graphene templates that can be rapidly customized and fabricated for different biomarker detection applications.

The functionalization chamber 2114 receives materials from both the nanobody production unit 2102 and graphene synthesis unit 2108 to create functionalized bioFETs. The chamber may use techniques such as covalent bonding or physical adsorption to attach the purified nanobodies to the 3D graphene structures. The chamber may incorporate advanced surface modification techniques, such as plasma treatment or click chemistry, to enhance the efficiency and stability of nanobody attachment. Furthermore, it may utilize controlled atmosphere systems to prevent contamination and ensure optimal reaction conditions during the functionalization process.

The functionalization chamber 2114 may be equipped with precise environmental controls to ensure optimal conditions for nanobody attachment. In some cases, the chamber may also incorporate microfluidic systems for efficient and uniform distribution of nanobodies across the graphene surface. The environmental control systems may include advanced humidity regulation and gas composition management to maintain ideal conditions for different functionalization chemistries. Additionally, the microfluidic systems may feature programmable flow patterns and gradient generators to create spatially controlled functionalization of the graphene surfaces, enabling the production of multi-analyte bioFET arrays.

The functionalized bioFETs then pass through a quality control station 2116 for verification and testing. For example, the quality control station 2116 may perform a series of electrical and biochemical tests to ensure the functionality and sensitivity of the bioFETs. These tests may include measurements of electrical characteristics and binding affinity for target cancer biomarkers. The station may incorporate high-throughput impedance spectroscopy systems to rapidly assess the electrical properties of multiple bioFETs simultaneously. Furthermore, it may utilize automated microfluidic handling systems to perform parallel binding assays with multiple cancer biomarkers, providing comprehensive functional characterization of the bioFETs.

The quality control station 2116 may use automated testing systems to assess multiple bioFETs simultaneously, improving throughput and consistency. In some cases, the station may also perform accelerated stability tests to predict the long-term performance of the functionalized bioFETs under various storage and usage conditions. The automated testing systems may incorporate machine vision and artificial intelligence algorithms to detect and classify defects or performance issues in the bioFETs. Additionally, the accelerated stability tests may utilize environmental simulation chambers that can rapidly cycle through various temperature, humidity, and chemical exposure conditions to evaluate bioFET durability and reliability.

A storage unit 2118 connects to the quality control station 2116 to store the verified functionalized bioFETs. For example, the storage unit 2118 may maintain appropriate conditions such as temperature, humidity, and protection from light to preserve the bioFETs until they are needed for use in cancer detection applications. The storage unit may feature advanced climate control systems with redundant backup power to ensure uninterrupted maintenance of optimal storage conditions. Furthermore, it may incorporate inert gas purging systems to prevent oxidation or degradation of sensitive bioFET components during long-term storage.

Still yet, the storage unit 2118 may be equipped with inventory management systems to track the production date, target biomarker, and quality control data for each batch of bioFETs. This information may be used to ensure proper rotation of stock and to provide traceability in case of any quality issues. The inventory management system may utilize RFID tagging or barcode scanning technologies to automate tracking and reduce human error in inventory management. Additionally, it may feature predictive analytics capabilities to optimize stock levels and production schedules based on historical usage patterns and projected demand for different types of bioFETs.

The bioFET functionalization system 2100 exemplifies a resolution to challenges in traditional biosensor production methods, which often involve complex, multi-step processes with potential for variability and contamination. By integrating nanobody production, graphene synthesis, and functionalization in a single, controlled system, the bioFET functionalization system 2100 offers a streamlined and potentially more reliable approach to producing high-quality bioFETs for cancer detection.

Furthermore, the bioFET functionalization system 2100 addresses limitations in conventional biosensor manufacturing by leveraging the unique properties of 3D graphene structures and the specificity of nanobodies. This combination may result in bioFETs with improved sensitivity and selectivity compared to traditional flat substrate sensors, potentially enabling the detection of cancer biomarkers at earlier stages or lower concentrations.

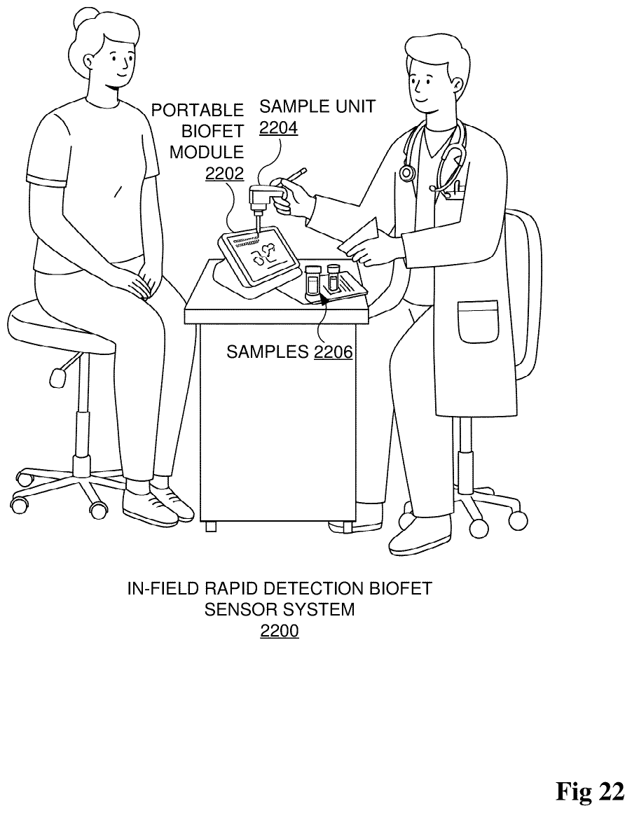

FIG. 22 illustrates an orthogonal view of a biosensor system 2200 for biomarker detection, in accordance with one embodiment. As an option, the biosensor system 2200 may be implemented in the context of any one or more of the embodiments set forth in any previous and/or subsequent Figures and/or description thereof. Of course, however, the biosensor system 2200 may be implemented in the context of any desired environment. Further, the aforementioned definitions may equally apply to the description below.

The biosensor system 2200 includes a portable bioFET module 2202 and a sample unit 2204. In operation, the portable bioFET module 2202 may be positioned adjacent to the sample unit 2204, which contains samples 2206. The sample unit 2204 may be configured to hold and organize the samples 2206 for testing using the portable bioFET module 2202. The modular design of the system allows for easy replacement or upgrade of individual components, enhancing the system's versatility and longevity. The interface between the portable bioFET module 2202 and sample unit 2204 may incorporate precision alignment mechanisms to ensure accurate and reproducible positioning for each test.

The portable bioFET module 2202 may have a compact design that enables portability while maintaining functionality for biomarker detection, as has been discussed herein. In some cases, the portable bioFET module 2202 may incorporate miniaturized versions of components similar to those found in the cancer detection system 2000, such as a bioFET array and signal processing capabilities, as well as the units shown in FIGS. 13 and 14. Further, the miniaturization process may involve advanced packaging techniques, such as 3D chip stacking or system-on-chip integration, to reduce the overall size and power consumption of the module. Additionally, the portable bioFET module 2202 may utilize flexible or stretchable electronics to enhance durability and conform to various testing environments.

The biosensor system 2200 may be designed for rapid, on-site detection of cancer biomarkers in various settings, such as clinics, mobile health units, or even patients' homes. The portability of the system may allow for early screening and monitoring of cancer biomarkers in resource-limited environments or for frequent testing during cancer treatment. The system may be equipped with ruggedized components and protective casings to withstand diverse environmental conditions, ensuring reliable operation in field settings. Additionally, the biosensor system 2200 may incorporate wireless connectivity features to enable remote data transmission and real-time consultation with healthcare professionals.

In operation, the portable bioFET module 2202 may interface with the sample unit 2204 to perform automated analysis of the samples 2206. This may involve the transfer of one or more small volumes of sample from the sample unit 2204 to the bioFET sensors within the portable bioFET module 2202, followed by electrical measurements to detect the presence of specific cancer biomarkers. The sample transfer process may utilize precision microfluidics with integrated bubble detection and removal mechanisms to ensure accurate and contamination-free sample delivery. The system may also incorporate adaptive measurement protocols that can adjust sensing parameters in real-time based on initial sample characteristics, optimizing detection sensitivity for each individual test.

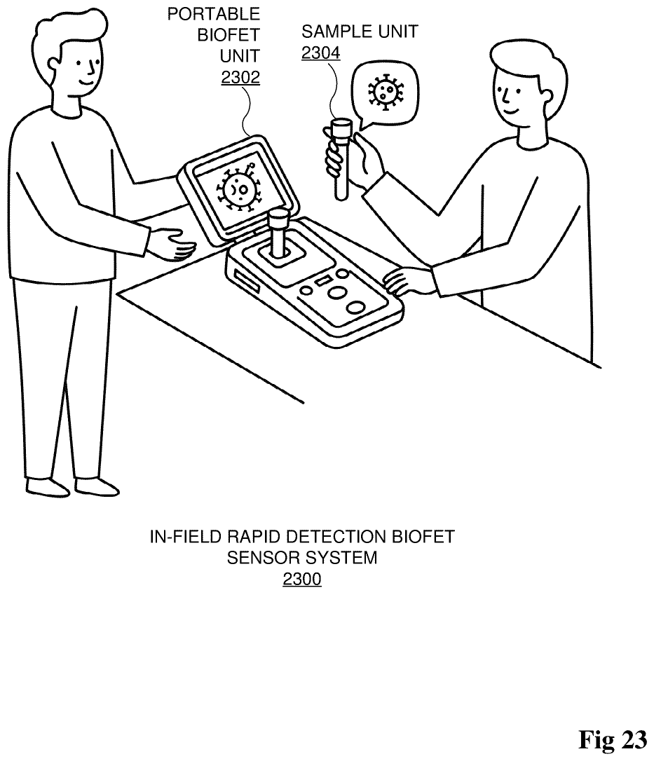

FIG. 23 illustrates a schematic view of an in-field rapid detection bioFET sensor system 2300, in accordance with one embodiment. As an option, the in-field rapid detection bioFET sensor system 2300 may be implemented in the context of any one or more of the embodiments set forth in any previous and/or subsequent Figures and/or description thereof. Of course, however, the in-field rapid detection bioFET sensor system 2300 may be implemented in the context of any desired environment. Further, the aforementioned definitions may equally apply to the description below.

The system includes a portable bioFET module 2302 and a sample unit 2304. In operation, the portable bioFET module 2302 may contain sensing elements for detecting biomarkers, while the sample unit 2304 may be configured to hold test samples for analysis. The portable bioFET module 2302 may incorporate shock-absorbing materials and stabilizing mechanisms to ensure reliable operation even in challenging field conditions. The sample unit 2304 may feature a locking mechanism that securely attaches it to the portable bioFET module 2302, maintaining proper alignment during the testing process.

The schematic view in FIG. 23 may illustrate how the system 2300 can be used for rapid in-field detection of various biomarkers. In some cases, the portable nature of the system may allow for its use in non-traditional healthcare settings, potentially increasing access to cancer screening and monitoring services. The system's compact design may enable its integration into mobile health units or temporary screening facilities set up in remote areas. Additionally, the portability of the system 2300 may facilitate its use in emergency response scenarios, allowing for rapid biomarker screening in disaster-affected regions.

The portable bioFET module 2302 may incorporate an automated fluidics-based test setup, similar to the one described in relation to the cancer detection system 2000, as well as the units shown in FIGS. 13 and 14. This automated system may handle sample preparation, delivery to the bioFET sensors, and subsequent washing steps, minimizing the need for manual intervention and reducing the potential for human error. The fluidics system may utilize microfluidic channels with specialized coatings to prevent sample adhesion and cross-contamination between tests. Furthermore, the system may incorporate on-board quality control measures, such as integrated calibration standards, to ensure the reliability of each test run.

In operation, the sample unit 2304 may interface with the portable bioFET module 2302 through a standardized connection, allowing for secure and precise transfer of samples. The automated fluidics system within the portable bioFET module 2302 may then control the flow of samples and reagents across the bioFET sensors, enabling precise and reproducible measurements. The interface may include self-aligning connectors and fail-safe mechanisms to prevent incorrect sample loading or leakage. Additionally, the system may incorporate real-time flow monitoring to detect and correct any irregularities in sample or reagent delivery during the testing process.

The biosensor system 2300 exemplifies a resolution to challenges in traditional cancer biomarker detection methods, which often require large, stationary laboratory equipment and specialized personnel. By providing a portable, automated system for biomarker detection, the biosensor system 2300 may enable more widespread and frequent cancer screening, potentially leading to earlier detection and improved patient outcomes. The system's user-friendly interface may allow operation by healthcare workers with minimal specialized training, expanding its potential deployment in underserved areas. Moreover, the rapid turnaround time for results may reduce patient anxiety and enable faster clinical decision-making.

Furthermore, the biosensor system 2300 addresses limitations in conventional point-of-care diagnostics by leveraging the high sensitivity and specificity of bioFET technology. The combination of 3D graphene-based bioFETs and automated fluidics may allow for the detection of cancer biomarkers at lower concentrations than traditional rapid tests, potentially rivaling the performance of laboratory-based assays. The system may incorporate advanced signal amplification techniques, such as enzymatic signal enhancement or nanoparticle-based detection, to further improve sensitivity. Additionally, the use of multiplexed bioFET arrays may enable simultaneous detection of multiple biomarkers, enhancing the overall diagnostic accuracy.

In various embodiments, the biosensor system 2300 may be expanded to include additional sensing modalities beyond bioFETs. For example, the system may incorporate optical or electrochemical sensors to provide complementary data on cancer biomarkers, enhancing the overall diagnostic accuracy of the device. The integration of multiple sensing modalities may allow for cross-validation of results, reducing the likelihood of false positives or negatives. Furthermore, the system may include capabilities for measuring physical parameters such as sample viscosity or conductivity, providing additional contextual information for more accurate biomarker analysis.

In various embodiments, the sample unit 2304 may be designed as a disposable cartridge pre-loaded with necessary reagents and calibration standards. This approach may simplify the testing process and ensure consistency across different testing environments, from clinical settings to remote field locations. The cartridge design may incorporate features such as lyophilized reagents for extended shelf life and built-in quality indicators to verify reagent integrity before use. Additionally, the cartridge may include multiple chambers for parallel processing of different biomarkers or for running control samples alongside patient samples.

In various embodiments, the portable bioFET module 2302 may be equipped with wireless communication capabilities, allowing for real-time data transmission to healthcare providers or centralized databases. This connectivity may enable remote monitoring of cancer biomarkers, facilitate telemedicine consultations, and contribute to large-scale epidemiological studies on cancer prevalence and progression. The wireless system may utilize encryption protocols to ensure patient data security and comply with healthcare privacy regulations. Furthermore, the module may include a local data storage capability to allow operation in areas with limited connectivity, with automatic data synchronization when a connection becomes available.

### Use Case Scenario 1: Rapid COVID-19 Detection in Remote Areas

A mobile health unit equipped with the portable bioFET system arrives in a rural village with limited access to healthcare facilities. The team sets up a temporary testing station to screen the local population for COVID-19. The bioFET array is functionalized with nanobodies specific to SARS-COV-2 spike proteins. Villagers provide saliva samples, which are quickly processed using the integrated sample preparation unit. The automated system applies the samples to the bioFET array, and within 15 minutes, results are available. The portable system can process up to 50 samples per hour, allowing for rapid community-wide screening. Positive cases are immediately identified and isolated, preventing further spread. The cost per test is estimated at $5, compared to $50-100 for traditional PCR tests. The speed and affordability of the bioFET system enable frequent testing and monitoring of the village population, effectively controlling the outbreak without the need for expensive laboratory infrastructure or skilled technicians.

### Use Case Scenario 2: Early-Stage Pancreatic Cancer Screening Program

A hospital implements a pancreatic cancer screening program using the portable bioFET system for high-risk individuals. The bioFET array is functionalized with nanobodies targeting multiple early-stage pancreatic cancer biomarkers, including CA 19-9, TIMP-1, and LRG1. Patients provide a small blood sample, which is processed on-site. The bioFET system analyzes the sample for the presence of these biomarkers at femtomolar concentrations, a level of sensitivity not achievable with traditional ELISA tests. Results are available within 30 minutes, allowing for immediate consultation with an oncologist if biomarkers are detected. The early detection capabilities of the bioFET system enable the identification of pancreatic cancer at Stage I or II, significantly improving treatment outcomes. The cost per test is approximately $50, compared to $500-1000 for a combination of traditional biomarker tests and imaging studies. This affordability allows for more frequent screening of high-risk individuals, potentially saving lives through early detection.

### Use Case Scenario 3: Environmental Toxin Monitoring in Water Sources

An environmental protection agency deploys portable bioFET systems to monitor water sources for the presence of harmful toxins and pollutants. The bioFET arrays are functionalized with recognition elements for various contaminants, including heavy metals, pesticides, and industrial chemicals. Field technicians collect water samples from rivers, lakes, and groundwater sources. The samples are analyzed on-site using the bioFET system, with results available in under an hour. The system can detect contaminants at parts-per-billion levels, rivaling the sensitivity of laboratory-based mass spectrometry techniques. The rapid, on-site analysis allows for immediate action if contamination is detected, such as issuing public warnings or tracing pollution sources. The cost per analysis is estimated at $20, compared to $200-500 for traditional laboratory testing. This cost-effectiveness enables more frequent and widespread monitoring of water sources, significantly improving public health protection and environmental conservation efforts.

### Use Case Scenario 4: Rapid Food Safety Testing in the Supply Chain

A large food distribution company implements portable bioFET systems for rapid testing of food products throughout the supply chain. The bioFET arrays are functionalized with antibodies and aptamers targeting common foodborne pathogens such as E. coli, Salmonella, and Listeria. Quality control personnel at distribution centers use the bioFET system to test samples from incoming shipments. The automated sample preparation unit can handle various food matrices, from produce to meat products. Results are available within 2 hours, compared to 24-48 hours for traditional culture-based methods. The speed of detection allows for real-time decision-making on whether to accept or reject shipments, reducing the risk of contaminated products entering the market. The cost per test is approximately $10, compared to $50-100 for traditional pathogen testing methods. This affordability enables more frequent testing at multiple points in the supply chain, significantly enhancing food safety measures and reducing the incidence of foodborne illnesses.

### Use Case Scenario 5: Early Detection of Ovarian Cancer

A network of primary care clinics implements a screening program for ovarian cancer using the portable bioFET system. The bioFET array is functionalized with nanobodies specific to multiple ovarian cancer biomarkers, including HE4, CA 125, and VEGF, enabling a multi-analyte detection approach. Women visiting their primary care physician for routine check-ups provide a small blood sample, which is immediately processed using the integrated sample preparation unit. The automated system applies the sample to the bioFET array, and within 20 minutes, results for all biomarkers are available. The portable bioFET system can detect these biomarkers at femtomolar concentrations, allowing for the identification of ovarian cancer at very early stages when traditional methods might miss it. The multi-biomarker approach significantly reduces false positives and negatives compared to single-marker tests. Results are instantly analyzed by the system's built-in algorithms, which consider the combination and levels of biomarkers to assess cancer risk. If elevated risk is detected, the patient can be immediately referred for further diagnostic procedures such as transvaginal ultrasound or consultation with a gynecologic oncologist. The cost per test is approximately $30, compared to $200-400 for traditional biomarker assays. This affordability allows for more frequent screening, especially for women with high-risk factors. The speed and accessibility of testing in primary care settings encourage more women to undergo regular screening, potentially leading to earlier detection and improved survival rates for ovarian cancer.

Taking a step back, in all these scenarios, the portable bioFET system demonstrates its ability to provide rapid, sensitive, and cost-effective testing in various fields. Its portability and ease of use allow for on-site analysis, eliminating the need for sample transportation and reducing turnaround times. The significant cost savings per test, coupled with increased testing frequency, represent a paradigm shift in diagnostic and monitoring capabilities across multiple industries.

Additionally, the portable nature of the system allows for its use in mobile health units, bringing this advanced screening capability to underserved or rural areas where access to specialized oncology services may be limited. This democratization of advanced diagnostic technology could significantly impact ovarian cancer outcomes by enabling widespread, early-stage detection.

The present disclosure addresses significant challenges in the field of portable biosensing that have limited the effectiveness of existing technologies. Prior art solutions have struggled to achieve the sensitivity and specificity of laboratory-based methods while maintaining a compact form factor suitable for field deployment. Conventional portable biosensors often lack sophisticated control over sample exposure and measurement conditions, leading to inconsistent results. Additionally, many current systems face issues related to sensor stability, cross-reactivity with interfering substances, and the ability to detect multiple analytes simultaneously. These limitations have hindered the widespread adoption of portable biosensors in critical applications such as environmental monitoring, healthcare diagnostics, and food safety testing.

The disclosed automated portable biosensor system overcomes these deficiencies through a novel approach that uses three-dimensional graphene-based sensing elements. By utilizing vertical graphene field effect transistor (FET) arrays, the system achieves high sensitivity and specificity in a compact, field-deployable format. The vertical graphene structure provides increased surface area for analyte binding, while the automated sample handling and measurement processes ensure consistent and reliable results without the need for specialized operators. This innovative approach not only enables rapid, on-site analysis of liquid samples but also offers the flexibility to detect a wide range of analytes through its modular design, effectively addressing the longstanding issues of portability, sensitivity, and versatility that have plagued prior art biosensing systems.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A biosensor comprising:
a substrate;
an array of three-dimensional vertical graphene structures disposed on the substrate;
electrodes in electrical contact with the vertical graphene structures; and
a functionalization layer on surfaces of the vertical graphene structures, wherein the functionalization layer comprises bioreceptors configured to bind to specific target analytes.

2. The biosensor of claim 1, wherein the vertical graphene structures have a height ranging from 300 nm to 500 nm.

3. The biosensor of claim 2, wherein the vertical graphene structures exhibit a jagged surface morphology.

4. The biosensor of claim 1, wherein the substrate comprises silicon dioxide.

5. The biosensor of claim 1, wherein the electrodes comprise a source electrode and a drain electrode positioned on opposite sides of each vertical graphene structure.

6. The biosensor of claim 5, further comprising a gate electrode positioned adjacent to the vertical graphene structures.

7. The biosensor of claim 6, wherein:
the gate electrode is configured to apply a voltage ranging from −0.1V to 0.9V; and
a bias voltage of 50-300 mV is applied to the source and drain electrodes during measurement.

8. The biosensor of claim 1, wherein the bioreceptors comprise at least one of antibodies, nucleic acids, or proteins.

9. The biosensor of claim 1, further comprising a microfluidic system configured to deliver liquid samples to the functionalized vertical graphene structures.

10. The biosensor of claim 9, wherein the microfluidic system comprises:
at least one sample well for containing a liquid sample;
a microfluidic channel connecting the sample well to the functionalized vertical graphene structures; and
a pump for controlling fluid flow through the microfluidic channel.

11. The biosensor of claim 10, further comprising a flow sensor for monitoring fluid flow rates through the microfluidic system.

12. The biosensor of claim 1, further comprising a measurement circuit configured to detect changes in electrical properties of the vertical graphene structures upon binding of target analytes to the bioreceptors.

13. The biosensor of claim 12, wherein the measurement circuit is configured to perform current drift correction on sensor responses.

14. The biosensor of claim 12, wherein the measurement circuit is configured to sweep through a range of applied voltages when measuring sensor responses.

15. The biosensor of claim 1, further comprising a controller configured to:
control exposure of liquid samples to the functionalized vertical graphene structures;
measure sensor responses from the vertical graphene structures; and
process data from the sensor responses to determine analyte concentrations in the liquid samples.

16. The biosensor of claim 15, wherein the controller is further configured to certify minimal sensor performance before proceeding with sample exposure.

17. The biosensor of claim 1, wherein the functionalization layer comprises amine diazonium groups covalently bonded to the surfaces of the vertical graphene structures.

18. The biosensor of claim 17, wherein the bioreceptors are attached to the amine diazonium groups via EDC/NHS (1-Ethyl-3-(3-dimethylaminopropyl) carbodiimide/N-hydroxysuccinimide) chemistry.

19. The biosensor of claim 1, further comprising:
a housing enclosing the substrate, vertical graphene structures, and electrodes;
a display screen on an exterior surface of the housing for providing visual information to a user; and
at least one microfluidic port on an exterior surface of the housing for introducing liquid samples.

20. The biosensor of claim 19, further comprising a wireless communication module configured to transmit measurement data to a remote device.

21. The biosensor of claim 1, wherein:
the vertical graphene structures have a height ranging from 300 nm to 500 nm;
the vertical graphene structures exhibit a jagged surface morphology; and
the vertical graphene structures are configured to extend into the liquid sample beyond the Debye screening length, enabling more efficient interaction with target analytes.

22. The biosensor of claim 1, wherein:
the vertical graphene structures are grown in-situ directly on the substrate;
the in-situ growth process results in improved adhesion and electrical contact between the vertical graphene structures and the electrodes compared to monolayer graphene; and
the vertical graphene structures have a non-uniform surface morphology with varying heights to increase surface area for analyte binding.

23. The biosensor of claim 1, further comprising:
a microfluidic system configured to deliver liquid samples to the functionalized vertical graphene structures;
a measurement circuit configured to detect changes in electrical properties of the vertical graphene structures upon binding of target analytes to the bioreceptors; and
a controller configured to process data from the sensor responses to determine analyte concentrations in the liquid samples.

24. The biosensor of claim 1, wherein:
the biosensor is configured as a field-deployable device;
the field-deployable device includes a display for real-time data visualization, a PCB assembly for electronic control and signal processing, and microfluidic ports for sample introduction and management; and
the field-deployable device is configured for on-site sample analysis without the need for complex laboratory equipment.

25. The biosensor of claim 1, wherein the specific target analytes are cancer biomarkers.

26. The biosensor of claim 25, wherein at least one:
the cancer biomarkers are associated with pancreatic cancer; or
the cancer biomarkers are associated with ovarian cancer.

27. The biosensor of claim 25, wherein at least one of:
the biosensor is capable of detecting the cancer biomarkers at concentrations as low as 1-10 femtomolar;
the cancer biomarkers include at least one of CA 19-9, TIMP-1, or LRG1; or
the cancer biomarkers include at least one of HE4, CA 125, or VEGF.

28. The biosensor of claim 25, wherein:
the array of vertical graphene structures comprises multiple sensing elements arranged in a planar or stacked configuration; and
each sensing element is functionalized to detect a different cancer biomarker or group of cancer biomarkers.

29. The biosensor of claim 25, wherein the vertical graphene structures form a permittivity gradient.

30. The biosensor of claim 29, wherein the permittivity gradient corresponds to at least one of a stair shaped gradient, a linearly shaped gradient, or a curvilinearly shaped gradient.

31. The biosensor of claim 25, wherein:
the bioreceptors include nanobodies specific to the cancer biomarkers; and
the nanobodies are engineered to include specific tags or linker molecules to facilitate their attachment to the graphene surface while maintaining optimal orientation for cancer biomarker binding.

32. The biosensor of claim 25, wherein the biosensor is configured to simultaneously detect multiple cancer biomarkers from a single patient sample.

33. The biosensor of claim 25, wherein the biosensor is configured to detect early-stage cancer biomarkers before the onset of clinical symptoms.

34. The biosensor of claim 1, wherein:
the specific target analytes include viral antigens; and
the biosensor is configured to detect viral infections, including SARS-COV-2.

35. The biosensor of claim 34, wherein:
the bioreceptors include nanobodies specific to SARS-COV-2 spike proteins; and
the biosensor is capable of detecting SARS-COV-2 antigens in saliva samples.

36. The biosensor of claim 1, wherein:
the specific target analytes include environmental toxins; and
the biosensor is configured to detect contaminants in water sources.

37. The biosensor of claim 36, wherein:
the environmental toxins include heavy metals, pesticides, and industrial chemicals; and
the biosensor is capable of detecting contaminants at parts-per-billion levels.

38. The biosensor of claim 1, wherein:
the specific target analytes include foodborne pathogens; and
the biosensor is configured to detect common foodborne pathogens in food products.

39. The biosensor of claim 38, wherein:
the foodborne pathogens include E. coli, Salmonella, and Listeria; and
the biosensor is capable of providing results within 2 hours of sample application.

40. The biosensor of claim 1, wherein:
the biosensor is configured to detect multiple classes of analytes, including cancer biomarkers, viral antigens, environmental toxins, and foodborne pathogens;
the biosensor includes multiple functionalized sensing elements, each optimized for a specific class of analytes; and
the biosensor is capable of providing rapid, on-site detection.
